SEQUENCE LISTING (145 sequences)

SEQ ID NO: 1 - protein, 8 aa, synthetic construct
  REGION 1..8: note = Description of sequence: Antibody component
GYTFTAYY

SEQ ID NO: 2 - protein, 8 aa, synthetic construct
  REGION 1..8: note = Description of sequence: Antibody component
INPNSGGT

SEQ ID NO: 3 - protein, 18 aa, synthetic construct
  REGION 1..18: note = Description of sequence: Antibody component
TRGTPRYNWKSYNYGMDV

SEQ ID NO: 4 - protein, 11 aa, synthetic construct
  REGION 1..11: note = Description of sequence: Antibody component
QSLVHSDGNTY

SEQ ID NO: 5 - skipped

SEQ ID NO: 6 - protein, 9 aa, synthetic construct
  REGION 1..9: note = Description of sequence: Antibody component
MQATQFPIT

SEQ ID NO: 7 - protein, 125 aa, synthetic construct
  REGION 1..125: note = Description of sequence: Antibody component
QVQLVQSGAEVKKPGASVKVSCKASGYTFTAYYIHWVRQAPGQGLEWMGWINPNSGGTNYAQMFQGRVTMTRDTSISAAYMELSRLSSDDTAVYYCTRGTPRYNWKSYNYGMDVWGQGTTVTVSS

SEQ ID NO: 8 - protein, 112 aa, synthetic construct
  REGION 1..112: note = Description of sequence: Antibody component
DIVVTQTPLSSPVTLGQPASISCRSSQSLVHSDGNTYLSWLQQRPGQPPRLLIYRISNRFSGVPDRFSGSGAGTDFTLKISRVEAEDVGVYYCMQATQFPITFGQGTRLEIK

SEQ ID NO: 9 - protein, 8 aa, synthetic construct
  REGION 1..8: note = Description of sequence: Antibody component
GFTFSSYW

SEQ ID NO: 10 - protein, 8 aa, synthetic construct
  REGION 1..8: note = Description of sequence: Antibody component
MRKDGSEK

SEQ ID NO: 11 - protein, 11 aa, synthetic construct
  REGION 1..11: note = Description of sequence: Antibody component
ARERSTGYFDY

SEQ ID NO: 12 - protein, 6 aa, synthetic construct
  REGION 1..6: note = Description of sequence: Antibody component
QRISTW

SEQ ID NO: 13 - skipped

SEQ ID NO: 14 - protein, 9 aa, synthetic construct
  REGION 1..9: note = Description of sequence: Antibody component
QQYISYTWT

SEQ ID NO: 15 - protein, 118 aa, synthetic construct
  REGION 1..118: note = Description of sequence: Antibody component
EVQLVESGGGLVQPGGSLRLSCAASGFTFSSYWMTWVRQAPGKGLEWVANMRKDGSEKYYVDSVKGRFTISRDNAKNSLYLHMNSLRAEDTAVYYCARERSTGYFDYWGQGTLVIVSS

SEQ ID NO: 16 - protein, 107 aa, synthetic construct
  REGION 1..107: note = Description of sequence: Antibody component
DIQMTQSPSTLSASVGDRVTITCRASQRISTWLAWYQQKAGKAPKLLIYKASSLESGVPSRFSGSGSGTEFTLTIRSLEPDDFATYYCQQYISYTWTFGQGTKVEIK

SEQ ID NO: 17 - protein, 8 aa, synthetic construct
  REGION 1..8: note = Description of sequence: Antibody component
GFTFRSYW

SEQ ID NO: 18 - protein, 8 aa, synthetic construct
  REGION 1..8: note = Description of sequence: Antibody component
IKEDGSEK

SEQ ID NO: 19 - protein, 11 aa, synthetic construct
  REGION 1..11: note = Description of sequence: Antibody component
ARERREGYFDQ

SEQ ID NO: 20 - protein, 6 aa, synthetic construct
  REGION 1..6: note = Description of sequence: Antibody component
QSIGSW

SEQ ID NO: 21 - protein, 9 aa, synthetic construct
  REGION 1..9: note = Description of sequence: Antibody component
QQYKSYSWT

SEQ ID NO: 22 - protein, 118 aa, synthetic construct
  REGION 1..118: note = Description of sequence: Antibody component
EEQLVESGGGLVQPGGSLRFSCEVSGFTFRSYWMSWVRQAPGKGLEWVANIKEDGSEKYYVDSVKGRFTISRDNAKNSLYLQMNSLRAEDTAVYYCARERREGYFDQWGQGTLVTVSS

SEQ ID NO: 23 - protein, 107 aa, synthetic construct
  REGION 1..107: note = Description of sequence: Antibody component
DIQMTQSPSTLSAFVGDRVTITCRASQSIGSWLAWYQQKPGKAPDLLIYKASSLESGVPSRFSGSGSGTEFTLTISSLQPDDFATYYCQQYKSYSWTFGQGTKVEIK

SEQ ID NO: 24 - protein, 8 aa, synthetic construct
  REGION 1..8: note = Description of sequence: Antibody component
GFTFSRFN

SEQ ID NO: 25 - protein, 8 aa, synthetic construct
  REGION 1..8: note = Description of sequence: Antibody component
ISSRSSYI

SEQ ID NO: 26 - protein, 8 aa, synthetic construct
  REGION 1..8: note = Description of sequence: Antibody component
AGGGYFDY

SEQ ID NO: 27 - protein, 11 aa, synthetic construct
  REGION 1..11: note = Description of sequence: Antibody component
QSLLHSNGYNY

SEQ ID NO: 28 - skipped

SEQ ID NO: 29 - protein, 9 aa, synthetic construct
  REGION 1..9: note = Description of sequence: Antibody component
IQALQTPYT

SEQ ID NO: 30 - protein, 115 aa, synthetic construct
  REGION 1..115: note = Description of sequence: Antibody component
EVQLVESGGGLVKPGGSLRLSCAASGFTFSRFNMNWVRQTPGKGLEWVSSISSRSSYIYYADSVKGRFTISRDNAKKSLYLQMNSLRAEDTAVYYCAGGGYFDYWGQGTLVTVSS

SEQ ID NO: 31 - protein, 112 aa, synthetic construct
  REGION 1..112: note = Description of sequence: Antibody component
DIVMTQSPLSLPVTPGEPAAISCRSSQSLLHSNGYNYLDWYLQKPGQSPQLLIYLGSNRASGVPDRFSGSGSGTDFTLKISRVEAEDVGVYYCIQALQTPYTFGQGTKLEIK

SEQ ID NO: 32 - protein, 8 aa, synthetic construct
  REGION 1..8: note = Description of sequence: Antibody component
GYTFTGYY

SEQ ID NO: 33 - protein, 8 aa, synthetic construct
  REGION 1..8: note = Description of sequence: Antibody component
INPYRSGT

SEQ ID NO: 34 - protein, 14 aa, synthetic construct
  REGION 1..14: note = Description of sequence: Antibody component
ARGAGTTFYNAFDI

SEQ ID NO: 35 - protein, 6 aa, synthetic construct
  REGION 1..6: note = Description of sequence: Antibody component
QGINNY

SEQ ID NO: 36 - skipped

SEQ ID NO: 37 - protein, 9 aa, synthetic construct
  REGION 1..9: note = Description of sequence: Antibody component
LQHNRYPFT

SEQ ID NO: 38 - protein, 121 aa, synthetic construct
  REGION 1..121: note = Description of sequence: Antibody component
QVQLVQSGAEVKKPGASVKVSCKAAGYTFTGYYMHWVRQAPGQGLEWMGWINPYRSGTNFAQKFQDRVTMTRDTSISTAYMELSSLSSDDTAIYYCARGAGTTFYNAFDIWGQGTMVTVSS

SEQ ID NO: 39 - protein, 107 aa, synthetic construct
  REGION 1..107: note = Description of sequence: Antibody component
DIQMTQSPSAMSASVGDRVTITCRASQGINNYLAWFQQKPGKVPKRLIYAASSLQSGVPSRFSGSGSGTEFTLTISSLQPEDFATYYCLQHNRYPFTFGPGTKVDIK

SEQ ID NO: 40 - protein, 8 aa, synthetic construct
  REGION 1..8: note = Description of sequence: Antibody component
GFTFSSFW

SEQ ID NO: 41 - protein, 11 aa, synthetic construct
  REGION 1..11: note = Description of sequence: Antibody component
ARERRTGYFDY

SEQ ID NO: 42 - protein, 6 aa, synthetic construct
  REGION 1..6: note = Description of sequence: Antibody component
QSIGSW

SEQ ID NO: 43 - protein, 118 aa, synthetic construct
  REGION 1..118: note = Description of sequence: Antibody component
EVQLVESGGGLVQPGGSLRLSCAASGFTFSSFWMSWVRQAPGKGLEWVANIKEDGSEKYYVDSVKGRFTISRDNAKNSLFLQMNSLRAEDTAVYYCARERRTGYFDYWGQGTLVTVSS

SEQ ID NO: 44 - protein, 107 aa, synthetic construct
  REGION 1..107: note = Description of sequence: Antibody component
DIQMTQSSSTLSASVGDRVTITCRASQSIGSWLAWFQQKPGKAPKLLIYKASSLESGVPSRFSGSGSGTEFTLTISSLQPDDFAIYYCQQYKSYSWTFGQGTKVEIK

SEQ ID NO: 45 - protein, 8 aa, synthetic construct
  REGION 1..8: note = Description of sequence: Antibody component
GFTFSRFW

SEQ ID NO: 46 - protein, 11 aa, synthetic construct
  REGION 1..11: note = Description of sequence: Antibody component
ARERREGYFDY

SEQ ID NO: 47 - protein, 118 aa, synthetic construct
  REGION 1..118: note = Description of sequence: Antibody component
EVQLVESGGGLVQSGGSLRLSCAASGFTFSRFWMTWVRQAPGKGLEWVANIKEDGSEKYYVDSVKGRFTISRDNTKNSLYLQMNSLRAEDTAVYYCARERREGYFDYWGQGTLVTVSS

SEQ ID NO: 48 - protein, 107 aa, synthetic construct
  REGION 1..107: note = Description of sequence: Antibody component
DIQMTQSPSTLSASVGDRVTITCRASQSIGSWLAWYQQKPGKAPKLLIYKASSLESGVPSRFSGSGSGTEFTLTISSLQPDDFATYYCQQYKSYSWTFGHGTKVEIK

SEQ ID NO: 49 - protein, 8 aa, synthetic construct
  REGION 1..8: note = Description of sequence: Antibody component
ISSSSSYI

SEQ ID NO: 50 - protein, 9 aa, synthetic construct
  REGION 1..9: note = Description of sequence: Antibody component
RQALQTPYT

SEQ ID NO: 51 - protein, 115 aa, synthetic construct
  REGION 1..115: note = Description of sequence: Antibody component
EVQLVESGGGLVKPGGSLRLSCAASGFTFSRFNMNWVRQTPGKGLEWVSSISSSSSYIYYADSVKGRFTISRDNAKKSLYLQMNSLRAEDTAVYYCAGGGYFDYWGQGTLVTVSS

SEQ ID NO: 52 - protein, 112 aa, synthetic construct
  REGION 1..112: note = Description of sequence: Antibody component
DIVMTQSPLSLPVTPGEPASISCRSSQILLHSNGYNYLDWYLQKPGQSPQLLIYLGSNRASGVPDRFSGSGSGTDFTLKISRVEAEDVGVFYCRQALQTPYTFGQGTKLEIK

SEQ ID NO: 53 - protein, 6 aa, synthetic construct
  REGION 1..6: note = Description of sequence: Antibody component
QSISSW

SEQ ID NO: 54 - protein, 118 aa, synthetic construct
  REGION 1..118: note = Description of sequence: Antibody component
EVQLVESGRGLVQPGGSLRLSCAASGFTFSSYWMSWVRQAPGKGLEWVANIKEDGSEKYYVDSVKGRFTISRDNAKNSLYLQMNSLRAEDTAVYYCARERREGYFDYWGQGTLVTVSS

SEQ ID NO: 55 - protein, 107 aa, synthetic construct
  REGION 1..107: note = Description of sequence: Antibody component
DIQMTQSPSTLSASVGDRVTITCRASQSISSWLAWYQQKPGKAPDLLIYKASSLESGVPSRFSGSGSGTEFTLTISSLQPDDFATYYCQQYKSYSWTFGQGTKVEIK

SEQ ID NO: 56 - protein, 118 aa, synthetic construct
  REGION 1..118: note = Description of sequence: Antibody component
EVQLVESGGGLVQPGGSLRLSCAASGFTFSSYWMTWVRQAPGKGLEWVANIKEDGSEKYYVDSVKGRFTISRDNAKNSLYVQMNSLRAEDTAIYYCARERREGYFDYWGQGTLVTVSS

SEQ ID NO: 57 - protein, 107 aa, synthetic construct
  REGION 1..107: note = Description of sequence: Antibody component
DIQMTQSPSILSASVGDRATITCRASQRISTWLAWFQQKPGKAPKLLIYKASSLESGVPSRFSGSGSGTEFTLTISSLQPDDFATYYCQQYKSYSWTFGQGTKVEIK

SEQ ID NO: 58 - protein, 6 aa, synthetic construct
  REGION 1..6: note = Description of sequence: Antibody component
QSIGRW

SEQ ID NO: 59 - protein, 118 aa, synthetic construct
  REGION 1..118: note = Description of sequence: Antibody component
EIQLVESGGGLVQPGGSLRLSCAASGFTFSSYWMSWVRQTPGKGLEWVANIKEDGSEKYHVDSVKGRYTISRDNAKNSLYLQMSSLRADDTAVYYCARERREGYFDYWGQGTVVTASS

SEQ ID NO: 60 - protein, 107 aa, synthetic construct
  REGION 1..107: note = Description of sequence: Antibody component
DIQMTQSPSTLSASVGDRVTITCRASQSIGRWLAWYQQKPGKAPDLLIYKASSLESGVPSRFSGGGSGTEFTLTISSLQPDDFATYYCQQYKSYSWTFGQGTKVEVK

SEQ ID NO: 61 - protein, 8 aa, synthetic construct
  REGION 1..8: note = Description of sequence: Antibody component
GYTFTDYY

SEQ ID NO: 62 - protein, 8 aa, synthetic construct
  REGION 1..8: note = Description of sequence: Antibody component
INPYRGAI

SEQ ID NO: 63 - protein, 19 aa, synthetic construct
  REGION 1..19: note = Description of sequence: Antibody component
ARDGGYCSGGSCYKGAFDI

SEQ ID NO: 64 - skipped

SEQ ID NO: 65 - protein, 9 aa, synthetic construct
  REGION 1..9: note = Description of sequence: Antibody component
MQATHFPHT

SEQ ID NO: 66 - protein, 126 aa, synthetic construct
  REGION 1..126: note = Description of sequence: Antibody component
QVQLVQSGAEVKKPGASVKVSCKASGYTFTDYYMHWVRQSPGQGLEWMGWINPYRGAINYAQMFQGRVTMTRDTSISTAYMELSRLNSDDTAVYYCARDGGYCSGGSCYKGAFDIWGQGTMVTVSS

SEQ ID NO: 67 - protein, 112 aa, synthetic construct
  REGION 1..112: note = Description of sequence: Antibody component
DIVMTQTPLSSPVTLGQPASISCRSSQSLVHSDGNTYLSWLQQRPGQPPRLLIYKISNRFSGVPDRFSGSGAGTDFTLKISRVEPEDVGVYYCMQATHFPHTFGQGTRLEIK

SEQ ID NO: 68 - protein, 8 aa, synthetic construct
  REGION 1..8: note = Description of sequence: Antibody component
GFTFKKYN

SEQ ID NO: 69 - protein, 8 aa, synthetic construct
  REGION 1..8: note = Description of sequence: Antibody component
ISSSSTYI

SEQ ID NO: 70 - protein, 115 aa, synthetic construct
  REGION 1..115: note = Description of sequence: Antibody component
EVQVVESGGGLVKPGGSLRLSCVASGFTFKKYNMNWVRQAPGKGLEWVSSISSSSTYIYYADSVKGRFTISRDNAKNSLNLQMNSLRAEDTAVYYCAGGGYFDYWGQGTLVTVSS

SEQ ID NO: 71 - protein, 193 aa, synthetic construct
  REGION 1..193: note = Description of sequence: IL18 polypeptide component
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 72 - protein, 169 aa, synthetic construct
  REGION 1..169: note = Description of sequence: IL36 polypeptide component
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 73 - protein, 270 aa, synthetic construct
  REGION 1..270: note = Description of sequence: IL33 polypeptide component
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 74 - DNA, 123 bp, synthetic construct
  misc_feature 1..123: note = Description of sequence: Nucleotide coding sequence for CD28 polypeptide component
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 75 - protein, 19 aa, synthetic construct
  REGION 1..19: note = Description of sequence: Linker
GGGGSGGGGSGGGSGGGGS

SEQ ID NO: 76 - protein, 15 aa, synthetic construct
  REGION 1..15: note = Description of sequence: Linker
GGGGSGGGGSGGGGS

SEQ ID NO: 77 - protein, 24 aa, synthetic construct
  REGION 1..24: note = Description of sequence: Linker
GGGGSGGGGSGGGGSGGGSGGGGS

SEQ ID NO: 78 - protein, 29 aa, synthetic construct
  REGION 1..29: note = Description of sequence: Linker
GGGGSGGGGSGGGGSGGGGSGGGSGGGGS

SEQ ID NO: 79 - protein, 5 aa, synthetic construct
  REGION 1..5: note = Description of sequence: Linker
GGGGS

SEQ ID NO: 80 - protein, 10 aa, synthetic construct
  REGION 1..10: note = Description of sequence: Linker
GGGGSGGGGS

SEQ ID NO: 81 - protein, 207 aa, synthetic construct
  REGION 1..207: note = Description of sequence: Cytoplasmic domain
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 82 - protein, 330 aa, synthetic construct [sequence omitted]

SEQ ID NO: 83 - protein, 326 aa, synthetic construct
  REGION 1..326: note = Description of sequence: Heavy chain constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 84 - protein, 377 aa, synthetic construct
  REGION 1..377: note = Description of sequence: Heavy chain constant region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 85 - protein, 327 aa, synthetic construct
  REGION 1..327: note = Description of sequence: Heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 107 aa, synthetic construct [sequence omitted]

SEQ ID NO: 87 - protein, 8 aa, synthetic construct
  REGION 1..8: note = Description of sequence: Antibody component
GFTFSTYA

SEQ ID NO: 88 - protein, 8 aa, synthetic construct
  REGION 1..8: note = Description of sequence: Antibody component
ISGRGGST

SEQ ID NO: 89 - protein, 14 aa, synthetic construct
  REGION 1..14: note = Description of sequence: Antibody component
AGRGDYYYYYGMDV

SEQ ID NO: 90 - protein, 11 aa, synthetic construct
  REGION 1..11: note = Description of sequence: Antibody component
QSLVYSDGNTY

SEQ ID NO: 91 - skipped

SEQ ID NO: 92 - protein, 9 aa, synthetic construct
  REGION 1..9: note = Description of sequence: Antibody component
MQSTQFPHT

SEQ ID NO: 93 - protein, 121 aa, synthetic construct
  REGION 1..121: note = Description of sequence: Antibody component
EVQLLESGGGLVQPGGSLRLSCAASGFTFSTYAMSWVRQAPGKGLEWVSAISGRGGSTYYTDSVKGRFTISRDNSKNTVSLQMNSLRAEDTAVYYCAGRGDYYYYYGMDVWGQGTTVTVSA

SEQ ID NO: 94 - protein, 112 aa, synthetic construct
  REGION 1..112: note = Description of sequence: Antibody component
DIVMTQSPLSSPVTLGQPASFSCRSSQSLVYSDGNTYLSWLQQRPGQPPRLLIYKISNRFSGVPDRFSGSGAGTDFTLKISRVEAEDVGVYYCMQSTQFPHTFGQGTKLEIK

SEQ ID NO: 95 - protein, 220 aa, synthetic construct
  REGION 1..220: note = Description of sequence: CD28 polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 96 - protein, 164 aa, synthetic construct
  REGION 1..164: note = Description of sequence: Native CD3-gamma
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 97 - protein, 112 aa, synthetic construct
  REGION 1..112: note = Description of sequence: CD3-gamma polypeptide component
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 255 aa, synthetic construct
  REGION 1..255: note = Description of sequence: 4-1BB polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 99 - protein, 22 aa, Homo sapiens
  REGION 1..22: note = Description of sequence: Native ITAM1
QNQLYNELNLGRREEYDVLDKR

SEQ ID NO: 100 - DNA, 66 bp, Homo sapiens
  misc_feature 1..66: note = Description of sequence: Nucleotide sequence of native ITAM1
  source 1..66: mol_type = other DNA
cagaaccagctctataacgagctcaatctaggacgaagagaggagtacgatgttttggacaagaga

SEQ ID NO: 101 - protein, 22 aa, synthetic construct
  REGION 1..22: note = Description of sequence: ITAM1 varient
QNQLFNELNLGRREEFDVLDKR

SEQ ID NO: 102 - DNA, 66 bp, synthetic construct
  misc_feature 1..66: note = Description of sequence: Nucleotide sequence of ITAM1 Variant
  source 1..66: mol_type = other DNA
cagaaccagctctttaacgagctcaatctaggacgaagagaggagttcgatgttttggacaagaga

SEQ ID NO: 103 - protein, 23 aa, Homo sapiens
  REGION 1..23: note = Description of sequence: Native ITAM2
QEGLYNELQKDKMAEAYSEIGMK

SEQ ID NO: 104 - DNA, 69 bp, Homo sapiens
  misc_feature 1..69: note = Description of sequence: Nucleotide sequence of native ITAM2
  source 1..69: mol_type = other DNA
caggaaggcctgtacaatgaactgcagaaagataagatggcggaggcctacagtgagattgggatgaaa

SEQ ID NO: 105 - protein, 23 aa, synthetic construct
  REGION 1..23: note = Description of sequence: ITAM2 varient
QEGLFNELQKDKMAEAFSEIGMK

SEQ ID NO: 106 - DNA, 69 bp, synthetic construct
  misc_feature 1..69: note = Description of sequence: Nucleotide sequence of ITAM2 Variant
  source 1..69: mol_type = other DNA
caggaaggcctgttcaatgaactgcagaaagataagatggcggaggccttcagtgagattgggatgaaa

SEQ ID NO: 107 - protein, 22 aa, Homo sapiens
  REGION 1..22: note = Description of sequence: Native ITAM3
HDGLYQGLSTATKDTYDALHMQ

SEQ ID NO: 108 - DNA, 66 bp, Homo sapiens
  misc_feature 1..66: note = Description of sequence: Nucleotide sequence of native ITAM3
  source 1..66: mol_type = other DNA
cacgatggcctttaccagggtctcagtacagccaccaaggacacctacgacgcccttcacatgcag

SEQ ID NO: 109 - protein, 22 aa, synthetic construct
  REGION 1..22: note = Description of sequence: ITAM3 varient
HDGLFQGLSTATKDTFDALHMQ

SEQ ID NO: 110 - DNA, 66 bp, synthetic construct
  misc_feature 1..66: note = Description of sequence: Nucleotide sequence of ITAM3 variant
  source 1..66: mol_type = other DNA
cacgatggccttttccaggggctcagtacagccaccaaggacaccttcgacgcccttcacatgcag

SEQ ID NO: 111 - protein, 112 aa, synthetic construct
  REGION 1..112: note = Description of sequence: CD3-gamma polypeptide component
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 112 - DNA, 336 bp, synthetic construct
  misc_feature 1..336: note = Description of sequence: Nucleotide coding sequence for CD3-gamma polypeptide component
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 113 - protein, 21 aa, synthetic construct
  REGION 1..21: note = Description of sequence: Truncated CD8 Signal peptide
MALPVTALLLPLALLLHAARP

SEQ ID NO: 114 - DNA, 63 bp, synthetic construct
  misc_feature 1..63: note = Description of sequence: Nucleotide sequences for truncated CD8 Signal peptide
  source 1..63: mol_type = other DNA
atggcgttgccggtgaccgcgcttttgctccctctggccctgctcttgcacgccgcacgaccg

SEQ ID NO: 115 - protein, 8 aa, synthetic construct
  REGION 1..8: note = Description of sequence: Linker
AAAGGGGS

SEQ ID NO: 116 - protein, 8 aa, synthetic construct
  REGION 1..8: note = Description of sequence: Antibody component
GFTFSTYG

SEQ ID NO: 117 - protein, 8 aa, synthetic construct
  REGION 1..8: note = Description of sequence: Antibody component
IWYDGSNT

SEQ ID NO: 118 - protein, 20 aa, synthetic construct
  REGION 1..20: note = Description of sequence: Antibody component
ARGRGYSAQGNRNRAYYFDY

SEQ ID NO: 119 - protein, 6 aa, synthetic construct
  REGION 1..6: note = Description of sequence: Antibody component
QSVSSN

SEQ ID NO: 120 - skipped

SEQ ID NO: 121 - protein, 10 aa, synthetic construct
  REGION 1..10: note = Description of sequence: Antibody component
QQYNDRPPYT

SEQ ID NO: 122 - protein, 126 aa, synthetic construct
  REGION 1..126: note = Description of sequence: Antibody component
QVQLVESGGGVVQPGRSLRLSCAASGFTFSTYGMHWVRQAPGKGLEWVAVIWYDGSNTYYADSVKGRFTISRDNSKNTLYLQIKSLRAEDTAVYYCARGRGYSAQGNRNRAYYFDYWGQGTLVTVS

SEQ ID NO: 123 - protein, 113 aa, synthetic construct
  REGION 1..113: note = Description of sequence: Antibody component
EIVMTQSPATLSVSPGERVILSCRASQSVSSNLAWYQQKPGQPPRLLIYGAFTRVTGVPARFSGSGSGTEFTLTISSLQSEDFAVYYCQQYNDRPPYTFGQGTKLEIKRAVDQ

SEQ ID NO: 124 - protein, 254 aa, synthetic construct
  REGION 1..254: note = Description of sequence: Antibody component
QVQLVESGGGVVQPGRSLRLSCAASGFTFSTYGMHWVRQAPGKGLEWVAVIWYDGSNTYYADSVKGRFTISRDNSKNTLYLQIKSLRAEDTAVYYCARGRGYSAQGNRNRAYYFDYWGQGTLVTVSGGGGSGGGGSGGGGSEIVMTQSPATLSVSPGERVILSCRASQSVSSNLAWYQQKPGQPPRLLIYGAFTRVTGVPARFSGSGSGTEFTLTISSLQSEDFAVYYCQQYNDRPPYTFGQGTKLEIKRAVDQ

SEQ ID NO: 125 - DNA, 762 bp, synthetic construct
  misc_feature 1..762: note = Description of sequence: Nucleotide sequences for antibody component
  source 1..762: mol_type = other DNA
gaggtgcagctggtggagtctgggggaggcgtggtccagcctgggaggtccctgagactctcctgtgcagcgtctggattcaccttcagtacctatggcatgcactgggtccgccaggctccaggcaaggggcttgagtgggtggcagttatatggtatgatggaagtaatacatactatgcagactccgtgaagggccgattcaccatctccagagacaattccaagaacacactgtatctgcaaataaagagcctgagagccgaggacacggctgtctattactgtgcgagaggccgtggatatagtgcccaagggaatcggaatagggcttactactttgactactggggccagggaaccctggtcaccgtctccggtggaggtggatcaggtggaggtggatctggtggaggtggatctgaaatagtgatgacgcagtctccagccaccctgtctgtgtctccaggggaaagggtcatcctctcctgcagggccagtcagagtgttagcagcaacttagcctggtaccagcagaaacctggccagcctcccaggctcctcatctatggtgcattcacgagggtcactggtgtcccagccaggttcagtggcagtgggtctgggacagaattcactctcaccatcagcagcctgcagtctgaagattttgcagtttattactgtcagcagtacaatgaccggcccccgtacacttttggccaggggaccaagctggagatcaaacgggctgtcgaccaa

SEQ ID NO: 126 - protein, 254 aa, synthetic construct
  REGION 1..254: note = Description of sequence: Antibody component
EIVMTQSPATLSVSPGERVILSCRASQSVSSNLAWYQQKPGQPPRLLIYGAFTRVTGVPARFSGSGSGTEFTLTISSLQSEDFAVYYCQQYNDRPPYTFGQGTKLEIKRAVDQGGGGSGGGGSGGGGSQVQLVESGGGVVQPGRSLRLSCAASGFTFSTYGMHWVRQAPGKGLEWVAVIWYDGSNTYYADSVKGRFTISRDNSKNTLYLQIKSLRAEDTAVYYCARGRGYSAQGNRNRAYYFDYWGQGTLVTVS

SEQ ID NO: 127 - DNA, 762 bp, synthetic construct
  misc_feature 1..762: note = Description of sequence: Nucleotide sequences for antibody component
  source 1..762: mol_type = other DNA
gaaatagtgatgacgcagtctccagccaccctgtctgtgtctccaggggaaagggtcatcctctcctgcagggccagtcagagtgttagcagcaacttagcctggtaccagcagaaacctggccagcctcccaggctcctcatctatggtgcattcacgagggtcactggtgtcccagccaggttcagtggcagtgggtctgggacagaattcactctcaccatcagcagcctgcagtctgaagattttgcagtttattactgtcagcagtacaatgaccggcccccgtacacttttggccaggggaccaagctggagatcaaacgggctgtcgaccaaggaggtggaggatctgggggtggaggttctggtgggggtggatctcaggtgcagctggtggagtctgggggaggcgtggtccagccagggcggtccctgagactctcctgtgcagcgtctggattcaccttcagtacctatggcatgcactgggtccgccaggctccaggcaaggggcttgagtgggtggcagttatatggtatgatggaagtaatacatactatgcagactccgtgaagggccgattcaccatctccagagacaattccaagaacacactgtatctgcaaataaagagcctgagagccgaggacacggctgtctattactgtgcgagaggccgtggatatagtgcccaagggaatcggaatagggcttactactttgactactggggccagggaaccctggtcaccgtctcc

SEQ ID NO: 128 - protein, 542 aa, synthetic construct
  REGION 1..542: note = Description of sequence: Bispecific molecule
MALPVTALLLPLALLLHAQVQLVESGGGVVQPGRSLRLSCAASGFTFSTYGMHWVRQAPGKGLEWVAVIWYDGSNTYYADSVKGRFTISRDNSKNTLYLQIKSLRAEDTAVYYCARGRGYSAQGNRNRAYYFDYWGQGTLVTVSGGGGSGGGGSGGGGSEIVMTQSPATLSVSPGERVILSCRASQSVSSNLAWYQQKPGQPPRLLIYGAFTRVTGVPARFSGSGSGTEFTLTISSLQSEDFAVYYCQQYNDRPPYTFGQGTKLEIKRAVDQEQKLISEEDLAAAGGGGSQVQLVQSGAEVKKPGASVKVSCKASGYTFTAYYIHWVRQAPGQGLEWMGWINPNSGGTNYAQMFQGRVTMTRDTSISAAYMELSRLSSDDTAVYYCTRGTPRYNWKSYNYGMDVWGQGTTVTVSSGGGGSGGGGSGGGGSDIVVTQTPLSSPVTLGQPASISCRSSQSLVHSDGNTYLSWLQQRPGQPPRLLIYRISNRFSGVPDRFSGSGAGTDFTLKISRVEAEDVGVYYCMQATQFPITFGQGTRLEIK

SEQ ID NO: 129 - DNA, 1626 bp, synthetic construct
  misc_feature 1..1626: note = Description of sequence: Nucleotide sequence of a bispecific molecule
  source 1..1626: mol_type = other DNA
atggctctcccagtgactgccctactgcttcccctagcgcttctcctgcatgcacaggtgcagctggtggagtctgggggaggcgtggtccagcctgggaggtccctgagactctcctgtgcagcgtctggattcaccttcagtacctatggcatgcactgggtccgccaggctccaggcaaggggcttgagtgggtggcagttatatggtatgatggaagtaatacatactatgcagactccgtgaagggccgattcaccatctccagagacaattccaagaacacactgtatctgcaaataaagagcctgagagccgaggacacggctgtctattactgtgcgagaggccgtggatatagtgcccaagggaatcggaatagggcttactactttgactactggggccagggaaccctggtcaccgtctccggtggaggtggatcaggtggaggtggatctggtggaggtggatctgaaatagtgatgacgcagtctccagccaccctgtctgtgtctccaggggaaagggtcatcctctcctgcagggccagtcagagtgttagcagcaacttagcctggtaccagcagaaacctggccagcctcccaggctcctcatctatggtgcattcacgagggtcactggtgtcccagccaggttcagtggcagtgggtctgggacagaattcactctcaccatcagcagcctgcagtctgaagattttgcagtttattactgtcagcagtacaatgaccggcccccgtacacttttggccaggggaccaagctggagatcaaacgggctgtcgaccaagaacagaaactgatctctgaagaagacctggcggccgcaggtgggggaggaagtcaggtgcagctggtgcagtctggggctgaggtgaagaagcctggggcctcagtgaaggtctcctgcaaggcttctggatacaccttcaccgcctactatatacactgggtgcgacaggcccctggacaaggacttgagtggatgggatggatcaaccctaacagtggtggcacaaactatgcacagatgtttcagggcagggtcaccatgaccagggacacgtccatcagcgcagcctacatggagctgagcaggctgagctctgacgacacggccgtatattactgtacgagaggaactccccgttataactggaagtcctacaactacggtatggacgtctggggccaagggaccacggtcaccgtctcctcaggagggggtggttctggaggtggtgggtcagggggggggggctccgatattgtggtgacccagactccactctcctcacctgtcactcttggacaaccggcctccatctcctgcaggtctagtcaaagcctcgtacacagtgatggaaacacctacttgagttggcttcagcagaggccaggccagcctccaagactcctaatttataggatttctaaccgtttctctggggtcccagacagattcagtggcagtggggctgggacagatttcacactgaagatcagcagggtggaagctgaggatgtcggggtttattattgcatgcaagctacacaatttcctatcaccttcggccaagggacacgactggagattaaa

SEQ ID NO: 130 - protein, 542 aa, synthetic construct
  REGION 1..542: note = Description of sequence: Bispecific molecule
MALPVTALLLPLALLLHAEIVMTQSPATLSVSPGERVILSCRASQSVSSNLAWYQQKPGQPPRLLIYGAFTRVTGVPARFSGSGSGTEFTLTISSLQSEDFAVYYCQQYNDRPPYTFGQGTKLEIKRAVDQGGGGSGGGGSGGGGSQVQLVESGGGVVQPGRSLRLSCAASGFTFSTYGMHWVRQAPGKGLEWVAVIWYDGSNTYYADSVKGRFTISRDNSKNTLYLQIKSLRAEDTAVYYCARGRGYSAQGNRNRAYYFDYWGQGTLVTVSEQKLISEEDLAAAGGGGSQVQLVQSGAEVKKPGASVKVSCKASGYTFTAYYIHWVRQAPGQGLEWMGWINPNSGGTNYAQMFQGRVTMTRDTSISAAYMELSRLSSDDTAVYYCTRGTPRYNWKSYNYGMDVWGQGTTVTVSSGGGGSGGGGSGGGGSDIVVTQTPLSSPVTLGQPASISCRSSQSLVHSDGNTYLSWLQQRPGQPPRLLIYRISNRFSGVPDRFSGSGAGTDFTLKISRVEAEDVGVYYCMQATQFPITFGQGTRLEIK

SEQ ID NO: 131 - DNA, 1626 bp, synthetic construct
  misc_feature 1..1626: note = Description of sequence: Nucleotide sequence of a bispecific molecule
  source 1..1626: mol_type = other DNA
atggctctcccagtgactgccctactgcttcccctagcgcttctcctgcatgcagaaatagtgatgacgcagtctccagccaccctgtctgtgtctccaggggaaagggtcatcctctcctgcagggccagtcagagtgttagcagcaacttagcctggtaccagcagaaacctggccagcctcccaggctcctcatctatggtgcattcacgagggtcactggtgtcccagccaggttcagtggcagtgggtctgggacagaattcactctcaccatcagcagcctgcagtctgaagattttgcagtttattactgtcagcagtacaatgaccggcccccgtacacttttggccaggggaccaagctggagatcaaacgggctgtcgaccaaggaggtggaggatctgggggtggaggttctggtgggggtggatctcaggtgcagctggtggagtctgggggaggcgtggtccagccagggcggtccctgagactctcctgtgcagcgtctggattcaccttcagtacctatggcatgcactgggtccgccaggctccaggcaaggggcttgagtgggtggcagttatatggtatgatggaagtaatacatactatgcagactccgtgaagggccgattcaccatctccagagacaattccaagaacacactgtatctgcaaataaagagcctgagagccgaggacacggctgtctattactgtgcgagaggccgtggatatagtgcccaagggaatcggaatagggcttactactttgactactggggccagggaaccctggtcaccgtctccgaacagaaactgatctctgaagaagacctggcggccgcaggtgggggaggaagtcaggtgcagctggtgcagtctggggctgaggtgaagaagcctggggcctcagtgaaggtctcctgcaaggcttctggatacaccttcaccgcctactatatacactgggtgcgacaggcccctggacaaggacttgagtggatgggatggatcaaccctaacagtggtggcacaaactatgcacagatgtttcagggcagggtcaccatgaccagggacacgtccatcagcgcagcctacatggagctgagcaggctgagctctgacgacacggccgtatattactgtacgagaggaactccccgttataactggaagtcctacaactacggtatggacgtctggggccaagggaccacggtcaccgtctcctcaggagggggtggttctggaggtggtgggtcagggggggggggctccgatattgtggtgacccagactccactctcctcacctgtcactcttggacaaccggcctccatctcctgcaggtctagtcaaagcctcgtacacagtgatggaaacacctacttgagttggcttcagcagaggccaggccagcctccaagactcctaatttataggatttctaaccgtttctctggggtcccagacagattcagtggcagtggggctgggacagatttcacactgaagatcagcagggtggaagctgaggatgtcggggtttattattgcatgcaagctacacaatttcctatcaccttcggccaagggacacgactggagattaaa

SEQ ID NO: 132 - protein, 8 aa, synthetic construct
  REGION 1..8: note = Description of sequence: Antibody component
GFTFSDYQ

SEQ ID NO: 133 - protein, 8 aa, synthetic construct
  REGION 1..8: note = Description of sequence: Antibody component
IQGGGGST

SEQ ID NO: 134 - protein, 15 aa, synthetic construct
  REGION 1..15: note = Description of sequence: Antibody component
AREMWRGDYYSGMDV

SEQ ID NO: 135 - protein, 12 aa, synthetic construct
  REGION 1..12: note = Description of sequence: Antibody component
QSVLDSYNNENN

SEQ ID NO: 136 - skipped

SEQ ID NO: 137 - protein, 9 aa, synthetic construct
  REGION 1..9: note = Description of sequence: Antibody component
QQYTSEPIT

SEQ ID NO: 138 - protein, 122 aa, synthetic construct
  REGION 1..122: note = Description of sequence: Antibody component
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 139 - protein, 113 aa, synthetic construct
  REGION 1..113: note = Description of sequence: Antibody component
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 140 - protein, 259 aa, synthetic construct
  REGION 1..259: note = Description of sequence: Antibody component
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 141 - DNA, 777 bp, synthetic construct
  misc_feature 1..777: note = Description of sequence: Nucleotide sequences for antibody component
  source 1..777: mol_type = other DNA
gaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaa

SEQ ID NO: 142 - protein, 254 aa, synthetic construct
  REGION 1..254: note = Description of sequence: Antibody component
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 143 - DNA, 762 bp, synthetic construct
  misc_feature 1..762: note = Description of sequence: Nucleotide sequences for antibody component
  source 1..762: mol_type = other DNA
gacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctca

SEQ ID NO: 144 - protein, 547 aa, synthetic construct
  REGION 1..547: note = Description of sequence: Bispecific molecule
MALPVTALLLPLALLLHAEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKEQKLISEEDLAAAGGGGSQVQLVQSGAEVKKPGASVKVSCKASGYTFTAYYIHWVRQAPGQGLEWMGWINPNSGGTNYAQMFQGRVTMTRDTSISAAYMELSRLSSDDTAVYYCTRGTPRYNWKSYNYGMDVWGQGTTVTVSSGGGGSGGGGSGGGGSDIVVTQTPLSSPVTLGQPASISCRSSQSLVHSDGNTYLSWLQQRPGQPPRLLIYRISNRFSGVPDRFSGSGAGTDFTLKISRVEAEDVGVYYCMQATQFPITFGQGTRLEIK

SEQ ID NO: 145 - DNA, 1641 bp, synthetic construct
  misc_feature 1..1641: note = Description of sequence: Nucleotide sequence of a bispecific molecule
  source 1..1641: mol_type = other DNA
atggctctcccagtgactgccctactgcttcccctagcgcttctcctgcatgcagaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctcaggtggtggtggttcaggtggtggtggttctggagggggcggttctggcggcggctccggtggtggtggatccgacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaagaacagaaactgatctctgaagaagacctggcggccgcaggtgggggaggaagtcaggtgcagctggtgcagtctggggctgaggtgaagaagcctggggcctcagtgaaggtctcctgcaaggcttctggatacaccttcaccgcctactatatacactgggtgcgacaggcccctggacaaggacttgagtggatgggatggatcaaccctaacagtggtggcacaaactatgcacagatgtttcagggcagggtcaccatgaccagggacacgtccatcagcgcagcctacatggagctgagcaggctgagctctgacgacacggccgtatattactgtacgagaggaactccccgttataactggaagtcctacaactacggtatggacgtctggggccaagggaccacggtcaccgtctcctcaggagggggtggttctggaggtggtgggtcagggggggggggctccgatattgtggtgacccagactccactctcctcacctgtcactcttggacaaccggcctccatctcctgcaggtctagtcaaagcctcgtacacagtgatggaaacacctacttgagttggcttcagcagaggccaggccagcctccaagactcctaatttataggatttctaaccgtttctctggggtcccagacagattcagtggcagtggggctgggacagatttcacactgaagatcagcagggtggaagctgaggatgtcggggtttattattgcatgcaagctacacaatttcctatcaccttcggccaagggacacgactggagattaaa

(19) United States
(12) Patent Application Publication
(10) Pub. No.: US 2026/0146089 A1
(43) Pub. Date: May 28, 2026

(54) ANTIBODIES TARGETING CD3 AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(72) Inventors: Anthony Daniyan, Somers, NY (US); Ivo Lorenz, New York, NY (US); David Andrew, Sandy Hook, CT (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US); TRI-INSTITUTIONAL THERAPEUTICS DISCOVERY INSTITUTE, INC., New York, NY (US)

(21) Appl. No.: 19/329,141

(22) Filed: Sep. 15, 2025

Related U.S. Application Data
(63) Continuation of application No. PCT/US2024/019834, filed on Mar. 14, 2024.
(60) Provisional application No. 63/490,940, filed on Mar. 17, 2023.

(51) Int. Cl. C07K 16/28; A61K 47/68; A61P 35/02; C07K 14/54; C07K 16/44

(52) U.S. Cl. CPC C07K 16/2809; A61K 47/6879; A61P 35/02; C07K 14/54; C07K 16/2803; C07K 16/2851; C07K 16/44; C07K 2317/31; C07K 2317/565; C07K 2319/00

(57) ABSTRACT

The presently disclosed subject matter provides antibodies or antigen-binding fragments thereof that bind to CD3 and methods of using such antibodies or antigen-binding fragments thereof.

### CROSS-REFERENCE TO RELATED APPLICATIONS

The present application is a continuation application of International Patent Application No. PCT/US2024/019834, filed Mar. 14, 2024, which claims priority to U.S. Provisional Patent Application No. 63/490,940, filed Mar. 17, 2023, the content of each of which is incorporated by reference in its entirety, and to each of which priority is claimed.

### SEQUENCE LISTING

The present application contains a Sequence Listing which has been electronically submitted and is hereby incorporated by reference in its entirety. Said Sequence Listing, created on Sep. 15, 2025, is named 0727341856_ST26.xml and is 194,580 bytes in size.

### TECHNICAL FIELD

The presently disclosed subject matter relates to antibodies that bind to CD3, methods of using such antibodies.

### BACKGROUND

Immunotherapy of cancer with CD3-bispecific antibodies is an approved therapeutic option for some hematological cancers and is under clinical investigation for solid cancers. However, the treatment of solid tumors faces more pronounced hurdles, such as increased on-target off-tumor toxicities, sparse T-cell infiltration, and impaired T-cell quality due to the presence of an immunosuppressive tumor microenvironment, which affects the safety and limits the efficacy of CD3-bispecific antibody therapy. Hence, there is a critical need for novel CD3 antibodies and methods of using such agents.

### SUMMARY

The presently disclosed subject matter provides antibodies or antigen-binding fragments thereof that specifically bind to CD38, multi-specific molecules that specifically bind to CD38, and methods of using these antibodies or antigen-binding fragments thereof.

In certain non-limiting embodiments, the presently disclosed subject matter provides an anti-CD3 antibody or an antigen-binding fragment thereof, comprising:
(a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, a CDR3 comprising the amino acid sequence set forth in SEQ ID NO: 3 or a conservative modification thereof; and a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof;
(b) a heavy chain variable region comprising CDR1 comprising the amino acid sequence set forth in SEQ ID NO: 9 or a conservative modification thereof, a CDR2 comprising an amino acid sequence set forth in SEQ ID NO: 10 or a conservative modification thereof, a CDR3 comprising the amino acid sequence set forth in SEQ ID NO: 11 or a conservative modification thereof; and a light chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof;
(c) a heavy chain variable region comprising CDR1 comprising the amino acid sequence set forth in SEQ ID NO: 17 or a conservative modification thereof, a CDR2 comprising an amino acid sequence set forth in SEQ ID NO: 18 or a conservative modification thereof, a CDR3 comprising the amino acid sequence set forth in SEQ ID NO: 19 or a conservative modification thereof; and a light chain variable region comprising a CDR1 comprising the amino acid sequence set forth in SEQ ID NO: 20 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof;
(d) a heavy chain variable region comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, a CDR3 comprising the amino acid sequence set forth in SEQ ID NO: 26 or a conservative modification thereof; and a light chain variable region comprising a CDR1 comprising the amino acid sequence set forth in SEQ ID NO: 27 or a conservative modification thereof, a CDR2 comprising the amino acid sequence set forth in SEQ ID NO: 28 or a conservative modification thereof, and a CDR3 comprising the amino acid sequence set forth in SEQ ID NO: 29 or a conservative modification thereof;
(e) a heavy chain variable region comprising CDR1 comprising the amino acid sequence set forth in SEQ ID NO: 32 or a conservative modification thereof, a CDR2 comprising an amino acid sequence set forth in SEQ ID NO: 33 or a conservative modification thereof, a CDR3 comprising the amino acid sequence set forth in SEQ ID NO: 34 or a conservative modification thereof; and a light chain variable region comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 37 or a conservative modification thereof;
(f) a heavy chain variable region comprising a CDR1 comprising the amino acid sequence set forth in SEQ ID NO: 40 or a conservative modification thereof, a CDR2 comprising the amino acid sequence set forth in SEQ ID NO: 18 or a conservative modification thereof, a CDR3 comprising the amino acid sequence set forth in SEQ ID NO: 41 or a conservative modification thereof; and a light chain variable region comprising a CDR1 comprising the amino acid sequence set forth in SEQ ID NO: 42 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof;
(g) a heavy chain variable region comprising CDR1 comprising the amino acid sequence set forth in SEQ ID NO: 45 or a conservative modification thereof, a CDR2 comprising an amino acid sequence set forth in SEQ ID NO: 18 or a conservative modification thereof, a CDR3 comprising the amino acid sequence set forth in SEQ ID NO: 46 or a conservative modification thereof; and a light chain variable region comprising a CDR1 comprising the amino acid sequence set forth in SEQ ID NO: 20 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof;
(h) a heavy chain variable region comprising CDR1 comprising the amino acid sequence set forth in SEQ ID NO: 24 or a conservative modification thereof, a CDR2 comprising an amino acid sequence set forth in SEQ ID NO: 49 or a conservative modification thereof, a CDR3 comprising the amino acid sequence set forth in SEQ ID NO: 26 or a conservative modification thereof; and a light chain variable region comprising a CDR1 comprising the amino acid sequence set forth in SEQ ID NO: 20 or a conservative modification thereof, a CDR2 comprising the amino acid sequence set forth in SEQ ID NO: 28 or a conservative modification thereof, and a CDR3 comprising the amino acid sequence set forth in SEQ ID NO: 50 or a conservative modification thereof;
(i) a heavy chain variable region comprising a CDR1 comprising the amino acid sequence set forth in SEQ ID NO: 9 or a conservative modification thereof, a CDR2 comprising the amino acid sequence set forth in SEQ ID NO: 18 or a conservative modification thereof, a CDR3 comprising the amino acid sequence set forth in SEQ ID NO: 46 or a conservative modification thereof; and a light chain variable region comprising a CDR1 comprising the amino acid sequence set forth in SEQ ID NO: 53 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof;
(j) a heavy chain variable region comprising CDR1 comprising the amino acid sequence set forth in SEQ ID NO: 9 or a conservative modification thereof, a CDR2 comprising an amino acid sequence set forth in SEQ ID NO: 18 or a conservative modification thereof, a CDR3 comprising the amino acid sequence set forth in SEQ ID NO: 46 or a conservative modification thereof; and a light chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof;
(k) a heavy chain variable region comprising CDR1 comprising the amino acid sequence set forth in SEQ ID NO: 9 or a conservative modification thereof, a CDR2 comprising an amino acid sequence set forth in SEQ ID NO: 18 or a conservative modification thereof, a CDR3 comprising the amino acid sequence set forth in SEQ ID NO: 4 or a conservative modification thereof 6; and a light chain variable region comprising a CDR1 comprising the amino acid sequence set forth in SEQ ID NO: 58 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof; or
(l) a heavy chain variable region comprising a CDR1 comprising the amino acid sequence set forth in SEQ ID NO: 61 or a conservative modification thereof, a CDR2 comprising the amino acid sequence set forth in SEQ ID NO: 62 or a conservative modification thereof, a CDR3 comprising the amino acid sequence set forth in SEQ ID NO: 63 or a conservative modification thereof; and a light chain variable region comprising a CDR1 comprising the amino acid sequence set forth in SEQ ID NO: 4 or a conservative modification thereof, a CDR2 comprising the amino acid sequence set forth in SEQ ID NO: 64 or a conservative modification thereof, and a CDR3 comprising the amino acid sequence set forth in SEQ ID NO: 65 or a conservative modification thereof.

In certain embodiments, the anti-CD3 antibody or an antigen-binding fragment thereof comprises:
(a) a heavy chain variable region comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising CDR1 comprising the amino acid sequence set forth in SEQ ID NO: 9, a CDR2 comprising an amino acid sequence set forth in SEQ ID NO: 10, a CDR3 comprising the amino acid sequence set forth in SEQ ID NO: 11; and a light chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14;
(c) a heavy chain variable region comprising CDR1 comprising the amino acid sequence set forth in SEQ ID NO: 17, a CDR2 comprising an amino acid sequence set forth in SEQ ID NO: 18, a CDR3 comprising the amino acid sequence set forth in SEQ ID NO: 19; and a light chain variable region comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21;
(d) a heavy chain variable region comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a light chain variable region comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29;
(e) a heavy chain variable region comprising CDR1 comprising the amino acid sequence set forth in SEQ ID NO: 32, a CDR2 comprising an amino acid sequence set forth in SEQ ID NO: 33, a CDR3 comprising the amino acid sequence set forth in SEQ ID NO: 34; and a light chain variable region comprising a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 37;
(f) a heavy chain variable region comprising a CDR1 comprising the amino acid sequence set forth in SEQ ID NO: 40, a CDR2 comprising the amino acid sequence set forth in SEQ ID NO: 18, a CDR3 comprising the amino acid sequence set forth in SEQ ID NO: 41; and a light chain variable region comprising a CDR1 comprising the amino acid sequence set forth in SEQ ID NO: 42, a CDR2 comprising the amino acid sequence set forth in SEQ ID NO: 13, and a CDR3 comprising the amino acid sequence set forth in SEQ ID NO: 21;
(g) a heavy chain variable region comprising CDR1 comprising the amino acid sequence set forth in SEQ ID NO: 45, a CDR2 comprising an amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21;
(h) a heavy chain variable region comprising CDR1 comprising the amino acid sequence set forth in SEQ ID NO: 24, a CDR2 comprising an amino acid sequence set forth in SEQ ID NO: 49, a CDR3 comprising the amino acid sequence set forth in SEQ ID NO: 26; and a light chain variable region comprising a CDR1 comprising the amino acid sequence set forth in SEQ ID NO: 20, a CDR2 comprising the amino acid sequence set forth in SEQ ID NO: 28, and a CDR3 comprising the amino acid sequence set forth in SEQ ID NO: 50;
(i) a heavy chain variable region comprising a CDR1 comprising the amino acid sequence set forth in SEQ ID NO: 9, a CDR2 comprising the amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 53, a CDR2 comprising the amino acid sequence set forth in SEQ ID NO: 13, and a CDR3 comprising the amino acid sequence set forth in SEQ ID NO: 21;
(j) a heavy chain variable region comprising CDR1 comprising the amino acid sequence set forth in SEQ ID NO: 9, a CDR2 comprising an amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 21;
(k) a heavy chain variable region comprising CDR1 comprising the amino acid sequence set forth in SEQ ID NO: 9, a CDR2 comprising an amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 58, a CDR2 comprising the amino acid sequence set forth in SEQ ID NO: 13, and a CDR3 comprising the amino acid sequence set forth in SEQ ID NO: 21; or
(l) a heavy chain variable region comprising a CDR1 comprising the amino acid sequence set forth in SEQ ID NO: 61, a CDR2 comprising the amino acid sequence set forth in SEQ ID NO: 62, a CDR3 comprising the amino acid sequence set forth in SEQ ID NO: 63; and a light chain variable region comprising a CDR1 comprising the amino acid sequence set forth in SEQ ID NO: 4, a CDR2 comprising the amino acid sequence set forth in SEQ ID NO: 64, and a CDR3 comprising the amino acid sequence set forth in SEQ ID NO: 65.

In certain embodiments, the anti-CD3 antibody or an antigen-binding fragment thereof comprises:
(a) a heavy chain variable region comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising CDR1 comprising the amino acid sequence set forth in SEQ ID NO: 9, a CDR2 comprising an amino acid sequence set forth in SEQ ID NO: 10, a CDR3 comprising the amino acid sequence set forth in SEQ ID NO: 11; and a light chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14;
(c) a heavy chain variable region comprising CDR1 comprising the amino acid sequence set forth in SEQ ID NO: 17, a CDR2 comprising an amino acid sequence set forth in SEQ ID NO: 18, a CDR3 comprising the amino acid sequence set forth in SEQ ID NO: 19; and a light chain variable region comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21; or
(d) a heavy chain variable region comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a light chain variable region comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29.

In certain embodiments, the anti-CD3 antibody or an antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 15, SEQ ID NO: 22, SEQ ID NO: 30, SEQ ID NO: 38, SEQ ID NO: 43, SEQ ID NO: 47, SEQ ID NO: 51, SEQ ID NO: 54, SEQ ID NO: 56, SEQ ID NO: 59, SEQ ID NO: 66, or SEQ ID NO: 70.

In certain embodiments, the anti-CD3 antibody or an antigen-binding fragment thereof comprises a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 16, SEQ ID NO: 23, SEQ ID NO: 31, SEQ ID NO: 39, SEQ ID NO: 44, SEQ ID NO: 48, SEQ ID NO: 52, SEQ ID NO: 55, SEQ ID NO: 57, SEQ ID NO: 60, or SEQ ID NO: 67.

In certain embodiments, the anti-CD3 antibody or an antigen-binding fragment thereof comprises:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 15, SEQ ID NO: 22, SEQ ID NO: 30, SEQ ID NO: 38, SEQ ID NO: 43, SEQ ID NO: 47, SEQ ID NO: 51, SEQ ID NO: 54, SEQ ID NO: 56, SEQ ID NO: 59, SEQ ID NO: 66, or SEQ ID NO: 70; or
(b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 8, SEQ ID NO: 16, SEQ ID NO: 23, SEQ ID NO: 31, SEQ ID NO: 39, SEQ ID NO: 44, SEQ ID NO: 48, SEQ ID NO: 52, SEQ ID NO: 55, SEQ ID NO: 57, SEQ ID NO: 60, or SEQ ID NO: 67.

In certain embodiments, the anti-CD3 antibody or an antigen-binding fragment thereof comprises a heavy chain variable region and a light chain variable region, wherein the heavy chain variable region and the light chain variable region are selected from the group consisting of:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 15, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 16;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 23;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 30, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 31;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 38, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 39;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 44;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 47, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 48;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 51, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 52;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 54, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 55;
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 56, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 57;
(k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 59, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 60; and
(l) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 66, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 67.

In certain embodiments, the anti-CD3 antibody or an antigen-binding fragment thereof comprises a heavy chain variable region comprising the amino acid sequence set forth in SEQ ID NO: 7, SEQ ID NO: 15, SEQ ID NO: 22, SEQ ID NO: 30, SEQ ID NO: 38, SEQ ID NO: 43, SEQ ID NO: 47, SEQ ID NO: 51, SEQ ID NO: 54, SEQ ID NO: 56, SEQ ID NO: 59, SEQ ID NO: 66, or SEQ ID NO: 70.

In certain embodiments, the anti-CD3 antibody or an antigen-binding fragment thereof comprises a light chain variable region comprising the amino acid sequence set forth in SEQ ID NO: 8, SEQ ID NO: 16, SEQ ID NO: 23, SEQ ID NO: 31, SEQ ID NO: 39, SEQ ID NO: 44, SEQ ID NO: 48, SEQ ID NO: 52, SEQ ID NO: 55, SEQ ID NO: 57, SEQ ID NO: 60, or SEQ ID NO: 67.

In certain embodiments, the anti-CD3 antibody or an antigen-binding fragment thereof comprises:
(a) a heavy chain variable region comprising the amino acid sequence set forth in SEQ ID NO: 7, SEQ ID NO: 15, SEQ ID NO: 22, SEQ ID NO: 30, SEQ ID NO: 38, SEQ ID NO: 43, SEQ ID NO: 47, SEQ ID NO: 51, SEQ ID NO: 54, SEQ ID NO: 56, SEQ ID NO: 59, or SEQ ID NO: 66; and
(b) a light chain variable region comprising the amino acid sequence set forth in SEQ ID NO: 8, SEQ ID NO: 16, SEQ ID NO: 23, SEQ ID NO: 31, SEQ ID NO: 39, SEQ ID NO: 44, SEQ ID NO: 48, SEQ ID NO: 52, SEQ ID NO: 55, SEQ ID NO: 57, SEQ ID NO: 60, or SEQ ID NO: 67.

In certain embodiments, the anti-CD3 antibody or an antigen-binding fragment thereof comprises:
(a) the heavy chain variable region comprises the amino acid sequence set forth in SEQ ID NO: 7, and the light chain variable region comprises the amino acid sequence set forth in SEQ ID NO: 8;
(b) the heavy chain variable region comprises the amino acid sequence set forth in SEQ ID NO: 15, and the light chain variable region comprises the amino acid sequence set forth in SEQ ID NO: 16;
(c) the heavy chain variable region comprises the amino acid sequence set forth in SEQ ID NO: 22, and the light chain variable region comprises the amino acid sequence set forth in SEQ ID NO: 23;
(d) the heavy chain variable region comprises the amino acid sequence set forth in SEQ ID NO: 30, and the light chain variable region comprises the amino acid sequence set forth in SEQ ID NO: 31;
(e) the heavy chain variable region comprises the amino acid sequence set forth in SEQ ID NO: 38, and the light chain variable region comprises the amino acid sequence set forth in SEQ ID NO: 39;
(f) the heavy chain variable region comprises the amino acid sequence set forth in SEQ ID NO: 43, and the light chain variable region comprises the amino acid sequence set forth in SEQ ID NO: 44;
(g) the heavy chain variable region comprises the amino acid sequence set forth in SEQ ID NO: 47, and the light chain variable region comprises the amino acid sequence set forth in SEQ ID NO: 48;
(h) the heavy chain variable region comprises the amino acid sequence set forth in SEQ ID NO: 51, and the light chain variable region comprises the amino acid sequence set forth in SEQ ID NO: 52;
(i) the heavy chain variable region comprises the amino acid sequence set forth in SEQ ID NO: 54, and the light chain variable region comprises the amino acid sequence set forth in SEQ ID NO: 55;
(j) the heavy chain variable region comprises the amino acid sequence set forth in SEQ ID NO: 56, and the light chain variable region comprises the amino acid sequence set forth in SEQ ID NO: 57;
(k) the heavy chain variable region comprises the amino acid sequence set forth in SEQ ID NO: 59, and the light chain variable region comprises the amino acid sequence set forth in SEQ ID NO: 60; or
(l) the heavy chain variable region comprises the amino acid sequence set forth in SEQ ID NO: 66, and the light chain variable region comprises the amino acid sequence set forth in SEQ ID NO: 67.

In certain embodiments, one or more of the CDR sequences have up to about 5 amino acid substitutions. In certain embodiments, one or more of the CDR sequences have up to about 3 amino acid substitutions. In certain embodiments, the antibody comprises a comprises a heavy chain constant region and/or a light chain constant region. In certain embodiments,
(a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 82, SEQ ID NO: 83, SEQ ID NO: 84, or SEQ ID NO: 85; and/or
(b) the light chain constant region comprises an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 86.

In certain embodiments,
(a) the heavy chain constant region comprises the amino acid sequence set forth in SEQ ID NO: 82, SEQ ID NO: 83, SEQ ID NO: 84, or SEQ ID NO: 85; and/or
(b) the light chain constant region comprises the amino acid sequence set forth in SEQ ID NO: 86.

In certain embodiments, the antibody comprises a human variable region framework region. In certain embodiments, the anti-CD3 antibody or antigen-binding fragment thereof is a fully human or an antigen-binding fragment thereof. In certain embodiments, the anti-CD3 antibody or antigen-binding fragment thereof is a chimeric antibody or an antigen-binding fragment thereof. In certain embodiments, the anti-CD3 antibody or antigen-binding fragment thereof is a humanized antibody or an antigen-binding fragment thereof.

In certain embodiments, the antigen-binding fragment is a Fab, Fab′, F(ab′)2, variable fragment (Fv), or single chain variable region (scFv). In certain embodiments, the antigen-binding fragment is an scFv.

In certain non-limiting embodiments, the presently disclosed subject matter provides an antibody or an antigen-binding fragment thereof, which cross-competes for binding to CD3 with an anti-CD3 antibody or an antigen-binding fragment thereof disclosed herein. In certain non-limiting embodiments, the presently disclosed subject matter provides an antibody or an antigen-binding fragment thereof, which binds to the same epitope region on CD3 with an anti-CD3 antibody or an antigen-binding fragment thereof disclosed herein.

In certain non-limiting embodiments, the presently disclosed subject matter provides a composition comprising the anti-CD3 antibody or antigen-binding fragment thereof disclosed herein. In certain embodiments, the composition is a pharmaceutical composition that further comprises a pharmaceutically acceptable carrier.

In certain non-limiting embodiments, the presently disclosed subject matter provides an immunoconjugate comprising the anti-CD3 antibody or antigen-binding fragment thereof disclosed herein, linked to a therapeutic agent. In certain embodiments, the therapeutic agent is a drug, a cytotoxin, or a radioactive isotope.

In certain non-limiting embodiments, the presently disclosed subject matter provides a composition comprising the immunoconjugate disclosed herein. In certain embodiments, the composition is a pharmaceutical composition that further comprises a pharmaceutically acceptable carrier.

In certain non-limiting embodiments, the presently disclosed subject matter provides a multi-specific molecule comprising the antibody or antigen-binding fragment thereof disclosed herein, linked to one or more functional moieties. In certain embodiments, the one or more functional moieties have a different binding specificity than the antibody or antigen binding fragment thereof.

In certain non-limiting embodiments, the presently disclosed subject matter provides a multi-specific molecule comprising a first binding specificity for CD3 and a second binding specificity. In certain embodiments, the first binding specificity comprises the antibody or antigen-binding fragment thereof of disclosed herein.

In certain embodiments, the second binding specificity comprises an anti-CD33 antibody or antigen-binding fragment thereof. In certain embodiments, the second binding specificity comprises a VH comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a VL comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92. In certain embodiments, the second binding specificity comprises a VH comprising the amino acid sequence set forth in SEQ ID NO: 93, and a VL comprising the amino acid sequence set forth in SEQ ID NO: 94.

In certain embodiments, the second binding specificity comprises an anti-U5 snRNP200 antibody or antigen-binding fragment thereof. In certain embodiments, the second binding specificity comprises a VH comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a VL comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the second binding specificity comprises a VH comprising the amino acid sequence set forth in SEQ ID NO: 122, and a VL comprising the amino acid sequence set forth in SEQ ID NO: 123. In certain embodiments, the multi-specific molecule comprises the amino acid sequence set forth in SEQ ID NO: 128 or SEQ ID NO: 130.

In certain embodiments, the second binding specificity comprises an anti-CD371 antibody or antigen-binding fragment thereof. In certain embodiments, the second binding specificity comprises a VH comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134; and a VL comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137. In certain embodiments, the second binding specificity comprises a VH comprising the amino acid sequence set forth in SEQ ID NO: 138, and a VL comprising the amino acid sequence set forth in SEQ ID NO: 139. In certain embodiments, the multi-specific molecule comprises the amino acid sequence set forth in SEQ ID NO: 144.

In certain non-limiting embodiments, the presently disclosed subject matter provides a composition comprising the multi-specific molecule disclosed herein. In certain embodiments, the composition is a pharmaceutical composition that further comprises a pharmaceutically acceptable carrier.

In certain non-limiting embodiments, the presently disclosed subject matter provides a an immunocytokine comprising an antibody or antigen-binding fragment thereof disclosed herein and an IL-18 polypeptide or a fragment thereof. In certain embodiments, the IL-18 polypeptide comprises the amino acid sequence set forth in SEQ ID NO: 71.

In certain non-limiting embodiments, the presently disclosed subject matter provides an immunocytokine comprising a multi-specific molecule disclosed herein and an IL-18 polypeptide or a fragment thereof. In certain embodiments, the IL-18 polypeptide comprises the amino acid sequence set forth in SEQ ID NO: 71.

In certain non-limiting embodiments, the presently disclosed subject matter provides a composition comprising the immunocytokine disclosed herein. In certain embodiments, the composition is a pharmaceutical composition that further comprises a pharmaceutically acceptable carrier.

In certain non-limiting embodiments, the presently disclosed subject matter provides a nucleic acid that encodes an antibody or antigen-binding fragment thereof, a multi-specific molecule, or an immunocytokine disclosed herein. In certain non-limiting embodiments, the presently disclosed subject matter provides a vector comprising a nucleic acid disclosed herein. In certain non-limiting embodiments, the presently disclosed subject matter provides a host cell comprising a vector disclosed herein.

In certain non-limiting embodiments, the presently disclosed subject matter provides an immunoresponsive cell comprising the antibody or antigen-binding fragment thereof, the immunoconjugate, the multi-specific molecule, the immunocytokine, the nucleic acid, or the vector disclosed herein. In certain embodiments, the cell further comprises an antigen-recognizing receptor. In certain embodiments, the antigen-recognizing receptor is a recombinant T cell receptor (TCR), a chimeric antigen receptor (CAR), or a TCR like fusion molecule. In certain embodiments, the antigen-recognizing receptor is a CAR. In certain embodiments, the antigen is a tumor antigen. In certain embodiments, the tumor antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD371, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGFIR, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PRI), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, U5 snRNP200, and ZDHHC11. In certain embodiments, the immunoresponsive cell is a cell of the lymphoid lineage or a cell of the myeloid lineage. In certain embodiments, the cell is selected from the group consisting of a T cell, a Natural Killer (NK) cell, a B cell, a monocyte, and a macrophage, a pluripotent stem cell from which a lymphoid cell may be differentiated, a pluripotent stem cell from which a myeloid cell may be differentiated, and combinations thereof. In certain embodiments, the cell is a T cell. In certain embodiments, the cell is a Natural Killer (NK) cell. In certain embodiments, the cell is autologous. In certain embodiments, the cell is allogeneic.

In certain non-limiting embodiments, the presently disclosed subject matter provides a composition comprising the immunoresponsive cell disclosed herein. In certain embodiments, the composition is a pharmaceutical composition further comprising a pharmaceutically acceptable excipient.

In certain non-limiting embodiments, the presently disclosed subject matter provides a method for detecting CD3 in a whole cell, a tissue, or a blood sample, comprising:
(a) contacting a cell, tissue or blood sample with the antibody or antigen-binding fragment thereof disclosed herein, wherein the antibody or antigen-binding fragment thereof comprises a detectable label; and
(b) determining the amount of the labeled antibody or antigen-binding fragment thereof bound to the cell, tissue or blood sample by measuring the amount of detectable label associated with said cell or tissue, wherein the amount of bound antibody or antigen-binding fragment thereof indicates the amount of CD3 in the cell, tissue or blood sample.

In certain non-limiting embodiments, the presently disclosed subject matter provides a method of treating or ameliorating a disease or disorder in a subject, reducing tumor burden in a subject, treating and/or preventing a tumor in a subject, and/or increasing or lengthening survival of a subject having a tumor, the method comprising administering to the subject the antibody or antigen-binding fragment, the immunoconjugate, the multi-specific molecule, the immunocytokine, the cell, or the composition disclosed herein. In certain embodiments, the disease or disorder is a tumor. In certain embodiments, the method reduces the number of the tumor cells, reduces the tumor size, and/or eradicates the tumor in the subject. In certain embodiments, the method reduces or eradicates tumor burden in the subject. In certain embodiments, the tumor is cancer. In certain embodiments, the tumor is hematological cancer or solid tissue cancer. In certain embodiments, the hematological cancer is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms. In certain embodiments, the hematological cancer is acute myeloid leukemia (AML).

In certain non-limiting embodiments, the presently disclosed subject matter provides a method of inducing myeloid depletion in a subject in need thereof, comprising administering to the subject the multi-specific molecule or the composition disclosed herein.

In certain embodiments, the subject is a human.

In certain non-limiting embodiments, the presently disclosed subject matter provides a kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof, the immunoconjugate, the multi-specific molecule, the immunocytokine, the cell, or the composition disclosed herein.

In certain embodiments, the kit further comprises written instructions for using the antibody or antigen-binding fragment thereof, immunoconjugate, multi-specific molecule, or composition for treating or ameliorating a disease or disorder in a subject, treating or ameliorating a disease or disorder in a subject, reducing tumor burden in a subject, treating and/or preventing a tumor in a subject, and/or increasing or lengthening survival of a subject having a tumor.

### BRIEF DESCRIPTION OF THE FIGURES

The following Detailed Description, given by way of example, but not intended to limit the invention to specific embodiments described, may be understood in conjunction with the accompanying drawings.

FIG. 1 depicts a schematic of the strategy used to generate, screen, clone, and validate a panel of CD3& antibodies.

FIGS. 2A-2C illustrate the Quality Control (QC) analysis of the immunogens and antibodies.

FIGS. 3A-3C illustrate the polyclonal phage screening approach.

FIG. 4 depicts the EC50 ranking of the presently disclosed antibodies as assessed by FACS analysis using Jurkat cells.

FIGS. 5A and 5B illustrate the results of competition assays with UCHT1.

FIGS. 6A and 6B depict the functional EC50 Ranking of the presently disclosed antibodies as assessed by NFAT assay using Jurkat cells.

FIGS. 7A and 7B depict the Th1 functional EC50 ranking of the presently disclosed antibodies.

FIG. 8 illustrates the effects of the presently disclosed antibodies on Th1 proliferation.

FIGS. 9A and 9B show the functional EC50 ranking of the presently disclosed antibodies using purified T cells.

FIGS. 10A and 10B depict the functional EC50 ranking of the presently disclosed antibodies in mononuclear cells.

FIG. 11 shows sensorgram traces of the presently disclosed antibodies.

FIG. 12 shows the “Off Rate” analysis derived from the sensorgram of FIG. 11 and its relationship with the EC50 values.

FIG. 13 shows EC50 values and Th1 proliferation values of the presently disclosed antibodies.

FIG. 14 shows a table summarizing the binding affinity and functional data for the presently disclosed antibodies.

FIGS. 15A-15E depict sequence liability analysis. FIG. 15A shows heavy chain CDR3 percent similarity (SEQ ID NOs: 26, 41, 11, 46, 19, 34 and 128, respectively top to bottom first appearance). FIG. 15B shows heavy chain CDR3 percent similarity (length=8) (SEQ ID NO: 26).

FIG. 15C shows heavy chain CDR1, CDR2, and full-length percent similarity matrices. FIG. 15D shows heavy chain CDR3 percent similarity (length=11) (SEQ ID NOs: 46 and 19, respectively top to bottom first appearance). FIG. 15E shows heavy chain CDR1, CDR2, and full-length percent similarity matrices (length=11).

FIGS. 16A and 16B show the in-silico analysis of the presently disclosed antibodies. FIG. 16A shows in silico analysis of heavy chain CDRs (SEQ ID NOs: 32, 1, 24, 68, 9, 18, 40, 33, 2, 25, 69, 10, 19, 41, 34, 3, 26, 70, 11, 20, and 42, respectively top to bottom, left to right, first appearance). FIG. 16B shows in silico analysis of light chain CDRs (SEQ ID NOs: 35, 4, 27, 71, 12, 21, 43, 36, 5, 28, 72, 13, 22, 44, 37, 6, 29, 73, 14, 23, and 45, respectively top to bottom, left to right, first appearance).

FIGS. 17A-17C depict the functional characterization of the presently disclosed antibodies. FIG. 17A is a schematic representing the scFv-Fc fusion bivalent format. FIG. 17B shows Th1 proliferation of the presently disclosed antibodies having an scFv-Fc fusion bivalent format. FIG. 17C shows the binding efficiency of the reformatted binders by FACS EC50 analysis using Jurkat cells.

FIG. 18 is a schematic of various T-cell dependent bi-specifics formatted with Fc regions (TDBs).

FIGS. 19A and 19B depict the characterization of different multi-specific molecules disclosed herein. FIG. 19A shows the binding efficiency of the multispecific molecules disclosed herein by FACS analysis using Jurkat cells. FIG. 19B shows the binding efficiency of the multispecific molecules disclosed herein by FACS analysis using U937 cells.

FIGS. 20A and 20B depict the characterization of different multispecific molecules disclosed herein. FIG. 20A shows Th1 proliferation induced by the multispecific molecules disclosed herein. FIG. 20B shows the binding efficiency of the multispecific molecules disclosed herein relative to L2K.

FIG. 21 shows a schematic of the CD33 Knob/Hole Bispecific antibodies including the 3P14 anti-CD33 antibody or antigen-binding fragment thereof and the presently disclosed antibodies.

FIG. 22 shows an illustration of a method to assess the efficacy of multispecific molecules in vitro.

FIGS. 23A-23H show tumor killing and T cell proliferation induced by the presently disclosed multispecific molecules.

FIG. 24 shows the survival curves of AML xenograft mice receiving the presently disclosed multispecific molecules.

FIG. 25 shows various configurations of the multispecific molecules disclosed herein.

FIGS. 26A and 26B depict schematics of the presently disclosed immunocytokines. FIG. 26A shows a representation of a multispecific antibody including a cytokine. FIG. 26B shows a schematic illustrating an alternative multispecific molecule including a cytokine.

FIGS. 27A-27C depict exemplary multispecific antibodies encompassed by the presently disclosed subject matter. FIG. 27A shows the construct design of a bispecific antibody including a first binding specificity for U5 snRNP200 and a second binding specificity for CD3. FIG. 27B shows exemplary mechanisms of the bispecific antibodies depicted in FIG. 27A. FIG. 27C shows exemplary H- and L-constructs of the bispecific antibodies depicted in FIG. 27A.

FIGS. 28A-28J depict anti-tumor effects of T cells secreting bispecific antibodies depicted in FIG. 27A. FIG. 28A shows tumor killing effects of T cells from donor J3 on U937 AML cells. FIG. 28B shows tumor killing effects of T cells from donor J3 on OCI-AML3 AML cells. FIG. 28C shows tumor killing effects of T cells from donor J4 on U937 AML cells. FIG. 28D shows tumor killing effects of T cells from donor J4 on OCI-AML3 AML cells. FIG. 28E shows tumor killing effects of T cells from donor K3 on U937 AML cells. FIG. 28F shows tumor killing effects of T cells from donor K3 on OCI-AML2 AML cells. FIG. 28G shows tumor killing effects of T cells from donor K3 on OCI-AML3 AML cells. FIG. 28H shows tumor killing effects of T cells from donor K4 on U937 AML cells. FIG. 28I shows tumor killing effects of T cells from donor K4 on OCI-AML2 AML cells. FIG. 28J shows tumor killing effects of T cells from donor K4 on OCI-AML3 AML cells.

FIG. 29 shows tumor killing effects of T cells secreting a bispecific antibody including a first binding specificity for CD371 and a second binding specificity for CD3.

### DETAILED DESCRIPTION

The presently disclosed subject matter provides anti-CD3 antibodies. Non-limiting embodiments of the present disclosure are described by the present specification and Examples.

For purposes of clarity of disclosure and not by way of limitation, the detailed description is divided into the following subsections:
1. Definitions;
2, CD3;
3. Anti-CD3 Antibodies;
4. Cells;
5. Nucleic Acids encoding the Antibodies or Antigen-binding Fragments
6. Pharmaceutical Compositions and Methods of Treatment;
7. Diagnostic and Prognostic Methods;
8. Kits; and
9. Exemplary Embodiments.

### 1. Definitions

In the description that follows, certain conventions will be followed as regards the usage of terminology. Generally, terms used herein are intended to be interpreted consistently with the meaning of those terms as they are known to those of skill in the art.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding fragment” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies includes one or more fragments of an antibody that retain the ability to specifically bind to an antigen (e.g., a CD3 polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., Nature 1989; 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to CD3” is intended to refer to an antibody that binds to CD3ε (e.g., human CD3ε) with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less.

An “antibody that competes for binding” or “antibody that cross-competes for binding” with a reference antibody for binding to an antigen, e.g., CD3, refers to an antibody that blocks binding of the reference antibody to the antigen (e.g., CD3ε) in a competition assay by 50% or more, and conversely, the reference antibody blocks binding of the antibody to the antigen (e.g., CD3ε) in a competition assay by 50% or more. An exemplary competition assay is described in “Antibodies,” Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term” an antibody which binds specifically to an antigen (e.g., a CD3 polypeptide).”

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}.) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain.

Non-limiting examples of linkers, e.g., for use in generating an scFv, are disclosed in Shen et al., Anal Chem (2008); 80 (6): 1910-1917 and WO 2014/087010, the contents of which are hereby incorporated by reference in their entireties. In certain embodiments, the linker is a G4S linker. In certain embodiments, the linker comprises or consists of the amino acid sequence set forth in SEQ ID NO: 75, which is provided below:

[SEQ ID NO: 75]
GGGGSGGGGSGGGSGGGGS

In certain embodiments, the linker comprises or consists of the amino acid sequence set forth in SEQ ID NO: 76, which is provided below:

[SEQ ID NO: 76]
GGGGSGGGGSGGGGS

In certain embodiments, the linker comprises or consists of the amino acid sequence set forth in SEQ ID NO: 77, which is provided below:

[SEQ ID NO: 77]
GGGGSGGGGSGGGGSGGGSGGGGS

In certain embodiments, the linker comprises or consists of the amino acid sequence set forth in SEQ ID NO: 78, which is provided below:

[SEQ ID NO: 78]
GGGGSGGGGSGGGGSGGGGSGGGSGGGGS

In certain embodiments, the linker comprises or consists of the amino acid sequence set forth in SEQ ID NO: 79, which is provided below:

[SEQ ID NO: 79]
GGGGS

In certain embodiments, the linker comprises or consists of the amino acid sequence set forth in SEQ ID NO: 80, which is provided below:

[SEQ ID NO: 80]
GGGGSGGGGS

In certain embodiments, the linker comprises or consists of the amino acid sequence set forth in SEQ ID NO: 80, which is provided below:

[SEQ ID NO: 80]
GGGGSGGGGS

In certain embodiments, the linker comprises or consists of the amino acid sequence set forth in SEQ ID NO: 115, which is provided below:

[SEQ ID NO: 115]
AAAGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 1988; 85:5879-5883). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008; 27 (6): 455-51; Peter et al., J Cachexia Sarcopenia Muscle 2012 Aug. 12; Shich et al., J Imunol 2009; 183 (4): 2277-85; Giomarelli et al., Thromb Haemost 2007; 97 (6): 955-63; Fife eta., J Clin Invst 2006; 116 (8): 2252-61; Brocks et al., Immunotechnology 1997; 3 (3): 173-84; Moosmayer et al., Ther Immunol 1995; 2 (10:31-40). Agonistic scFvs having stimulatory activity have been described (see, e.g., Peter et al., J Bio. Chem 2003; 25278 (38): 36740-7; Xie et al., Nat Biotech 1997; 15(8): 768-71; Ledbetter et al., Crit Rev Immunol 1997; 17 (5-6): 427-55; Ho et al., BioChim Biophys Acta 2003; 1638 (3): 257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

“CDRs” are defined as the complementarity determining region amino acid sequences of an antibody which are the hypervariable regions of immunoglobulin heavy and light chains. Sec, e. g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at https://www.imgt.org/IMGTScientificChart/Nomenclature/IMGT-FRCDRdefinition.html.

The terms “isolated” denotes a degree of separation from original source or surroundings.

An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr (2007); B 848:79-87.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” (including references to a specific antibody, e.g. an anti-CD3 antibody) refers to one or more nucleic acid molecules encoding antibody heavy and light chains (or fragments thereof), including such nucleic acid molecule(s) in a single vector separate vectors, and such nucleic acid molecule(s) present at one or more locations in a host cell.

By “isolated cell” is meant a cell that is separated from the molecular and/or cellular components that naturally accompany the cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including, but not limited to, a cytotoxic agent.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition, and the form and effective concentration of the cells administered.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters; guinea pigs; rabbits; dogs; cats; sheep; pigs; goats; cattle; horses; and non-human primates such as apes and monkeys.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a tumor, e.g., a tumor associated with CD3.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when a CAR or a TCR like fusion molecule binds to an antigen, a formation of an immunological synapse occurs that includes clustering of many molecules near the bound receptor (e.g., CD4 or CD8, CD3g/d/e/z, etc.). This clustering of membrane bound signaling molecules allows for ITAM motifs contained within the CD3 chains to become phosphorylated. This phosphorylation in turn initiates a T cell activation pathway ultimately activating transcription factors, such as NF-kB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen (e.g., a tumor antigen). In certain embodiments, the antigen-recognizing receptor is capable of activating an immune or immunoresponsive cell (e.g., a T cell) upon its binding to the target antigen.

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain.

In certain embodiments, the extracellular antigen-binding domain of a CAR comprises a scFv. The scFv can be derived from fusing the variable heavy and light regions of an antibody. In certain embodiments, the scFv may be derived from Fab's (instead of from an antibody, e.g., obtained from Fab libraries). In certain embodiments, the scFv is fused to the transmembrane domain and then to the intracellular signaling domain.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

As used herein, the term “immunosuppressive activity” is meant induction of signal transduction or changes in protein expression in a cell (e.g., an activated immunoresponsive cell) resulting in a decrease in an immune response. Non-limiting examples of polypeptides suppressing or decreasing an immune response via their binding include CD47, PD-1, CTLA-4, BTLA, LAG-3, 2B4, and their corresponding ligands (including, but not limited to, SIRPa, PD-L1, PD-L2, TNFRSF14, CD48, and FGL-1). Such polypeptides are present in the tumor microenvironment and inhibit immune responses to neoplastic cells. In certain embodiments, inhibiting, blocking, or antagonizing the interaction of immunosuppressive polypeptides and/or their ligands enhances the immune response of the immunoresponsive cell.

As used herein, the term “immunostimulatory activity” is meant induction of signal transduction or changes in protein expression in a cell (e.g., an activated immunoresponsive cell) resulting in an increase in an immune response. Immunostimulatory activity may include pro-inflammatory activity. Non-limiting examples of polypeptides stimulating or increasing an immune response via their binding include CD28, CD40, OX-40, 4-1BB, GITR, and their corresponding ligands, including B7-1, B7-2, CD40L, OX-40L, 4-1BBL, GITRL. Such polypeptides are present in the tumor microenvironment and activate immune responses to neoplastic cells. In certain embodiments, promoting, stimulating, or agonizing pro-inflammatory polypeptides and/or their ligands enhances the immune response of the immunoresponsive cell.

The term “antigen-binding domain” as used herein refers to a domain capable of specifically binding a particular antigenic determinant or set of antigenic determinants present on a cell.

By “neoplasm” or “neoplasia” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia includes cancers, such as sarcomas, carcinomas, or plasmacytomas (malignant tumor of the plasma cells). The neoplasia can a primary tumor or primary cancer. In addition, the neoplasm can be in metastatic status.

By “receptor” is meant a polypeptide, or portion thereof, present on a cell membrane that selectively binds one or more ligand.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. In certain embodiments, the signal peptide is covalently joined to the 5′ terminus of the extracellular antigen-binding domain. In certain embodiments, the signal peptide comprises a CD8 polypeptide, e.g., the CAR comprises a truncated CD8 signal peptide. In certain embodiments, the truncated CD8 signal peptide comprises or consists of the amino acid sequence set forth in SEQ ID NO: 113. An exemplary nucleotide sequence encoding the truncated CD8 signal peptide of SEQ ID NO: 113 is set forth in SEQ ID NO: 114. SEQ ID NOs: 139 and 140 are provided below.

[SEQ ID NO: 113]
MALPVTALLLPLALLLHAARP
[SEQ ID NO: 114]
ATGGCGTTGCCGGTGACCGCGCTTTTGCTCCCTCTGGCCCTGCTCTTGC
ACGCCGCACGACCG

The terms “comprises”, “comprising”, and are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

Other aspects of the presently disclosed subject matter are described in the following disclosure and are within the ambit of the presently disclosed subject matter.

### 2. CD3

CD3 is a protein complex involved in the activation of T cell receptor signaling. CD3 protein complex includes a CD3γ polypeptide, a CD3δ polypeptide, two CD3ε polypeptides, and two CD3ζ polypeptides. The intracellular domain of each of CD3γ, CD3δ, and CD3ε polypeptides includes a single conserved motif known as an immunoreceptor tyrosine-based activation motif (ITAM), while the intracellular domain of the CD3ζ polypeptide includes three ITAM motifs.

CD3ε, also known as T-cell surface glycoprotein CD3 epsilon chain), is composed of an extracellular domain, a transmembrane domain, and a cytoplasmatic domain. The extracellular domain can bind proteins such as TOP2B, CD3EAP, and NCK2 (Nakano et al., Journal of Biological Chemistry 271.11 (1996): 6483-6489; Yamazaki et al., Journal of Biological Chemistry 274.26 (1999): 18173-18180; and Gil et al., Cell 109.7 (2002): 901-912).

In certain embodiments, the presently disclosed anti-CD3 antibodies or antigen-binding fragments thereof bind to human CD38. In certain embodiments, the human CD3ε comprises or consists of the amino acid sequence with a UniProt Reference No: P07766 (SEQ ID NO: 81) or a fragment thereof. SEQ ID NO: 81 is provided below. In certain embodiments, the human CD3E comprises an extracellular domain, a transmembrane domain, and a cytoplasmic domain. In certain embodiments, the extracellular domain comprises or consists of amino acids 26 to 126 of SEQ ID NO: 81. In certain embodiments, the transmembrane domain comprises or consists of amino acids 127 to 152 of SEQ ID NO: 81. In certain embodiments, the cytoplasmic domain comprises or consists of amino acids 153 to 207 of SEQ ID NO: 81.

[SEQ ID NO: 81]
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI

In certain embodiments, the CD3ε comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 81 or a fragment thereof.

In certain embodiments, the presently disclosed anti-CD3 antibodies or antigen-binding fragments thereof bind to a portion of human CD38. In certain embodiments, the presently disclosed anti-CD3 antibodies or antigen-binding fragments thereof bind to the extracellular domain of CD38. In certain embodiments, the presently disclosed anti-CD3 antibodies or antigen-binding fragments thereof bind to amino acids 26 to 126 of SEQ ID NO: 81.

### 3. Anti-CD3 Antibodies

The antibodies of the presently disclosed subject matter are characterized by particular functional features or properties of the antibodies. For example, the antibodies bind specifically to CD3ε (e.g., bind to human CD3ε).

In certain embodiments, a presently disclosed antibody or antigen-binding fragment binds to a cell expressing CD3ε (e.g., a T cell) with a Half maximal effective concentration (EC₅₀) value of from about 0.5 nM to about 50 nM, from about 5 nM to about 50 nM, from about 10 nM to about 50 nM, from about 20 nM to about 50 nM, from about 30 nM to about 50 nM, from about 40 nM to about 50 nM, or greater than about 50 nM. In certain embodiments, a presently disclosed antibody or antigen-binding fragment binds to a cell expressing CD3ε (e.g., a T cell) with an EC₅₀ value from about 0.5 nM to about 5 nM. In certain embodiments, a presently disclosed antibody or antigen-binding fragment binds to a cell expressing CD3ε (e.g., a T cell) with an EC₅₀ value of about 0.59 nM. In certain embodiments, a presently disclosed antibody or antigen-binding fragment binds to a cell expressing CD3ε (e.g., a T cell) with an EC₅₀ value of about 1.3 nM. In certain embodiments, a presently disclosed antibody or antigen-binding fragment binds to a cell expressing CD3ε (e.g., a T cell) with an EC₅₀ value from about 20 nM to about 40 nM. In certain embodiments, a presently disclosed antibody or antigen-binding fragment binds to a cell expressing CD3ε (e.g., a T cell) with an EC₅₀ value of about 25 nM. In certain embodiments, a presently disclosed antibody or antigen-binding fragment binds to a cell expressing CD3ε (e.g., a T cell) with an EC₅₀ value of about 34 nM.

In certain embodiments, a presently disclosed antibody or antigen-binding fragment binds to a cell expressing CD3ε (e.g., a T cell) with an off-rate (K_{off}) value of from about 1×10⁻⁴ sec⁻¹ to about 5× 10⁻² sec⁻¹, from about 5×10⁻⁴ sec⁻¹ to about 5×10⁻² sec⁻¹, from about 1×10⁻³ sec⁻¹ to about 5×10⁻² sec⁻¹, from about 5×10⁻³ sec⁻¹ to about 5×10⁻² sec⁻¹, from about 1×10⁻² sec⁻¹ to about 5×10⁻² sec⁻¹, or less than about 5×10⁻² sec⁻¹. In certain embodiments, a presently disclosed antibody or antigen-binding fragment binds to a cell expressing CD3ε (e.g., a T cell) with a K_{off} value from about 1×10⁻³ sec⁻¹ to about 5×10⁻³ sec⁻¹. In certain embodiments, a presently disclosed antibody or antigen-binding fragment binds to a cell expressing CD3ε (e.g., a T cell) with a K_{off} value of about 3.24×10⁻³ sec⁻¹. In certain embodiments, a presently disclosed antibody or antigen-binding fragment binds to a cell expressing CD3ε (e.g., a T cell) with a K_{off} value of about 1.91×10⁻³ sec⁻¹. In certain embodiments, a presently disclosed antibody or antigen-binding fragment binds to a cell expressing CD3ε (e.g., a T cell) with a K_{off} value of about 1.98×10⁻³ sec⁻¹. In certain embodiments, a presently disclosed antibody or antigen-binding fragment binds to a cell expressing CD3ε (e.g., a T cell) with a K_{off} value from about 1×10⁻⁴ sec⁻¹ to about 5×10⁻⁴ sec⁻¹. In certain embodiments, a presently disclosed antibody or antigen-binding fragment binds to a cell expressing CD3ε (e.g., a T cell) with a K_{off} value of about 3.18×10⁻⁴ sec⁻¹.

The heavy and light chains of a presently disclosed antibody or antigen-binding fragment can be full-length (e.g., an antibody can include at least one (e.g., one or two) complete heavy chains, and at least one (e.g., one or two) complete light chains) or can include an antigen-binding fragment (a Fab, F(ab′)₂, Fv or a single chain Fv fragment (“scFv”)). In certain embodiments, the antibody heavy chain constant region is chosen from, e.g., IgG1, IgG2, IgG3, IgG4, IgM, IgA1, IgA2, IgD, and IgE. In certain embodiments, the antibody heavy chain constant region is chosen from, e.g., IgG1, IgG2, IgG3, and IgG4. In certain embodiments, the immunoglobulin isotype is IgG1 (e.g., human IgG1). The choice of antibody isotype can depend on the immune effector function that the antibody is designed to elicit. In certain embodiments, the antibody light chain constant region is chosen from, e.g., kappa or lambda. In certain embodiments, the antibody light chain constant region is kappa.

In constructing a recombinant immunoglobulin, appropriate amino acid sequences for constant regions of various immunoglobulin isotypes and methods for the production of a wide array of antibodies are known to those of skill in the art.

3.1. Single-Chain Variable Fragments (scFvs)

In certain embodiments, the presently disclosed subject matter includes antibodies or antigen-binding fragments thereof that have the scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes, etc. to the antibody for example, for use in antigen quantitation studies, to immobilize the antibody for affinity measurements, for targeted delivery of a therapeutic agent, to test for Fc-mediated cytotoxicity using immune effector cells and many other applications.

The results presented here highlight the specificity, sensitivity, and utility of the presently disclosed antibodies or antigen-binding fragments in targeting a CD3ε polypeptide (e.g., a human CD3& polypeptide).

In certain embodiments, the anti-CD3 antibody is an scFv, an scFv-Fc fusion protein or a full-length human IgG (or antibody fragment thereof) with V_{H} and V_{L} regions or CDRs selected from Table 1. In certain embodiments, the anti-CD3 scFv comprises a V_{H} comprising the amino acid sequence set forth in SEQ ID NO: 7. In certain embodiments, the anti-CD3 scFv comprises a V_{L} comprising the amino acid sequence set forth in SEQ ID NO: 8. SEQ ID NO: 7 and 8 are provided in Table 1. In certain embodiments, the scFv is designated as “01F05C01”.

In certain embodiments, the anti-CD3 scFv comprises a V_{H} comprising the amino acid sequence set forth in SEQ ID NO: 7 and a V_{L} comprising the amino acid sequence set forth in SEQ ID NO: 8.

In certain embodiments, the anti-CD3 scFv comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. SEQ ID NOs: 1-3 are provided in Table 1.

In certain embodiments, the anti-CD3 scFv comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. SEQ ID NOs: 4-6 are provided in Table 1.

In certain embodiments, the anti-CD3 scFv comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, a CDR3 comprising the amino acid sequence set forth in SEQ ID NO: 3 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification, and a CDR3 comprising the amino acid sequence set forth in SEQ ID NO: 6 or a conservative modification thereof.

In certain embodiments, the anti-CD3 scFv comprises a V_{H} comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the anti-CD3 scFv comprises a V_{H} comprising the amino acid sequence set forth in SEQ ID NO: 7, and a V_{L} comprising the amino acid sequence set forth in SEQ ID NO: 8. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 1
(01F05C01)

| CDRs | 1 | 2 | 3 |
|---|---|---|---|
| V_{H} | GYTFTAYY | INPNSGGT | TRGTPRYNWKSYNY |
| | [SEQ ID NO: 1] | [SEQ ID NO: 2] | GMDV |
| | | | [SEQ ID NO: 3] |
| V_{L} | QSLVHSDGNTY | RIS | MQATQFPIT |
| | [SEQ ID NO: 4] | [SEQ ID NO: 5] | [SEQ ID NO: 6] |

| | |
|---|---|
| Full V_{H} | QVQLVQSGAEVKKPGASVKVSCKASGYTFTAYYIHWVRQAPGQGLEW |
| | MGWINPNSGGTNYAQMFQGRVTMTRDTSISAAYMELSRLSSDDTAVYY |
| | CTRGTPRYNWKSYNYGMDVWGQGTTVTVSS [SEQ ID NO: 7] |
| Full V_{L} | DIVVTQTPLSSPVTLGQPASISCRSSQSLVHSDGNTYLSWLQQRPGQPPRL |
| | LIYRISNRFSGVPDRFSGSGAGTDFTLKISRVEAEDVGVYYCMQATQFPIT |
| | FGQGTRLEIK [SEQ ID NO: 8] |

In certain embodiments, the anti-CD3 antibody is an scFv, an scFv-Fc fusion protein or a full-length human IgG (or antibody fragment thereof) with V_{H} and V_{L} regions or CDRs selected from Table 2. In certain embodiments, the anti-CD3 scFv comprises a V_{H} comprising the amino acid sequence set forth in SEQ ID NO: 15. In certain embodiments, the anti-CD3 scFv comprises a V_{L} comprising the amino acid sequence set forth in SEQ ID NO: 16. In certain embodiments, the anti-CD3 scFv comprises a V_{H} comprising the amino acid sequence set forth in SEQ ID NO: 15 and a V_{L} comprising the amino acid sequence set forth in SEQ ID NO: 16. SEQ ID NO: 15 and 16 are provided in Table 2. In certain embodiments, the scFv is designated as “07B09”.

In certain embodiments, the anti-CD3 scFv comprises a V_{H} comprising a CDR1 comprising the amino acid sequence set forth in SEQ ID NO: 9 or a conservative modification thereof, a CDR2 comprising the amino acid sequence set forth in SEQ ID NO: 10 or a conservative modification thereof, and a CDR3 comprising the amino acid sequence set forth in SEQ ID NO: 11 or a conservative modification thereof. SEQ ID NOs: 9-11 are provided in Table 2.

In certain embodiments, the anti-CD3 scFv comprises a V_{L} comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof. SEQ ID NOs: 12-14 are provided in Table 2.

In certain embodiments, the anti-CD3 scFv comprises a V_{H} comprising a CDR1 comprising the amino acid sequence set forth in SEQ ID NO: 9 or a conservative modification thereof, a CDR2 comprising the amino acid sequence set forth in SEQ ID NO: 10 or a conservative modification thereof, a CDR3 comprising the amino acid sequence set forth in SEQ ID NO: 11 or a conservative modification thereof; and a V_{L} comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification.

In certain embodiments, the anti-CD3 scFv comprises a V_{H} comprising a CDR1 comprising the amino acid sequence set forth in SEQ ID NO: 9, a CDR2 comprising the amino acid sequence set forth in SEQ ID NO: 10, a CDR3 comprising the amino acid sequence set forth in SEQ ID NO: 11; and a V_{L} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14.

In certain embodiments, the anti-CD3 scFv comprises a V_{H} comprising the amino acid sequence set forth in SEQ ID NO: 15, and a V_{L} comprising the amino acid sequence set forth in SEQ ID NO: 16. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 2
(07B09)

| CDRs | 1 | 2 | 3 |
|---|---|---|---|
| V_{H} | GFTFSSYW [SEQ ID | MRKDGSEK [SEQ ID | ARERSTGYFDY [SEQ |
| | NO: 9] | NO: 10] | ID NO: 11] |
| V_{L} | QRISTW [SEQ ID NO: | KAS [SEQ ID NO: 13] | QQYISYTWT [SEQ ID |
| | 12] | | NO: 14] |

| | |
|---|---|
| Full V_{H} | EVQLVESGGGLVQPGGSLRLSCAASGFTFSSYWMTWVRQAPGKGLEWV |
| | ANMRKDGSEKYYVDSVKGRFTISRDNAKNSLYLHMNSLRAEDTAVYYC |
| | ARERSTGYFDYWGQGTLVIVSS [SEQ ID NO: 15] |
| Full V_{L} | DIQMTQSPSTLSASVGDRVTITCRASQRISTWLAWYQQKAGKAPKLLIY |
| | KASSLESGVPSRFSGSGSGTEFTLTIRSLEPDDFATYYCQQYISYTWTFGQ |
| | GTKVEIK [SEQ ID NO: 16] |

In certain embodiments, the anti-CD3 antibody is an scFv, an scFv-Fc fusion protein or a full-length human IgG (or antibody fragment thereof) with V_{H} and V_{L} regions or CDRs selected from Table 3. In certain embodiments, the anti-CD3 scFv comprises a V_{H} comprising the amino acid sequence set forth in SEQ ID NO: 22. In certain embodiments, the anti-CD3 scFv comprises a V_{L} comprising the amino acid sequence set forth in SEQ ID NO: 23. In certain embodiments, the anti-CD3 scFv comprises a V_{H} comprising the amino acid sequence set forth in SEQ ID NO: 22 and a V_{L} comprising the amino acid sequence set forth in SEQ ID NO: 23. SEQ ID NO: 22 and 23 are provided in Table 3. In certain embodiments, the scFv is designated as “12C08”.

In certain embodiments, the anti-CD3 scFv comprises a V_{H} comprising a CDR1 comprising the amino acid sequence set forth in SEQ ID NO: 17 or a conservative modification thereof, a CDR2 comprising the amino acid sequence set forth in SEQ ID NO: 18 or a conservative modification thereof, and a CDR3 comprising the amino acid sequence set forth in SEQ ID NO: 19 or a conservative modification thereof. SEQ ID NOs: 17-19 are provided in Table 3.

In certain embodiments, the anti-CD3 scFv comprises a V_{L} comprising a CDR1 comprising the amino acid sequence set forth in SEQ ID NO: 20 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof. SEQ ID NOs: 13, 20, and 21 are provided in Table 3.

In certain embodiments, the anti-CD3 scFv comprises a V_{H} comprising a CDR1 comprising the amino acid sequence set forth in SEQ ID NO: 17 or a conservative modification thereof, a CDR2 comprising the amino acid sequence set forth in SEQ ID NO: 18 or a conservative modification thereof, a CDR3 comprising the amino acid sequence set forth in SEQ ID NO: 19 or a conservative modification thereof; and a V_{L} comprising a CDR1 comprising the amino acid sequence set forth in SEQ ID NO: 20 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification.

In certain embodiments, the anti-CD3 scFv comprises a V_{H} comprising a CDR1 comprising the amino acid sequence set forth in SEQ ID NO: 17, a CDR2 comprising the amino acid sequence set forth in SEQ ID NO: 18, a CDR3 comprising the amino acid sequence set forth in SEQ ID NO: 19; and a V_{L} comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21.

In certain embodiments, the anti-CD3 scFv comprises a V_{H} comprising the amino acid sequence set forth in SEQ ID NO: 22, and a V_{L} comprising the amino acid sequence set forth in SEQ ID NO: 23. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 3
(12C08)

| CDRs | 1 | 2 | 3 |
|---|---|---|---|
| V_{H} | GFTFRSYW [SEQ ID | IKEDGSEK [SEQ ID | ARERREGYFDQ [SEQ |
| | NO: 17] | NO: 18] | ID NO: 19] |
| V_{L} | QSIGSW | KAS | QQYKSYSWT [SEQ ID |
| | [SEQ ID NO: 20] | [SEQ ID NO: 13] | NO: 21] |

| | |
|---|---|
| Full V_{H} | EEQLVESGGGLVQPGGSLRFSCEVSGFTFRSYWMSWVRQAPGKGLEWV |
| | ANIKEDGSEKYYVDSVKGRFTISRDNAKNSLYLQMNSLRAEDTAVYYC |
| | ARERREGYFDQWGQGTLVTVSS [SEQ ID NO: 22] |
| Full V_{L} | DIQMTQSPSTLSAFVGDRVTITCRASQSIGSWLAWYQQKPGKAPDLLIYK |
| | ASSLESGVPSRFSGSGSGTEFTLTISSLQPDDFATYYCQQYKSYSWTFGQ |
| | GTKVEIK [SEQ ID NO: 23] |

In certain embodiments, the anti-CD3 antibody is an scFv, an scFv-Fc fusion protein or a full-length human IgG (or antibody fragment thereof) with V_{H} and V_{L} regions or CDRs selected from Table 4. In certain embodiments, the anti-CD3 scFv comprises a V_{H} comprising the amino acid sequence set forth in SEQ ID NO: 30. In certain embodiments, the anti-CD3 scFv comprises a V_{L} comprising the amino acid sequence set forth in SEQ ID NO: 31. In certain embodiments, the anti-CD3 scFv comprises a V_{H} comprising the amino acid sequence set forth in SEQ ID NO: 30 and a V_{L} comprising the amino acid sequence set forth in SEQ ID NO: 31. SEQ ID NO: 30 and 31 are provided in Table 4. In certain embodiments, the scFv is designated as “13D02”.

In certain embodiments, the anti-CD3 scFv comprises a V_{H} comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof. SEQ ID NOs: 24-26 are provided in Table 4.

In certain embodiments, the anti-CD3 scFv comprises a V_{L} comprising a CDR1 comprising the amino acid sequence set forth in SEQ ID NO: 27 or a conservative modification thereof, a CDR2 comprising the amino acid sequence set forth in SEQ ID NO: 28 or a conservative modification thereof, and a CDR3 comprising the amino acid sequence set forth in SEQ ID NO: 29 or a conservative modification thereof. SEQ ID NOs: 27-29 are provided in Table 4.

In certain embodiments, the anti-CD3 scFv comprises a V_{H} comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 27 or a conservative modification thereof, a CDR2 comprising the amino acid sequence set forth in SEQ ID NO: 28 or a conservative modification thereof, and a CDR3 comprising the amino acid sequence set forth in SEQ ID NO: 29 or a conservative modification.

In certain embodiments, the anti-CD3 scFv comprises a V_{H} comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a V_{L} comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29.

In certain embodiments, the anti-CD3 scFv comprises a V_{H} comprising the amino acid sequence set forth in SEQ ID NO: 30, and a V_{L} comprising the amino acid sequence set forth in SEQ ID NO: 31. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 4
(13D02)

| CDRs | 1 | 2 | 3 |
|---|---|---|---|
| V_{H} | GFTFSRFN [SEQ ID | ISSRSSYI [SEQ ID NO: | AGGGYFDY [SEQ ID |
| | NO: 24] | 25] | NO: 26] |
| V_{L} | QSLLHSNGYNY [SEQ | LGS [SEQ ID NO: 28] | IQALQTPYT [SEQ ID |
| | ID NO: 27] | | NO: 29] |

| | |
|---|---|
| Full V_{H} | EVQLVESGGGLVKPGGSLRLSCAASGFTFSRFNMNWVRQTPGKGLEWV |
| | SSISSRSSYIYYADSVKGRFTISRDNAKKSLYLQMNSLRAEDTAVYYCAG |
| | GGYFDYWGQGTLVTVSS [SEQ ID NO: 30] |
| Full V_{L} | DIVMTQSPLSLPVTPGEPAAISCRSSQSLLHSNGYNYLDWYLQKPGQSPQ |
| | LLIYLGSNRASGVPDRFSGSGSGTDFTLKISRVEAEDVGVYYCIQALQTP |
| | YTFGQGTKLEIK [SEQ ID NO: 31] |

In certain embodiments, the anti-CD3 antibody is an scFv, an scFv-Fc fusion protein or a full-length human IgG (or antibody fragment thereof) with V_{H} and V_{L} regions or CDRs selected from Table 5. In certain embodiments, the anti-CD3 scFv comprises a V_{H} comprising the amino acid sequence set forth in SEQ ID NO: 38. In certain embodiments, the anti-CD3 scFv comprises a V_{L} comprising the amino acid sequence set forth in SEQ ID NO: 39. In certain embodiments, the anti-CD3 scFv comprises a V_{H} comprising the amino acid sequence set forth in SEQ ID NO: 38 and a V_{L} comprising the amino acid sequence set forth in SEQ ID NO: 39. SEQ ID NO: 38 and 39 are provided in Table 5. In certain embodiments, the scFv is designated as “01B08B01”.

In certain embodiments, the anti-CD3 scFv comprises a V_{H} comprising a CDR1 comprising the amino acid sequence set forth in SEQ ID NO: 32 or a conservative modification thereof, a CDR2 comprising the amino acid sequence set forth in SEQ ID NO: 33 or a conservative modification thereof, and a CDR3 comprising the amino acid sequence set forth in SEQ ID NO: 34 or a conservative modification thereof. SEQ ID NOs: 32-34 are provided in Table 5.

In certain embodiments, the anti-CD3 scFv comprises a V_{L} comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 37 or a conservative modification thereof. SEQ ID NOs: 35-37 are provided in Table 5.

In certain embodiments, the anti-CD3 scFv comprises a V_{H} comprising a CDR1 comprising the amino acid sequence set forth in SEQ ID NO: 32 or a conservative modification thereof, a CDR2 comprising the amino acid sequence set forth in SEQ ID NO: 33 or a conservative modification thereof, a CDR3 comprising the amino acid sequence set forth in SEQ ID NO: 34 or a conservative modification thereof; and a V_{L} comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 37 or a conservative modification.

In certain embodiments, the anti-CD3 scFv comprises a V_{H} comprising a CDR1 comprising the amino acid sequence set forth in SEQ ID NO: 32, a CDR2 comprising the amino acid sequence set forth in SEQ ID NO: 33, a CDR3 comprising the amino acid sequence set forth in SEQ ID NO: 34; and a V_{L} comprising a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 37.

In certain embodiments, the anti-CD3 scFv comprises a V_{H} comprising the amino acid sequence set forth in SEQ ID NO: 38, and a V_{L} comprising the amino acid sequence set forth in SEQ ID NO: 39. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 5
(01B08B01)

| CDRs | 1 | 2 | 3 |
|---|---|---|---|
| V_{H} | GYTFTGYY | INPYRSGT | ARGAGTTFYNAFDI |
| | [SEQ ID NO: 32] | [SEQ ID NO: 33] | [SEQ ID NO: 34] |
| V_{L} | QGINNY | AAS | LQHNRYPFT |
| | [SEQ ID NO: 35] | [SEQ ID NO: 36] | [SEQ ID NO: 37] |

| | |
|---|---|
| Full V_{H} | QVQLVQSGAEVKKPGASVKVSCKAAGYTFTGYYMHWVRQAPGQGLE |
| | WMGWINPYRSGTNFAQKFQDRVTMTRDTSISTAYMELSSLSSDDTAIYY |
| | CARGAGTTFYNAFDIWGQGTMVTVSS [SEQ ID NO: 38] |
| Full V_{L} | DIQMTQSPSAMSASVGDRVTITCRASQGINNYLAWFQQKPGKVPKRLIY |
| | AASSLQSGVPSRFSGSGSGTEFTLTISSLQPEDFATYYCLQHNRYPFTFGP |
| | GTKVDIK [SEQ ID NO: 39] |

In certain embodiments, the anti-CD3 antibody is an scFv, an scFv-Fc fusion protein or a full-length human IgG (or antibody fragment thereof) with V_{H} and V_{L} regions or CDRs selected from Table 6. In certain embodiments, the anti-CD3 scFv comprises a V_{H} comprising the amino acid sequence set forth in SEQ ID NO: 43. In certain embodiments, the anti-CD3 scFv comprises a V_{L} comprising the amino acid sequence set forth in SEQ ID NO: 44. In certain embodiments, the anti-CD3 scFv comprises a V_{H} comprising the amino acid sequence set forth in SEQ ID NO: 43 and a V_{L} comprising the amino acid sequence set forth in SEQ ID NO: 44. SEQ ID NO: 43 and 44 are provided in Table 6. In certain embodiments, the scFv is designated as “06G07A04”.

In certain embodiments, the anti-CD3 scFv comprises a V_{H} comprising a CDR1 comprising the amino acid sequence set forth in SEQ ID NO: 40 or a conservative modification thereof, a CDR2 comprising the amino acid sequence set forth in SEQ ID NO: 18 or a conservative modification thereof, and a CDR3 comprising the amino acid sequence set forth in SEQ ID NO: 41 or a conservative modification thereof. SEQ ID NOs: 18, 40, 41 are provided in Table 6.

In certain embodiments, the anti-CD3 scFv comprises a V_{L} comprising a CDR1 comprising the amino acid sequence set forth in SEQ ID NO: 42 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof. SEQ ID NOs: 13, 21, and 42 are provided in Table 6.

In certain embodiments, the anti-CD3 scFv comprises a V_{H} comprising a CDR1 comprising the amino acid sequence set forth in SEQ ID NO: 40 or a conservative modification thereof, a CDR2 comprising the amino acid sequence set forth in SEQ ID NO: 18 or a conservative modification thereof, a CDR3 comprising the amino acid sequence set forth in SEQ ID NO: 41 or a conservative modification thereof; and a V_{L} comprising a CDR1 comprising the amino acid sequence set forth in SEQ ID NO: 42 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification.

In certain embodiments, the anti-CD3 scFv comprises a V_{H} comprising a CDR1 comprising the amino acid sequence set forth in SEQ ID NO: 40, a CDR2 comprising the amino acid sequence set forth in SEQ ID NO: 18, a CDR3 comprising the amino acid sequence set forth in SEQ ID NO: 41; and a V_{L} comprising a CDR1 comprising the amino acid sequence set forth in SEQ ID NO: 42, a CDR2 comprising the amino acid sequence set forth in SEQ ID NO: 13, and a CDR3 comprising the amino acid sequence set forth in SEQ ID NO: 21.

In certain embodiments, the anti-CD3 scFv comprises a V_{H} comprising the amino acid sequence set forth in SEQ ID NO: 43, and a V_{L} comprising the amino acid sequence set forth in SEQ ID NO: 44. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 6
(06G07A04)

| CDRs | 1 | 2 | 3 |
|---|---|---|---|
| V_{H} | GFTFSSFW [SEQ ID | IKEDGSEK [SEQ ID | ARERRTGYFDY |
| | NO: 40] | NO: 18] | [SEQ ID NO: 41] |
| V_{L} | QSIGSW [SEQ ID NO: | KAS [SEQ ID NO: 13] | QQYKSYSWT [SEQ |
| | 42] | | ID NO: 21] |

| | |
|---|---|
| Full V_{H} | EVQLVESGGGLVQPGGSLRLSCAASGFTFSSFWMSWVRQAPGKGLE |
| | WVANIKEDGSEKYYVDSVKGRFTISRDNAKNSLFLQMNSLRAEDTAV |
| | YYCARERRTGYFDYWGQGTLVTVSS [SEQ ID NO: 43] |
| Full V_{L} | DIQMTQSSSTLSASVGDRVTITCRASQSIGSWLAWFQQKPGKAPKLLI |
| | YKASSLESGVPSRFSGSGSGTEFTLTISSLQPDDFAIYYCQQYKSYSWT |
| | FGQGTKVEIK [SEQ ID NO: 44] |

In certain embodiments, the anti-CD3 antibody is an scFv, an scFv-Fc fusion protein or a full-length human IgG (or antibody fragment thereof) with V_{H} and V_{L} regions or CDRs selected from Table 7. In certain embodiments, the anti-CD3 scFv comprises a V_{H} comprising the amino acid sequence set forth in SEQ ID NO: 47. In certain embodiments, the anti-CD3 scFv comprises a V_{L} comprising the amino acid sequence set forth in SEQ ID NO: 48. In certain embodiments, the anti-CD3 scFv comprises a V_{H} comprising the amino acid sequence set forth in SEQ ID NO: 47 and a V_{L} comprising the amino acid sequence set forth in SEQ ID NO: 48. SEQ ID NO: 47 and 48 are provided in Table 7. In certain embodiments, the scFv is designated as “08G12”.

In certain embodiments, the anti-CD3 scFv comprises a V_{H} comprising a CDR1 comprising the amino acid sequence set forth in SEQ ID NO: 45 or a conservative modification thereof, a CDR2 comprising the amino acid sequence set forth in SEQ ID NO: 18 or a conservative modification thereof, and a CDR3 comprising the amino acid sequence set forth in SEQ ID NO: 46 or a conservative modification thereof. SEQ ID NOs: 18, 45, and 46 are provided in Table 7.

In certain embodiments, the anti-CD3 scFv comprises a V_{L} comprising a CDR1 comprising the amino acid sequence set forth in SEQ ID NO: 20 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof. SEQ ID NOs: 13, 20, and 21 are provided in Table 7.

In certain embodiments, the anti-CD3 scFv comprises a V_{H} comprising a CDR1 comprising the amino acid sequence set forth in SEQ ID NO: 45 or a conservative modification thereof, a CDR2 comprising the amino acid sequence set forth in SEQ ID NO: 18 or a conservative modification thereof, a CDR3 comprising the amino acid sequence set forth in SEQ ID NO: 46 or a conservative modification thereof; and a V_{L} comprising a CDR1 comprising the amino acid sequence set forth in SEQ ID NO: 20 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification.

In certain embodiments, the anti-CD3 scFv comprises a V_{H} comprising a CDR1 comprising the amino acid sequence set forth in SEQ ID NO: 45, a CDR2 comprising the amino acid sequence set forth in SEQ ID NO: 18, a CDR3 comprising the amino acid sequence set forth in SEQ ID NO: 46; and a V_{L} comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21.

In certain embodiments, the anti-CD3 scFv comprises a V_{H} comprising the amino acid sequence set forth in SEQ ID NO: 47, and a V_{L} comprising the amino acid sequence set forth in SEQ ID NO: 48. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 7
(08G12)

| CDRs | 1 | 2 | 3 |
|---|---|---|---|
| V_{H} | GFTFSRFW [SEQ ID | IKEDGSEK [SEQ ID | ARERREGYFDY [SEQ |
| | NO: 45] | NO: 18] | ID NO: 46] |
| V_{L} | QSIGSW [SEQ ID NO: | KAS [SEQ ID NO: 13] | QQYKSYSWT [SEQ ID |
| | 20] | | NO: 21] |

| | |
|---|---|
| Full V_{H} | EVQLVESGGGLVQSGGSLRLSCAASGFTFSRFWMTWVRQAPGKGLEWV |
| | ANIKEDGSEKYYVDSVKGRFTISRDNTKNSLYLQMNSLRAEDTAVYYCA |
| | RERREGYFDYWGQGTLVTVSS [SEQ ID NO: 47] |
| Full V_{L} | DIQMTQSPSTLSASVGDRVTITCRASQSIGSWLAWYQQKPGKAPKLLIYK |
| | ASSLESGVPSRFSGSGSGTEFTLTISSLQPDDFATYYCQQYKSYSWTFGH |
| | GTKVEIK [SEQ ID NO: 48] |

In certain embodiments, the anti-CD3 antibody is an scFv, an scFv-Fc fusion protein or a full-length human IgG (or antibody fragment thereof) with V_{H} and V_{L} regions or CDRs selected from Table 8 or Table 9. In certain embodiments, the anti-CD3 scFv comprises a V_{H} comprising the amino acid sequence set forth in SEQ ID NO: 51. In certain embodiments, the anti-CD3 scFv comprises a V_{L} comprising the amino acid sequence set forth in SEQ ID NO: 52. In certain embodiments, the anti-CD3 scFv comprises a V_{H} comprising the amino acid sequence set forth in SEQ ID NO: 51 and a V_{L} comprising the amino acid sequence set forth in SEQ ID NO: 52. SEQ ID NO: 51 and 52 are provided in Table 8 and Table 9. In certain embodiments, the scFv is designated as “09B01” or “09C02”.

In certain embodiments, the anti-CD3 scFv comprises a V_{H} comprising a CDR1 comprising the amino acid sequence set forth in SEQ ID NO: 24 or a conservative modification thereof, a CDR2 comprising the amino acid sequence set forth in SEQ ID NO: 49 or a conservative modification thereof, and a CDR3 comprising the amino acid sequence set forth in SEQ ID NO: 26 or a conservative modification thereof. SEQ ID NOs: 24, 26, and 49 are provided in Table 8 and Table 9.

In certain embodiments, the anti-CD3 scFv comprises a V_{L} comprising a CDR1 comprising the amino acid sequence set forth in SEQ ID NO: 20 or a conservative modification thereof, a CDR2 comprising the amino acid sequence set forth in SEQ ID NO: 28 or a conservative modification thereof, and a CDR3 comprising the amino acid sequence set forth in SEQ ID NO: 50 or a conservative modification thereof. SEQ ID NOs: 20, 28, and 50 are provided in Table 8 and Table 9.

In certain embodiments, the anti-CD3 scFv comprises a V_{H} comprising a CDR1 comprising the amino acid sequence set forth in SEQ ID NO: 24 or a conservative modification thereof, a CDR2 comprising the amino acid sequence set forth in SEQ ID NO: 49 or a conservative modification thereof, a CDR3 comprising the amino acid sequence set forth in SEQ ID NO: 26 or a conservative modification thereof; and a V_{L} comprising a CDR1 comprising the amino acid sequence set forth in SEQ ID NO: 20 or a conservative modification thereof, a CDR2 comprising the amino acid sequence set forth in SEQ ID NO: 28 or a conservative modification thereof, and a CDR3 comprising the amino acid sequence set forth in SEQ ID NO: 50 or a conservative modification.

In certain embodiments, the anti-CD3 scFv comprises a V_{H} comprising a CDR1 comprising the amino acid sequence set forth in SEQ ID NO: 24, a CDR2 comprising the amino acid sequence set forth in SEQ ID NO: 49, a CDR3 comprising the amino acid sequence set forth in SEQ ID NO: 26; and a V_{L} comprising a CDR1 comprising the amino acid sequence set forth in SEQ ID NO: 20, a CDR2 comprising the amino acid sequence set forth in SEQ ID NO: 28, and a CDR3 comprising the amino acid sequence set forth in SEQ ID NO: 50.

In certain embodiments, the anti-CD3 scFv comprises a V_{H} comprising the amino acid sequence set forth in SEQ ID NO: 51, and a V_{L} comprising the amino acid sequence set forth in SEQ ID NO: 52. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 8
(09B01)

| CDRs | 1 | 2 | 3 |
|---|---|---|---|
| V_{H} | GFTFSRFN [SEQ ID | ISSSSSYI [SEQ ID NO: | AGGGYFDY [SEQ ID |
| | NO: 24] | 49] | NO: 26] |
| V_{L} | QILLHSNGYNY [SEQ | LGS [SEQ ID NO: 28] | RQALQTPYT [SEQ ID |
| | ID NO: 20] | | NO: 50] |

| | |
|---|---|
| Full V_{H} | EVQLVESGGGLVKPGGSLRLSCAASGFTFSRFNMNWVRQTPGKGLEWV |
| | SSISSSSSYIYYADSVKGRFTISRDNAKKSLYLQMNSLRAEDTAVYYCAG |
| | GGYFDYWGQGTLVTVSS [SEQ ID NO: 51] |
| Full V_{L} | DIVMTQSPLSLPVTPGEPASISCRSSQILLHSNGYNYLDWYLQKPGQSPQL |
| | LIYLGSNRASGVPDRFSGSGSGTDFTLKISRVEAEDVGVFYCRQALQTPY |
| | TFGQGTKLEIK [SEQ ID NO: 52] |

TABLE 9
(09C02)

| CDRs | 1 | 2 | 3 |
|---|---|---|---|
| V_{H} | GFTFSRFN [SEQ ID | ISSSSSYI [SEQ ID NO: | AGGGYFDY [SEQ ID |
| | NO: 24] | 49] | NO: 26] |
| V_{L} | QILLHSNGYNY [SEQ | LGS [SEQ ID NO: 28] | RQALQTPYT [SEQ ID |
| | ID NO: 20] | | NO: 50] |

| | |
|---|---|
| Full V_{H} | EVQLVESGGGLVKPGGSLRLSCAASGFTFSRFNMNWVRQTPGKGLEWV |
| | SSISSSSSYIYYADSVKGRFTISRDNAKKSLYLQMNSLRAEDTAVYYCAG |
| | GGYFDYWGQGTLVTVSS [SEQ ID NO: 51] |
| Full V_{L} | DIVMTQSPLSLPVTPGEPASISCRSSQILLHSNGYNYLDWYLQKPGQSPQL |
| | LIYLGSNRASGVPDRFSGSGSGTDFTLKISRVEAEDVGVFYCRQALQTPY |
| | TFGQGTKLEIK [SEQ ID NO: 52] |

In certain embodiments, the anti-CD3 antibody is an scFv, an scFv-Fc fusion protein or a full-length human IgG (or antibody fragment thereof) with V_{H} and V_{L} regions or CDRs selected from Table 10. In certain embodiments, the anti-CD3 scFv comprises a V_{H} comprising the amino acid sequence set forth in SEQ ID NO: 54. In certain embodiments, the anti-CD3 scFv comprises a V_{L} comprising the amino acid sequence set forth in SEQ ID NO: 55. In certain embodiments, the anti-CD3 scFv comprises a V_{H} comprising the amino acid sequence set forth in SEQ ID NO: 54 and a V_{L} comprising the amino acid sequence set forth in SEQ ID NO: 55. SEQ ID NO: 54 and 53 are provided in Table 10. In certain embodiments, the scFv is designated as “11A10”.

In certain embodiments, the anti-CD3 scFv comprises a V_{H} comprising a CDR1 comprising the amino acid sequence set forth in SEQ ID NO: 9 or a conservative modification thereof, a CDR2 comprising the amino acid sequence set forth in SEQ ID NO: 18 or a conservative modification thereof, and a CDR3 comprising the amino acid sequence set forth in SEQ ID NO: 46 or a conservative modification thereof. SEQ ID NOs: 9, 18, and 46 are provided in Table 10.

In certain embodiments, the anti-CD3 scFv comprises a V_{L} comprising a CDR1 comprising the amino acid sequence set forth in SEQ ID NO: 53 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof. SEQ ID NOs: 13, 21, and 53 are provided in Table 10.

In certain embodiments, the anti-CD3 scFv comprises a V_{H} comprising a CDR1 comprising the amino acid sequence set forth in SEQ ID NO: 9 or a conservative modification thereof, a CDR2 comprising the amino acid sequence set forth in SEQ ID NO: 18 or a conservative modification thereof, a CDR3 comprising the amino acid sequence set forth in SEQ ID NO: 46 or a conservative modification thereof; and a V_{L} comprising a CDR1 comprising the amino acid sequence set forth in SEQ ID NO: 53 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification.

In certain embodiments, the anti-CD3 scFv comprises a V_{H} comprising a CDR1 comprising the amino acid sequence set forth in SEQ ID NO: 9, a CDR2 comprising the amino acid sequence set forth in SEQ ID NO: 18, a CDR3 comprising the amino acid sequence set forth in SEQ ID NO: 46; and a V_{L} comprising a CDR1 comprising the amino acid sequence set forth in SEQ ID NO: 53, a CDR2 comprising the amino acid sequence set forth in SEQ ID NO: 13, and a CDR3 comprising the amino acid sequence set forth in SEQ ID NO: 21.

In certain embodiments, the anti-CD3 scFv comprises a V_{H} comprising the amino acid sequence set forth in SEQ ID NO: 54, and a V_{L} comprising the amino acid sequence set forth in SEQ ID NO: 55. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 10
(11A10)

| CDRs | 1 | 2 | 3 |
|---|---|---|---|
| V_{H} | GFTFSSYW [SEQ ID | IKEDGSEK [SEQ ID | ARERREGYFDY [SEQ |
| | NO: 9] | NO: 18] | ID NO: 46] |
| V_{L} | QSISSW [SEQ ID NO: | KAS [SEQ ID NO: 13] | QQYKSYSWT [SEQ ID |
| | 53] | | NO: 21] |

| | |
|---|---|
| Full V_{H} | EVQLVESGRGLVQPGGSLRLSCAASGFTFSSYWMSWVRQAPGKGLEWV |
| | ANIKEDGSEKYYVDSVKGRFTISRDNAKNSLYLQMNSLRAEDTAVYYC |
| | ARERREGYFDYWGQGTLVTVSS [SEQ ID NO: 54] |
| Full V_{L} | DIQMTQSPSTLSASVGDRVTITCRASQSISSWLAWYQQKPGKAPDLLIYK |
| | ASSLESGVPSRFSGSGSGTEFTLTISSLQPDDFATYYCQQYKSYSWTFGQ |
| | GTKVEIK [SEQ ID NO: 55] |

In certain embodiments, the anti-CD3 antibody is an scFv, an scFv-Fc fusion protein or a full-length human IgG (or antibody fragment thereof) with V_{H} and V_{L} regions or CDRs selected from Table 11. In certain embodiments, the anti-CD3 scFv comprises a V_{H} comprising the amino acid sequence set forth in SEQ ID NO: 56. In certain embodiments, the anti-CD3 scFv comprises a V_{L} comprising the amino acid sequence set forth in SEQ ID NO: 57. In certain embodiments, the anti-CD3 scFv comprises a V_{H} comprising the amino acid sequence set forth in SEQ ID NO: 56 and a V_{L} comprising the amino acid sequence set forth in SEQ ID NO: 57. SEQ ID NO: 56 and 57 are provided in Table 11. In certain embodiments, the scFv is designated as “11B11”.

In certain embodiments, the anti-CD3 scFv comprises a V_{H} comprising a CDR1 comprising the amino acid sequence set forth in SEQ ID NO: 9 or a conservative modification thereof, a CDR2 comprising the amino acid sequence set forth in SEQ ID NO: 18 or a conservative modification thereof, and a CDR3 comprising the amino acid sequence set forth in SEQ ID NO: 46 or a conservative modification thereof. SEQ ID NOs: 9, 18, and 46 are provided in Table 11.

In certain embodiments, the anti-CD3 scFv comprises a V_{L} comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof. SEQ ID NOs: 12, 13, and 21 are provided in Table 11.

In certain embodiments, the anti-CD3 scFv comprises a V_{H} comprising a CDR1 comprising the amino acid sequence set forth in SEQ ID NO: 9 or a conservative modification thereof, a CDR2 comprising the amino acid sequence set forth in SEQ ID NO: 18 or a conservative modification thereof, a CDR3 comprising the amino acid sequence set forth in SEQ ID NO: 46 or a conservative modification thereof; and a V_{L} comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification.

In certain embodiments, the anti-CD3 scFv comprises a V_{H} comprising a CDR1 comprising the amino acid sequence set forth in SEQ ID NO: 9, a CDR2 comprising the amino acid sequence set forth in SEQ ID NO: 18, a CDR3 comprising the amino acid sequence set forth in SEQ ID NO: 46; and a V_{L} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 21.

In certain embodiments, the anti-CD3 scFv comprises a V_{H} comprising the amino acid sequence set forth in SEQ ID NO: 56, and a V_{L} comprising the amino acid sequence set forth in SEQ ID NO: 57. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 11
(11B11)

| CDRs | 1 | 2 | 3 |
|---|---|---|---|
| V_{H} | GFTFSSYW [SEQ ID | IKEDGSEK [SEQ ID | ARERREGYFDY [SEQ |
| | NO: 9] | NO: 18] | ID NO: 46] |
| V_{L} | QRISTW [SEQ ID NO: | KAS [SEQ ID NO: 13] | QQYKSYSWT [SEQ ID |
| | 12] | | NO: 21] |

| | |
|---|---|
| Full V_{H} | EVQLVESGGGLVQPGGSLRLSCAASGFTFSSYWMTWVRQAPGKGLEWV |
| | ANIKEDGSEKYYVDSVKGRFTISRDNAKNSLYVQMNSLRAEDTAIYYCA |
| | RERREGYFDYWGQGTLVTVSS [SEQ ID NO: 56] |
| Full V_{L} | DIQMTQSPSILSASVGDRATITCRASQRISTWLAWFQQKPGKAPKLLIYK |
| | ASSLESGVPSRFSGSGSGTEFTLTISSLQPDDFATYYCQQYKSYSWTFGQ |
| | GTKVEIK [SEQ ID NO: 57] |

In certain embodiments, the anti-CD3 antibody is an scFv, an scFv-Fc fusion protein or a full-length human IgG (or antibody fragment thereof) with V_{H} and V_{L} regions or CDRs selected from Table 12. In certain embodiments, the anti-CD3 scFv comprises a V_{H} comprising the amino acid sequence set forth in SEQ ID NO: 59. In certain embodiments, the anti-CD3 scFv comprises a V_{L} comprising the amino acid sequence set forth in SEQ ID NO: 60. In certain embodiments, the anti-CD3 scFv comprises a V_{H} comprising the amino acid sequence set forth in SEQ ID NO: 59 and a V_{L} comprising the amino acid sequence set forth in SEQ ID NO: 60. SEQ ID NO: 59 and 60 are provided in Table 12. In certain embodiments, the scFv is designated as “13E03”.

In certain embodiments, the anti-CD3 scFv comprises a V_{H} comprising a CDR1 comprising the amino acid sequence set forth in SEQ ID NO: 9 or a conservative modification thereof, a CDR2 comprising the amino acid sequence set forth in SEQ ID NO: 18 or a conservative modification thereof, and a CDR3 comprising the amino acid sequence set forth in SEQ ID NO: 46 or a conservative modification thereof. SEQ ID NOs: 9, 18, and 46 are provided in Table 12.

In certain embodiments, the anti-CD3 scFv comprises a V_{L} comprising a CDR1 comprising the amino acid sequence set forth in SEQ ID NO: 58 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof. SEQ ID NOs: 13, 21, and 58 are provided in Table 12.

In certain embodiments, the anti-CD3 scFv comprises a V_{H} comprising a CDR1 comprising the amino acid sequence set forth in SEQ ID NO: 9 or a conservative modification thereof, a CDR2 comprising the amino acid sequence set forth in SEQ ID NO: 18 or a conservative modification thereof, a CDR3 comprising the amino acid sequence set forth in SEQ ID NO: 46 or a conservative modification thereof; and a V_{L} comprising a CDR1 comprising the amino acid sequence set forth in SEQ ID NO: 58 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification.

In certain embodiments, the anti-CD3 scFv comprises a V_{H} comprising a CDR1 comprising the amino acid sequence set forth in SEQ ID NO: 9, a CDR2 comprising the amino acid sequence set forth in SEQ ID NO: 18, a CDR3 comprising the amino acid sequence set forth in SEQ ID NO: 46; and a V_{L} comprising a CDR1 comprising the amino acid sequence set forth in SEQ ID NO: 58, a CDR2 comprising the amino acid sequence set forth in SEQ ID NO: 13, and a CDR3 comprising the amino acid sequence set forth in SEQ ID NO: 21.

In certain embodiments, the anti-CD3 scFv comprises a V_{H} comprising the amino acid sequence set forth in SEQ ID NO: 59, and a V_{L} comprising the amino acid sequence set forth in SEQ ID NO: 60. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 12
(13E03)

| CDRs | 1 | 2 | 3 |
|---|---|---|---|
| V_{H} | GFTFSSYW [SEQ ID | IKEDGSEK [SEQ ID | ARERREGYFDY [SEQ |
| | NO: 9] | NO: 18] | ID NO: 46] |
| V_{L} | QSIGRW [SEQ ID NO: | KAS [SEQ ID NO: 13] | QQYKSYSWT [SEQ ID |
| | 58] | | NO: 21] |

| | |
|---|---|
| Full V_{H} | EIQLVESGGGLVQPGGSLRLSCAASGFTFSSYWMSWVRQTPGKGLEWV |
| | ANIKEDGSEKYHVDSVKGRYTISRDNAKNSLYLQMSSLRADDTAVYYC |
| | ARERREGYFDYWGQGTVVTASS [SEQ ID NO: 59] |
| Full V_{L} | DIQMTQSPSTLSASVGDRVTITCRASQSIGRWLAWYQQKPGKAPDLLIYK |
| | ASSLESGVPSRFSGGGSGTEFTLTISSLQPDDFATYYCQQYKSYSWTFGQ |
| | GTKVEVK [SEQ ID NO: 60] |

In certain embodiments, the anti-CD3 antibody is an scFv, an scFv-Fc fusion protein or a full-length human IgG (or antibody fragment thereof) with V_{H} and V_{L} regions or CDRs selected from Table 13 or Table 14. In certain embodiments, the anti-CD3 scFv comprises a V_{H} comprising the amino acid sequence set forth in SEQ ID NO: 66. In certain embodiments, the anti-CD3 scFv comprises a V_{L} comprising the amino acid sequence set forth in SEQ ID NO: 67. In certain embodiments, the anti-CD3 scFv comprises a V_{H} comprising the amino acid sequence set forth in SEQ ID NO: 66 and a V_{L} comprising the amino acid sequence set forth in SEQ ID NO: 67. SEQ ID NO: 66 and 67 are provided in Table 13. In certain embodiments, the scFv is designated as “07B10” or “07B12”.

In certain embodiments, the anti-CD3 scFv comprises a V_{H} comprising a CDR1 comprising the amino acid sequence set forth in SEQ ID NO: 61 or a conservative modification thereof, a CDR2 comprising the amino acid sequence set forth in SEQ ID NO: 62 or a conservative modification thereof, and a CDR3 comprising the amino acid sequence set forth in SEQ ID NO: 63 or a conservative modification thereof. SEQ ID NOs: 61-63 are provided in Table 13 or Table 14.

In certain embodiments, the anti-CD3 scFv comprises a V_{L} comprising a CDR1 comprising the amino acid sequence set forth in SEQ ID NO: 4 or a conservative modification thereof, a CDR2 comprising the amino acid sequence set forth in SEQ ID NO: 64 or a conservative modification thereof, and a CDR3 comprising the amino acid sequence set forth in SEQ ID NO: 65 or a conservative modification thereof. SEQ ID NOs: 4, 64, and 65 are provided in Table 13 or Table 14.

In certain embodiments, the anti-CD3 scFv comprises a V_{H} comprising a CDR1 comprising the amino acid sequence set forth in SEQ ID NO: 61 or a conservative modification thereof, a CDR2 comprising the amino acid sequence set forth in SEQ ID NO: 62 or a conservative modification thereof, a CDR3 comprising the amino acid sequence set forth in SEQ ID NO: 63 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 64 or a conservative modification thereof, and a CDR3 comprising the amino acid sequence set forth in SEQ ID NO: 65 or a conservative modification.

In certain embodiments, the anti-CD3 scFv comprises a V_{H} comprising a CDR1 comprising the amino acid sequence set forth in SEQ ID NO: 61, a CDR2 comprising the amino acid sequence set forth in SEQ ID NO: 62, a CDR3 comprising the amino acid sequence set forth in SEQ ID NO: 63; and a V_{L} comprising a CDR1 comprising the amino acid sequence set forth in SEQ ID NO: 4, a CDR2 comprising the amino acid sequence set forth in SEQ ID NO: 64, and a CDR3 comprising the amino acid sequence set forth in SEQ ID NO: 65.

In certain embodiments, the anti-CD3 scFv comprises a V_{H} comprising the amino acid sequence set forth in SEQ ID NO: 66, and a V_{L} comprising the amino acid sequence set forth in SEQ ID NO: 67. In certain embodiments, the V_{H} and V_{L} are linked via a linker. In certain embodiments, the linker comprises the amino acid sequence set forth in SEQ ID NO: 75, SEQ ID NO: 76, SEQ ID NO: 77, SEQ ID NO: 78, SEQ ID NO: 79, or SEQ ID NO: 80.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 13
(07B10)

| CDRs | 1 | 2 | 3 |
|---|---|---|---|
| V_{H} | GYTFTDYY | INPYRGAI [SEQ ID | ARDGGYCSGGSCYKGA |
| | [SEQ ID NO: 61] | NO: 62] | FDI [SEQ ID NO: 63] |
| VI | QSLVHSDGNTY | KIS [SEQ ID NO: 64] | MQATHFPHT [SEQ ID |
| | [SEQ ID NO: 4] | | NO: 65] |

| | |
|---|---|
| Full V_{H} | QVQLVQSGAEVKKPGASVKVSCKASGYTFTDYYMHWVRQSPGQGLEW |
| | MGWINPYRGAINYAQMFQGRVTMTRDTSISTAYMELSRLNSDDTAVYY |
| | CARDGGYCSGGSCYKGAFDIWGQGTMVTVSS [SEQ ID NO: 66] |
| Full V_{L} | DIVMTQTPLSSPVTLGQPASISCRSSQSLVHSDGNTYLSWLQQRPGQPPR |
| | LLIYKISNRFSGVPDRFSGSGAGTDFTLKISRVEPEDVGVYYCMQATHFP |
| | HTFGQGTRLEIK [SEQ ID NO: 67] |

TABLE 14
(07B12)

| CDRs | 1 | 2 | 3 |
|---|---|---|---|
| V_{H} | GYTFTDYY | INPYRGAI [SEQ ID | ARDGGYCSGGSCYKGA |
| | [SEQ ID NO: 61] | NO: 62] | FDI [SEQ ID NO: 63] |
| V_{L} | QSLVHSDGNTY | KIS [SEQ ID NO: 64] | MQATHFPHT [SEQ ID |
| | [SEQ ID NO: 4] | | NO: 65] |

| | |
|---|---|
| Full V_{H} | QVQLVQSGAEVKKPGASVKVSCKASGYTFTDYYMHWVRQSPGQGLEW |
| | MGWINPYRGAINYAQMFQGRVTMTRDTSISTAYMELSRLNSDDTAVYY |
| | CARDGGYCSGGSCYKGAFDIWGQGTMVTVSS [SEQ ID NO: 66] |
| Full V_{L} | DIVMTQTPLSSPVTLGQPASISCRSSQSLVHSDGNTYLSWLQQRPGQPPR |
| | LLIYKISNRFSGVPDRFSGSGAGTDFTLKISRVEPEDVGVYYCMQATHFP |
| | HTFGQGTRLEIK [SEQ ID NO: 67] |

In certain embodiments, the anti-CD3 antibody is an antibody fragment with V_{H} region or CDRs selected from Table 15. In certain embodiments, the anti-CD3 antibody fragment comprises a V_{H} comprising the amino acid sequence set forth in SEQ ID NO: 70. SEQ ID NO: 70 is provided in Table 15. In certain embodiments, the antibody fragment is designated as “08B12”.

In certain embodiments, the anti-CD3 antibody fragment comprises a V_{H} comprising a CDR1 comprising the amino acid sequence set forth in SEQ ID NO: 68 or a conservative modification thereof, a CDR2 comprising the amino acid sequence set forth in SEQ ID NO: 69 or a conservative modification thereof, and a CDR3 comprising the amino acid sequence set forth in SEQ ID NO: 26 or a conservative modification thereof. SEQ ID NOs: 26, 68, and 69 are provided in Table 15.

In certain embodiments, the anti-CD3 antibody fragment comprises a V_{H} comprising a CDR1 comprising the amino acid sequence set forth in SEQ ID NO: 68, a CDR2 comprising the amino acid sequence set forth in SEQ ID NO: 69, and a CDR3 comprising the amino acid sequence set forth in SEQ ID NO: 26.

TABLE 15
(Clone: 08B12)

| CDRs | 1 | 2 | 3 |
|---|---|---|---|
| V_{H} | GFTFKKYN [SEQ ID | ISSSSTYI[SEQ ID NO: | AGGGYFDY[SEQ ID |
| | NO: 68] | 69] | NO: 26] |

| | |
|---|---|
| Full V_{H} | EVQVVESGGGLVKPGGSLRLSCVASGFTFKKYNMNWVRQAPGKGLEW |
| | VSSISSSSTYIYYADSVKGRFTISRDNAKNSLNLQMNSLRAEDTAVYYCA |
| | GGGYFDYWGQGTLVTVSS [SEQ ID NO: 70] |

### 3.2. Monoclonal Antibodies

The presently disclosed subject matter provides antibodies (e.g., human antibodies, e.g., human monoclonal antibodies) that specifically bind to CD3ε (e.g., human CD3E). The V_{H} amino acid sequences of anti-CD3 antibodies 01F05G01, 07B09, 12C08, 13D02, 01B08B01, 06G07A04, 08G12, 09B01, 09C02, 11A10, 11B11, 13E03, 07B10, 07B12, and 08B12 are set forth in SEQ ID NOs: 7, 15, 22, 30, 38, 43, 47, 51, 54, 56, 59, 66, and 70. The V_{L} amino acid sequences of 01F05G01, 07B09, 12C08, 13D02, 01B08B01, 06G07A04, 08G12, 09B01, 09C02, 11A10, 11B11, 13E03, 07B10, 07B 12, and 08B12 are set forth in SEQ ID NOs: 8, 16, 23, 31, 39, 44, 48, 52, 55, 57, 60, and 67.

Given that each of 01F05G01, 07B09, 12C08, 13D02, 01B08B01, 06G07A04, 08G12, 09B01, 09C02, 11A10, 11B11, 13E03, 07B10, 07B12, and 08B 12 antibodies can bind to CD3, the V_{H} and V_{L} sequences can be “mixed and matched” to create other anti-CD3 binding molecules. CD3 binding of such “mixed and matched” antibodies can be tested using the binding assays known in the art, including for example, ELISAs, Western blots, RIAs, Biacore analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}/V_{L} pairing is replaced with a structurally similar V_{L} sequence.

In certain embodiments, the presently disclosed subject matter provides an antibody or an antigen-binding fragment thereof comprising: (a) a heavy chain variable region (V_{H}) comprising an amino acid sequence selected from SEQ ID NOs: 7, 15, 22, 30, 38, 43, 47, 51, 54, 56, 59, 66, and 70; and (b) a light chain variable region (V_{L}) comprising an amino acid sequence selected from SEQ ID NOs: 8, 16, 23, 31, 39, 44, 48, 52, 55, 57, 60, and 67; wherein the antibody or antigen-binding fragment specifically binds to CD38, e.g., human CD38. In certain embodiments, the V_{H} and V_{L} are selected from the group consisting of:
(a) a heavy chain variable region comprising the amino acid sequence set forth in SEQ ID NO: 8, and a light chain variable region comprising the amino acid sequence set forth in SEQ ID NO: 8;
(b) a heavy chain variable region comprising the amino acid sequence set forth in SEQ ID NO: 15, and a light chain variable region comprising the amino acid sequence set forth in SEQ ID NO: 16;
(c) a heavy chain variable region comprising the amino acid sequence set forth in SEQ ID NO: 22, and a light chain variable region comprising the amino acid sequence set forth in SEQ ID NO: 23;
(d) a heavy chain variable region comprising the amino acid sequence set forth in SEQ ID NO: 30, and a light chain variable region comprising the amino acid sequence set forth in SEQ ID NO: 31;
(e) a heavy chain variable region comprising the amino acid sequence set forth in SEQ ID NO: 38, and a light chain variable region comprising the amino acid sequence set forth in SEQ ID NO: 39;
(f) a heavy chain variable region comprising the amino acid sequence set forth in SEQ ID NO: 43, and a light chain variable region comprising the amino acid sequence set forth in SEQ ID NO: 44;
(g) a heavy chain variable region comprising the amino acid sequence set forth in SEQ ID NO: 47, and a light chain variable region comprising the amino acid sequence set forth in SEQ ID NO: 48;
(h) a heavy chain variable region comprising the amino acid sequence set forth in SEQ ID NO: 51, and a light chain variable region comprising the amino acid sequence set forth in SEQ ID NO: 52;
(i) a heavy chain variable region comprising the amino acid sequence set forth in SEQ ID NO: 54, and a light chain variable region comprising the amino acid sequence set forth in SEQ ID NO: 55;
(j) a heavy chain variable region comprising the amino acid sequence set forth in SEQ ID NO: 56, and a light chain variable region comprising the amino acid sequence set forth in SEQ ID NO: 27; and
(k) a heavy chain variable region comprising the amino acid sequence set forth in SEQ ID NO: 59, and a light chain variable region comprising the amino acid sequence set forth in SEQ ID NO: 60; and
(l) a heavy chain variable region comprising the amino acid sequence set forth in SEQ ID NO: 66, and a light chain variable region comprising the amino acid sequence set forth in SEQ ID NO: 67.

In certain embodiments, the presently disclosed subject matter provides antibodies or antigen-binding fragments thereof that comprise the heavy chain and light chain CDR1s, CDR2s and CDR3s of 01F05G01, 07B09, 12C08, 13D02, 01B08B01, 06G07A04, 08G12, 09B01, 09C02, 11A10, 11B11, 13E03, 07B10, 07B 12, and 08B12.

The amino acid sequences of the V_{H} CDR1s of 01F05G01, 07B09, 12C08, 13D02, 01B08B01, 06G07A04, 08G12, 09B01, 09C02, 11A10, 11B11, 13E03, 07B10, 07B12, and 08B12 are set forth in SEQ ID NOs: 1, 9, 17, 24, 32, 40, 45, 61, and 68. The amino acid sequences of the V_{H} CDR2s of 01F05G01, 07B09, 12C08, 13D02, 01B08B01, 06G07A04, 08G12, 09B01, 09C02, 11A10, 11B11, 13E03, 07B10, 07B12, and 08B12 antibodies are set forth in SEQ ID NOs: 2, 10, 18, 25, 33, 49, 62, and 69. The amino acid sequences of the V_{H} CDR3s of 01F05G01, 07B09, 12C08, 13D02, 01B08B01, 06G07A04, 08G12, 09B01, 09C02, 11A10, 11B11, 13E03, 07B 10, 07B12, and 08B12 are set forth in SEQ ID NOs: 3, 11, 19, 26, 34, 41, 46, and 63.

The amino acid sequences of the V_{L} CDR1s of 01F05G01, 07B09, 12C08, 13D02, 01B08B01, 06G07A04, 08G12, 09B01, 09C02, 11A10, 11B11, 13E03, 07B10, and 07B12 are set forth in SEQ ID NOs: 4, 12, 20, 27, 35, 42, 53, and 58. The amino acid sequences of the V_{L} CDR2s of 01F05G01, 07B09, 12C08, 13D02, 01B08B01, 06G07A04, 08G12, 09B01, 09C02, 11A10, 11B11, 13E03, 07B10, and 07B 12 are set forth in SEQ ID NOs: 5, 13, 28, 36, and 64. The amino acid sequences of the V_{L} CDR3s of 01F05G01, 07B09, 12C08, 13D02, 01B08B01, 06G07A04, 08G12, 09B01, 09C02, 11A10, 11B11, 13E03, 07B 10, and 07B 12 are set forth in SEQ ID NOs: 6, 14, 21, 29, 37, 50, and 65. The CDR regions are delineated using the IMGT system. In certain embodiments, the CDR regions are delineated using the IMGT numbering system accessible at https://www.imgt.org/IMGTScientificChart/Nomenclature/IMGT-FRCDRdefinition.html.

Given that each of these antibodies or antigen-binding fragments thereof can bind to CD3ε and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V_{L} CDR1, CDR2, and CDR3) to create other anti-CD3 binding molecules. CD3 binding of such “mixed and matched” antibodies can be tested using the binding assays described above. When V_{H} CDR sequences are mixed and matched, the CDR1, CDR2 and/or CDR3 sequence from a particular V_{H} sequence is replaced with a structurally similar CDR sequence(s). Likewise, when V_{L} CDR sequences are mixed and matched, the CDR1, CDR2 and/or CDR3 sequence from a particular V_{L} sequence is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies or antigen-binding fragments thereof disclosed herein 01F05G01, 07B09, 12C08, 13D02, 01B08B01, 06G07A04, 08G12, 09B01, 09C02, 11A10, 11B11, 13E03, 07B10, 07B12, and 08B 12.

In certain embodiments, the presently disclosed subject matter provides an anti-CD3 antibody or an antigen-binding fragment thereof comprising:
(a) a heavy chain variable region CDR1 comprising the amino acid sequence set forth in SEQ ID NO: 1, SEQ ID NO: 9, SEQ ID NO: 17, SEQ ID NO: 24, SEQ ID NO: 32, SEQ ID NO: 40, SEQ ID NO: 45, SEQ ID NO: 61, or SEQ ID NO: 68;
(b) a heavy chain variable region CDR2 comprising the amino acid sequence set forth in SEQ ID NO: 2, SEQ ID NO: 10, SEQ ID NO: 18, SEQ ID NO: 25, SEQ ID NO: 33, SEQ ID NO: 49, SEQ ID NO: 62, or SEQ ID NO: 69;
(c) a heavy chain variable region CDR3 comprising the amino acid sequence set forth in SEQ ID NO: 3, SEQ ID NO: 11, SEQ ID NO: 19, SEQ ID NO: 26, SEQ ID NO: 34, SEQ ID NO: 41, SEQ ID NO: 46, or SEQ ID NO: 63;
(d) a light chain variable region CDR1 comprising the amino acid sequence set forth in SEQ ID NO: 4, SEQ ID NO: 12, SEQ ID NO: 20, SEQ ID NO: 27, SEQ ID NO: 35, SEQ ID NO: 42, SEQ ID NO: 53, or SEQ ID NO: 58;
(e) a light chain variable region CDR2 comprising the amino acid sequence set forth in SEQ ID NO: 5, SEQ ID NO: 13, SEQ ID NO: 28, SEQ ID NO: 36, or SEQ ID NO: 64; and
(f) a light chain variable region CDR3 comprising the amino acid sequence set forth in SEQ ID NO: 6, SEQ ID NO: 14, SEQ ID NO: 21, SEQ ID NO: 29, SEQ ID NO: 37, SEQ ID NO: 50, or SEQ ID NO: 65.

In certain embodiments, the anti-CD3 antibody or antigen-binding fragment thereof comprises:
(a) a heavy chain variable region CDR1 comprising the amino acid sequence set forth in SEQ ID NO: 1;
(b) a heavy chain variable region CDR2 comprising the amino acid sequence set forth in SEQ ID NO: 2;
(c) a heavy chain variable region CDR3 comprising the amino acid sequence set forth in SEQ ID NO: 3;
(d) a light chain variable region CDR1 comprising the amino acid sequence set forth in SEQ ID NO: 4;
(e) a light chain variable region CDR2 comprising the amino acid sequence set forth in SEQ ID NO: 5; and
(f) a light chain variable region CDR3 comprising the amino acid sequence set forth in SEQ ID NO: 6.

In certain embodiments, the anti-CD3 antibody or antigen-binding fragment thereof comprises:
(a) a heavy chain variable region CDR1 comprising the amino acid sequence set forth in SEQ ID NO: 9;
(b) a heavy chain variable region CDR2 comprising the amino acid sequence set forth in SEQ ID NO: 10;
(c) a heavy chain variable region CDR3 comprising the amino acid sequence set forth in SEQ ID NO: 11;
(d) a light chain variable region CDR1 comprising the amino acid sequence set forth in SEQ ID NO: 12;
(e) a light chain variable region CDR2 comprising the amino acid sequence set forth in SEQ ID NO: 13; and
(f) a light chain variable region CDR3 comprising the amino acid sequence set forth in SEQ ID NO: 14.

In certain embodiments, the anti-CD3 antibody or antigen-binding fragment thereof comprises:
(a) a heavy chain variable region CDR1 comprising the amino acid sequence set forth in SEQ ID NO: 17;
(b) a heavy chain variable region CDR2 comprising the amino acid sequence set forth in SEQ ID NO: 18;
(c) a heavy chain variable region CDR3 comprising the amino acid sequence set forth in SEQ ID NO: 19;
(d) a light chain variable region CDR1 comprising the amino acid sequence set forth in SEQ ID NO: 20;
(e) a light chain variable region CDR2 comprising the amino acid sequence set forth in SEQ ID NO: 13; and
(f) a light chain variable region CDR3 comprising the amino acid sequence set forth in SEQ ID NO: 21.

In certain embodiments, the anti-CD3 antibody or antigen-binding fragment thereof comprises:
(a) a heavy chain variable region CDR1 comprising the amino acid sequence set forth in SEQ ID NO: 24;
(b) a heavy chain variable region CDR2 comprising the amino acid sequence set forth in SEQ ID NO: 25;
(c) a heavy chain variable region CDR3 comprising the amino acid sequence set forth in SEQ ID NO: 26;
(d) a light chain variable region CDR1 comprising the amino acid sequence set forth in SEQ ID NO: 27;
(e) a light chain variable region CDR2 comprising the amino acid sequence set forth in SEQ ID NO: 28; and
(f) a light chain variable region CDR3 comprising the amino acid sequence set forth in SEQ ID NO: 29.

In certain embodiments, the anti-CD3 antibody or antigen-binding fragment thereof comprises:
(a) a heavy chain variable region CDR1 comprising the amino acid sequence set forth in SEQ ID NO: 32;
(b) a heavy chain variable region CDR2 comprising the amino acid sequence set forth in SEQ ID NO: 33;
(c) a heavy chain variable region CDR3 comprising the amino acid sequence set forth in SEQ ID NO: 34;
(d) a light chain variable region CDR1 comprising the amino acid sequence set forth in SEQ ID NO: 35;
(e) a light chain variable region CDR2 comprising the amino acid sequence set forth in SEQ ID NO: 36; and
(f) a light chain variable region CDR3 comprising the amino acid sequence set forth in SEQ ID NO: 37.

In certain embodiments, the anti-CD3 antibody or antigen-binding fragment thereof comprises:
(a) a heavy chain variable region CDR1 comprising the amino acid sequence set forth in SEQ ID NO: 40;
(b) a heavy chain variable region CDR2 comprising the amino acid sequence set forth in SEQ ID NO: 18;
(c) a heavy chain variable region CDR3 comprising the amino acid sequence set forth in SEQ ID NO: 41;
(d) a light chain variable region CDR1 comprising the amino acid sequence set forth in SEQ ID NO: 42;
(e) a light chain variable region CDR2 comprising the amino acid sequence set forth in SEQ ID NO: 13; and
(f) a light chain variable region CDR3 comprising the amino acid sequence set forth in SEQ ID NO: 21.

In certain embodiments, the anti-CD3 antibody or antigen-binding fragment thereof comprises:
(a) a heavy chain variable region CDR1 comprising the amino acid sequence set forth in SEQ ID NO: 45;
(b) a heavy chain variable region CDR2 comprising the amino acid sequence set forth in SEQ ID NO: 18;
(c) a heavy chain variable region CDR3 comprising the amino acid sequence set forth in SEQ ID NO: 46;
(d) a light chain variable region CDR1 comprising the amino acid sequence set forth in SEQ ID NO: 20;
(e) a light chain variable region CDR2 comprising the amino acid sequence set forth in SEQ ID NO: 13; and
(f) a light chain variable region CDR3 comprising the amino acid sequence set forth in SEQ ID NO: 21.

In certain embodiments, the anti-CD3 antibody or antigen-binding fragment thereof comprises:
(a) a heavy chain variable region CDR1 comprising the amino acid sequence set forth in SEQ ID NO: 24;
(b) a heavy chain variable region CDR2 comprising the amino acid sequence set forth in SEQ ID NO: 49;
(c) a heavy chain variable region CDR3 comprising the amino acid sequence set forth in SEQ ID NO: 26;
(d) a light chain variable region CDR1 comprising the amino acid sequence set forth in SEQ ID NO: 20;
(e) a light chain variable region CDR2 comprising the amino acid sequence set forth in SEQ ID NO: 28; and
(f) a light chain variable region CDR3 comprising the amino acid sequence set forth in SEQ ID NO: 50.

In certain embodiments, the anti-CD3 antibody or antigen-binding fragment thereof comprises:
(a) a heavy chain variable region CDR1 comprising the amino acid sequence set forth in SEQ ID NO: 9;
(b) a heavy chain variable region CDR2 comprising the amino acid sequence set forth in SEQ ID NO: 18;
(c) a heavy chain variable region CDR3 comprising the amino acid sequence set forth in SEQ ID NO: 46;
(d) a light chain variable region CDR1 comprising the amino acid sequence set forth in SEQ ID NO: 53;
(e) a light chain variable region CDR2 comprising the amino acid sequence set forth in SEQ ID NO: 13; and
(f) a light chain variable region CDR3 comprising the amino acid sequence set forth in SEQ ID NO: 21.

In certain embodiments, the anti-CD3 antibody or antigen-binding fragment thereof comprises:
(a) a heavy chain variable region CDR1 comprising the amino acid sequence set forth in SEQ ID NO: 9;
(b) a heavy chain variable region CDR2 comprising the amino acid sequence set forth in SEQ ID NO: 18;
(c) a heavy chain variable region CDR3 comprising the amino acid sequence set forth in SEQ ID NO: 46;
(d) a light chain variable region CDR1 comprising the amino acid sequence set forth in SEQ ID NO: 12;
(e) a light chain variable region CDR2 comprising the amino acid sequence set forth in SEQ ID NO: 13; and
(f) a light chain variable region CDR3 comprising the amino acid sequence set forth in SEQ ID NO: 21.

In certain embodiments, the anti-CD3 antibody or antigen-binding fragment thereof comprises:
(a) a heavy chain variable region CDR1 comprising the amino acid sequence set forth in SEQ ID NO: 9;
(b) a heavy chain variable region CDR2 comprising the amino acid sequence set forth in SEQ ID NO: 18;
(c) a heavy chain variable region CDR3 comprising the amino acid sequence set forth in SEQ ID NO: 46;
(d) a light chain variable region CDR1 comprising the amino acid sequence set forth in SEQ ID NO: 58;
(e) a light chain variable region CDR2 comprising the amino acid sequence set forth in SEQ ID NO: 13; and
(f) a light chain variable region CDR3 comprising the amino acid sequence set forth in SEQ ID NO: 21.

In certain embodiments, the anti-CD3 antibody or antigen-binding fragment thereof comprises:
(a) a heavy chain variable region CDR1 comprising the amino acid sequence set forth in SEQ ID NO: 61;
(b) a heavy chain variable region CDR2 comprising the amino acid sequence set forth in SEQ ID NO: 62;
(c) a heavy chain variable region CDR3 comprising the amino acid sequence set forth in SEQ ID NO: 63;
(d) a light chain variable region CDR1 comprising the amino acid sequence set forth in SEQ ID NO: 4;
(e) a light chain variable region CDR2 comprising the amino acid sequence set forth in SEQ ID NO: 64; and
(f) a light chain variable region CDR3 comprising the amino acid sequence set forth in SEQ ID NO: 65.

In certain embodiments, the anti-CD3 antibody or antigen-binding fragment thereof comprises:
(a) a heavy chain variable region CDR1 comprising the amino acid sequence set forth in SEQ ID NO: 68;
(b) a heavy chain variable region CDR2 comprising the amino acid sequence set forth in SEQ ID NO: 69; and
(c) a heavy chain variable region CDR3 comprising the amino acid sequence set forth in SEQ ID NO: 26.

In certain embodiments, the anti-CD3 antibody or antigen-binding fragment thereof comprises a heavy chain constant region and/or a light chain constant region.

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 82. In certain embodiments, the heavy chain constant region comprises the amino acid sequence set forth in SEQ ID NO: 82. SEQ ID NO: 82 is provided below.

[SEQ ID NO: 82]
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 83. In certain embodiments, the heavy chain constant region comprises the amino acid sequence set forth in SEQ ID NO: 83. SEQ ID NO: 83 is provided below.

[SEQ ID NO: 83]
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 84. In certain embodiments, the heavy chain constant region comprises the amino acid sequence set forth in SEQ ID NO: 84. SEQ ID NO: 84 is provided below.

[SEQ ID NO: 84]
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK

In certain embodiments, the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 85. In certain embodiments, the heavy chain constant region comprises the amino acid sequence set forth in SEQ ID NO: 85. SEQ ID NO: 85 is provided below.

[SEQ ID NO: 85]
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK

In certain embodiments, the light chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 86. In certain embodiments, the light chain constant region comprises the amino acid sequence set forth in SEQ ID NO: 86. SEQ ID NO: 86 is provided below.

[SEQ ID NO: 86]
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In certain embodiments, the anti-CD3 antibody or an antigen-binding fragment thereof comprises: (a) a heavy chain constant region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 82, SEQ ID NO: 83, SEQ ID NO: 84, or SEQ ID NO: 85; and (b) a light chain constant region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 86. In certain embodiments, the anti-CD3 antibody or an antigen-binding fragment thereof comprises: (a) a heavy chain constant region comprising the amino acid sequence set forth in SEQ ID NO: 82, SEQ ID NO: 83, SEQ ID NO: 84, or SEQ ID NO: 85; and (b) a light chain constant region comprising the amino acid sequence set forth in SEQ ID NO: 86. In certain embodiments, the anti-CD3 antibody or an antigen-binding fragment thereof comprises: (a) a heavy chain constant region comprising the amino acid sequence set forth in SEQ ID NO: 82; and (b) a light chain constant region comprising the amino acid sequence set forth in SEQ ID NO: 86.

The constant regions/framework regions of the anti-CD3 antibodies disclosed herein can be altered, for example, by amino acid substitution, to modify the properties of the antibody (e.g., to increase or decrease one or more of antigen binding affinity, Fc receptor binding, antibody carbohydrate, for example, glycosylation, fucosylation etc., the number of cysteine residues, effector cell function, effector cell function, complement function or introduction of a conjugation site).

In certain embodiments, a presently disclosed anti-CD3 antibody is a fully-human antibody, e.g., any one of 01F05G01, 07B09, 12C08, 13D02, 01B08B01, 06G07A04, 08G12, 09B01, 09C02, 11A10, 11B11, 13E03, 07B10, 07B12, and 08B12. Fully-human mAbs, when administered to humans, cause serious side effects, including anaphylaxis and hypersensitivity reactions.

The use of phage display libraries has made it possible to select large numbers of antibody repertoires for unique and rare Abs against very defined epitopes (for more details on phage display see McCafferty et al., Phage antibodies: filamentous phage displaying antibody variable domains. Nature, 348:552-554.) The rapid identification of human Fab or single chain Fv (scFv) fragments highly specific for tumor antigen-derived peptide-MHC complex molecules has thus become possible. In addition, by engineering full-length monoclonal antibody (mAb) using the Fab fragments, it is possible to directly generate a therapeutic human mAb, bypassing months of time-consuming work, normally needed for developing therapeutic mAbs. The presently disclosed subject matter involves the development of a fully human mAb that recognizes, for example, a human CD3ε polypeptide (e.g., a polypeptide having the amino acid sequence set forth in SEQ ID NO: 81) for cancer therapy.

### 3.3. Homologous Antibodies

In certain embodiments, a presently disclosed anti-CD3 antibody or antigen-binding fragment thereof comprises heavy and light chain variable regions comprising amino acid sequences that are homologous or identical to the amino acid sequences of the antibodies described herein (e.g., 01F05G01, 07B09, 12C08, 13D02, 01B08B01, 06G07A04, 08G12, 09B01, 09C02, 11A10, 11B11, 13E03, 07B10, 07B12, and 08B12 antibodies), and wherein the antibodies or antigen-binding fragments thereof retain the desired functional properties of the anti-CD3 antibodies or antigen-binding fragments thereof of the presently disclosed subject matter.

For example, the presently disclosed subject matter provides an anti-CD3 antibody or an antigen-binding fragment thereof, comprising a heavy chain variable region and a light chain variable region, wherein:
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 7, SEQ ID NO: 15, SEQ ID NO: 22, SEQ ID NO: 30, SEQ ID NO: 38, SEQ ID NO: 43, SEQ ID NO: 47, SEQ ID NO: 51, SEQ ID NO: 54, SEQ ID NO: 56, SEQ ID NO: 59, SEQ ID NO: 66, or SEQ ID NO: 70; and (b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 16, SEQ ID NO: 23, SEQ ID NO: 31, SEQ ID NO: 39, SEQ ID NO: 44, SEQ ID NO: 48, SEQ ID NO: 52, SEQ ID NO: 55, SEQ ID NO: 57, SEQ ID NO: 60, or SEQ ID NO: 67.

In certain embodiments, the V_{H} and/or V_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having V_{H} and V_{L} regions having high (i.e., 80% or greater) homology or identity to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

In certain embodiments, a presently disclosed anti-CD3 antibody or antigen-binding fragment thereof comprises heavy and light chain constant regions comprising amino acid sequences that are homologous or identical to the amino acid sequences of the antibodies described herein (e.g., 01F05G01, 07B09, 12C08, 13D02, 01B08B01, 06G07A04, 08G12, 09B01, 09C02, 11A10, 11B11, 13E03, 07B10, 07B12, and 08B12 antibodies), and wherein the antibodies or antigen-binding fragments thereof retain the desired functional properties of the anti-CD3 antibodies or antigen-binding fragments thereof of the presently disclosed subject matter.

For example, the presently disclosed subject matter provides an anti-CD3 antibody or an antigen-binding fragment thereof, comprising a heavy chain constant region and a light chain constant region, wherein:
(a) the heavy chain constant region (CH) comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 82; and
(b) the light chain constant region (CL) comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 86.

In certain embodiments, the C_{H} and/or C_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having C_{H} and C_{L} regions having high (i.e., 80% or greater) homology or identity to the C_{H} and C_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity or homology between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology or identity between two amino acid sequences can be determined using the algorithm of E. Meyers and W. Miller (Comput Appl Biosci (1988); 14:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J Mol Biol (1970); 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul et al., J Mol Biol (1990); 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., Nucleic Acids Res (1997); 25 (17): 3389-3402. When utilizing BLAST and Gapped BLAST programs, the default parameters of the respective programs (e.g., XBLAST and NBLAST) can be used.

3.4. Antibodies with Conservative Modifications

In certain embodiments, a presently disclosed anti-CD3 antibody or an antigen-binding fragment thereof comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., 01F05G01, 07B09, 12C08, 13D02, 01B08B01, 06G07A04, 08G12, 09B01, 09C02, 11A10, 11B11, 13E03, 07B10, 07B12, and 08B12 antibodies), or a conservative modification thereof, and wherein the antibodies retain the desired functional properties of the anti-CD3 antibodies or antigen-binding fragments thereof of the presently disclosed subject matter. The presently disclosed subject matter provides an antibody or an antigen-binding fragment thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises an amino acid sequence selected from SEQ ID NOs: 3, 11, 19, 26, 34, 41, 46, and 63, and conservative modifications thereof;
(b) the light chain variable region CDR3 sequence comprises an amino acid sequence selected from SEQ ID NOs: 6, 14, 21, 29, 37, 50, and 65, and conservative modifications thereof.

In certain embodiments, the heavy chain variable region CDR3 sequence comprises an amino acid sequence selected from SEQ ID NOs: 3, 11, 19, 26, 34, 41, 46, and 63, and conservative modifications thereof; and the light chain variable region CDR3 sequence comprises an amino acid sequence selected from SEQ ID NOs: 6, 14, 21, 29, 37, 50, and 65, and conservative modifications thereof.

In certain embodiments, the heavy chain variable region CDR2 sequence comprises an amino acid sequence selected from SEQ ID NOs: 2, 10, 18, 25, 33, 49, 62, and 69, and conservative modifications thereof; and the light chain variable region CDR2 sequence comprises an amino acid sequence selected from SEQ ID NOs: 5, 13, 28, 36, and 64, and conservative modifications thereof.

In certain embodiments, the heavy chain variable region CDR1 sequence comprises an amino acid sequence selected from SEQ ID NOs: 1, 9, 17, 24, 32, 40, 45, 61, and 68, and conservative modifications thereof; and the light chain variable region CDR1 sequence comprises an amino acid sequence selected from SEQ ID NOs: 4, 12, 20, 27, 35, 42, 53, and 58, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions, and deletions. Modifications can be introduced into an antibody of the present disclosure by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 16. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted.

TABLE 16

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

3.5. Anti-CD3 Antibodies that Cross-Compete for Binding to CD3& with Anti-CD3 Antibodies of the Present Disclosure

The presently disclosed subject matter provides antibodies or antigen-binding fragments thereof that cross-compete with any of the disclosed anti-CD3 antibodies for binding to CD3ε (e.g., human CD3ε). For example, and not by way of limitation, the cross-competing antibodies can bind to the same epitope region, e.g., same epitope, adjacent epitope, or overlapping as any of the anti-CD3 antibodies or antigen-binding fragments thereof of the presently disclosed subject matter. In certain embodiments, the reference antibody or reference antigen-binding fragments thereof for cross-competition studies can be any one of the anti-CD3 antibodies or antigen-binding fragments thereof disclosed herein, e.g., 01F05G01, 07B09, 12C08, 13D02, 01B08B01, 06G07A04, 08G12, 09B01, 09C02, 11A10, 11B11, 13E03, 07B 10, 07B12, and 08B 12 antibodies.

Such cross-competing antibodies can be identified based on their ability to cross-compete with any one of the presently disclosed anti-CD3 antibodies or antigen-binding fragments thereof in standard CD3ε binding assays. For example, Biacore analysis, ELISA assays or flow cytometry can be used to demonstrate cross-competition with the antibodies of the presently disclosed subject matter. The ability of a test antibody to inhibit the binding of, for example, any one of the presently disclosed anti-CD3 antibodies (e.g., 01F05G01, 07B09, 12C08, 13D02, 01B08B01, 06G07A04, 08G12, 09B01, 09C02, 11A10, 11B11, 13E03, 07B10, 07B 12, and 08B 12 antibodies) to CD3ε (e.g., human CD3ε) demonstrates that the test antibody can compete with any one of the presently disclosed anti-CD3 antibodies or antigen-binding fragments thereof for binding to CD3ε (e.g., human CD3E) and thus binds to the same epitope region on CD3ε (e.g., human CD3ε) as any one of the presently disclosed anti-CD3 antibodies or antigen-binding fragments thereof. In certain embodiments, the cross-competing antibody or antigen-binding fragment thereof binds to the same epitope on CD3ε (e.g., human CD3ε) as any one of the presently disclosed anti-CD3 antibodies or antigen-binding fragments thereof.

### 3.6. Characterization of Antibody Binding to Antigen

Antibodies or antigen-binding fragments thereof of the presently disclosed subject can be tested for binding to CD3ε by, for example, standard ELISA. To determine if the selected anti-CD3 antibodies bind to unique epitopes, each antibody can be biotinylated using commercially available reagents (Pierce, Rockford, IL). Competition studies using unlabeled monoclonal antibodies and biotinylated monoclonal antibodies can be performed using CD3ε coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. Anti-CD3 human IgGs can be further tested for reactivity with CD3ε antigen by Western blotting.

In certain embodiments, the K_{D} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol Biol (1999); 293:865-881).

In certain embodiments, the K_{D} is measured using a BIACORE® surface plasmon resonance assay. For example, an assay using a BIACORE®-2000 or a BIACORE®-3000 (BIAcore, Inc., Piscataway, NJ).

### 3.7. Multi-Specific Molecules

The presently disclosed subject matter provides multi-specific molecules comprising an anti-CD3 antibody, or a fragment thereof, disclosed herein. A presently disclosed or an antigen-binding fragment thereof can be derivatized or linked to one more functional molecule, e.g., one or more peptides or proteins (e.g., one or more antibodies or ligands for a receptor) to generate a multi-specific molecule that binds to two or more different binding sites or target molecules. The presently disclosed anti-CD3 antibody or antigen-binding fragment thereof can in fact be derivatized or linked to more than one other functional molecule to generate multi-specific molecules that bind to more than two different binding sites and/or target molecules. To create a multi-specific molecule, a presently disclosed anti-CD3 antibody or an antigen-binding fragment thereof can be functionally linked (e.g., by chemical coupling, genetic fusion, noncovalent association, or otherwise) to one or more other binding molecules, such as another antibody, antibody fragment, peptide or binding mimetic, such that a bispecific molecule.

In certain embodiments, the multi-specific molecule is a bispecific molecule. In certain embodiments, the bispecific molecules comprise at least a first binding specificity for CD3 and a second binding specificity for a second target epitope region. The second target epitope region can be a CD3 epitope, or a non-CD3 epitope, e.g., a different antigen. In certain embodiments, the multi-specific molecule comprises a first binding specificity for CD3, a second binding specificity for a second target, and a third binding specificity for a third target. In certain embodiments, the second target is an antigen expressed on the surface of a tumor cell. In certain embodiments, the multi-specific molecule is capable of recruiting the activity of that immune effector cell by specifically binding to the effector antigen on the human immune effector cell, thereby enhancing effector function. In certain embodiments, the third target is an antigen expressed on a senescent cell.

In certain embodiments, the bispecific molecules disclosed herein can be a fragment-based bispecific molecule. Fragment-based bispecific molecules include multiple antibody fragments and do not contain an Fc region. Non-limiting examples of fragment-based bispecific molecules include BiTE, DART, and TandAb.

In certain embodiments, the fragment-based bispecific molecule can be a bispecific T cell engager (BiTE). A BiTE comprises a scFv that binds T cell antigen (e.g., an anti-CD3 antibody, or a fragment thereof, disclosed herein) and a scFv that binds a tumor antigen (e.g., CD33) through a peptide linker. In certain embodiments, the fragment-based bispecific molecule can be a dual affinity retargeting antibody (DART). A DART comprises two separate, but paired, polypeptide chains, each comprising VL and VH regions that recognize different cell-surface molecules. In certain embodiments, the fragment-based bispecific molecule can be a tandem diabody (TandAb). A TandAb comprises two diabodies linked in a linear arrangement to produce a tetravalent bispecific molecule.

In certain embodiments, the bispecific molecules disclosed herein can be a full-length bispecific molecule. In certain embodiments, the full-length bispecific molecule includes two different antigen binding domains in a single polypeptide chain or a single set of paired light and heavy chains, while maintaining the Fc region. Multiple and different approaches can be used to generate a full-length bispecific molecule. In certain embodiments, the full-length bispecific molecule can be generated through knob-into-hole technology. The knob-into-hole technology includes the replacement of a smaller amino acid with a larger amino acid (e.g., T336Y) in the CH3 region of an antibody chain to form a “knob” structure, and at the same time substituting a larger amino acid in the other chain with a smaller amino acid to form a “hole” structure (e.g., Y407T).

In certain embodiments, the full-length bispecific molecule can be generated through SEED technology. The SEED technology uses modified sequences of IgA and IgG to produce asymmetric but complementary domains called AG and GA, which form heterodimers. In certain embodiments, the full-length bispecific molecule can be generated through DEKK technology. In DEKK, multispecific antibodies are generated by mutating a first heavy chain (e.g., amino acid substitutions L351D and L368E) and a second heavy chain (e.g., L351K and T366K) which results in their stable interaction.

In certain embodiments, the full-length bispecific molecule can be generated through Art-Ig technology. The Art-Ig technology improves heterodimerization processes by mutating the Fc in order to introduce different charges. For example, if one chain includes T394D, P395D, and P396D mutations, the other chain would include P395K, P396K, and V397K mutations. In certain embodiments, the full-length bispecific molecule can be generated through DuoBody technology. The DuoBody technology is based on a process called controlled Fab-arm exchange, a process including i) separate expression of two parental IgG1s containing single matching point mutations in the CH3 domain, ii) mixing of parental IgG1s under permissive redox conditions in vitro to enable recombination of half-molecules, and iii) removal of the reductant to allow reoxidation of interchain disulfide bonds.

In certain embodiments, the full-length bispecific molecule can be generated through DVD-Ig technology. Molecules generated using the DVD-Ig technology connect the VL and VH domains of the second antibody at the N-terminus of a first IgG antibody light chain and heavy chain, respectively, to produce a binding site with two binding sites for each antigen. In certain embodiments, the full-length bispecific molecule can be generated through CrossMab technology. CrossMab technology relies upon swapping the regions of one side heavy chain and light chain to promote correct assembly. This technology is usually combined with the knob-into-hole, DEEK, and ART-Ig technologies.

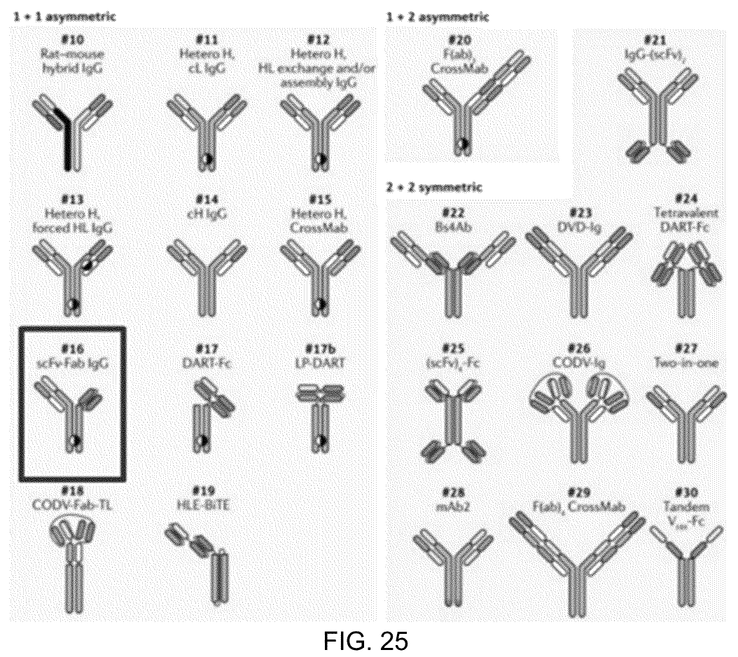

Other bispecific molecules encompassed by the presently disclosed subject matter are depicted in FIG. 25 and described in Bates and Power, Antibodies 8.2 (2019): 28; Jin et al., Signal Transduction and Targeted Therapy 7.1 (2022): 1-28; Elshiaty et al., International journal of molecular sciences 22.11 (2021): 5632; Wang et al., Antibodies 8.3 (2019): 43; Vafa and Trinklein, Frontiers in oncology 10 (2020): 446; Tian et al., Journal of Hematology & Oncology 14, no. 1 (2021): 75; and Giese et al., Biotechnology progress 34.2 (2018): 397-404, the contents of each of which is incorporated in its entirety.

The multi-specific molecules of the presently disclosed subject matter can be prepared by conjugating the constituent binding specificities using methods known in the art. For example, each binding specificity of the multi-specific molecule can be generated separately and then conjugated with one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio) propionate (SPDP), and sulfosuccinimidyl 4-(N-malcimidomethyl)cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139:2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multi-specific molecule is a mAb x mAb, mAb x Fab, Fab x F(ab′)₂ or ligand x Fab fusion protein.

Binding of the multi-specific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific to the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or autoradiography.

### 3.7.1. Antigen

In certain embodiments, the multi-specific molecule comprises at least a first binding specificity for CD3 and a second binding specificity for a second target epitope region. The second target epitope region can be a non-CD3 epitope, e.g., a different antigen. In certain embodiments, the multi-specific molecule comprises at least a first binding specificity for CD3 and a second binding specificity for a second target.

In certain embodiments, the second target is a tumor antigen. In certain embodiments, the tumor antigen is an antigen with low antigen density. In certain embodiments, the tumor antigen is expressed on a cell with low tumor cell frequency.

Any tumor antigen (antigenic peptide) can be used in the tumor-related embodiments described herein. Sources of antigen include, but are not limited to, cancer proteins. The second target can be expressed as a peptide or as an intact protein or a portion thereof. The intact protein or portion thereof can be native or mutagenized. Non-limiting examples of tumor antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD371, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGFIR, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PRI), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, U5 snRNP200, and ZDHHC11.

In certain embodiments, the second target is selected from the group consisting of CD371, U5 snRNP200, CD312, CLEC12A, CD33, CD123, IL1RAP, SIGLEC-6, GRP78, TIM3, CD70, CD20, CD22, CD19, GPRC5D, SLAMF7, BCMA, CD276, and CAIX, CD371, CD19, HVEM, LTBR, CD99, AXL, EphA2, and DLL3. In certain embodiments, the second target is CD33. In certain embodiments, the second target is CD371. In certain embodiments, the second target is U5 snRNP200.

In certain embodiments, the first antigen is a pathogen antigen. Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g. M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, Corynebacterium sp., Erysipelothrix rhusiopathiae, Clostridium spp., Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 3.7.2. Exemplary Multi-Specific Molecules

In certain embodiments, the multi-specific molecule is a bispecific molecule comprising a first binding specificity for CD3 and a second binding specificity for CD33.

In certain embodiments, the first binding specificity for CD3 comprises an anti-CD3 antibody or antigen-binding fragment thereof disclosed herein. In certain embodiments, the second binding specificity for CD33 comprises an anti-CD33 antibody or antigen-binding fragment thereof disclosed in International Patent Application No. PCT/US2022/042448, the content of which is incorporated by reference in its entirety.

In certain embodiments, the first binding specificity for CD3 comprises a VH comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a VL comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6. In certain embodiments, the second binding specificity for CD33 comprises a V_{H} comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a V_{L} comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92. SEQ ID Nos: 87-92 are provided in Table 17 below.

In certain embodiments, the bispecific molecule comprises a first binding specificity for CD3 comprising a V_{H} comprising the amino acid sequence set forth in SEQ ID NO: 7, and a V_{L} comprising the amino acid sequence set forth in SEQ ID NO: 8; and a second binding specificity for CD33 comprising a V_{H} comprising the amino acid sequence set forth in SEQ ID NO: 93, and a V_{L} comprising the amino acid sequence set forth in SEQ ID NO: 94. SEQ ID Nos: 93-94 are provided in Table 17 below

TABLE 17

| CDRs | 1 | 2 | 3 |
|---|---|---|---|
| V_{H} | GFTFSTYA | ISGRGGST | AGRGDYYYYYGMDV |
| | [SEQ ID NO: 87] | [SEQ ID NO: 88] | [SEQ ID NO: 89] |
| V_{L} | QSLVYSDGNTY | KIS | MQSTQFPHT |
| | [SEQ ID NO: 90] | [SEQ ID NO: 91] | [SEQ ID NO: 92] |

| | |
|---|---|
| Full V_{H} | EVQLLESGGGLVQPGGSLRLSCAASGFTFSTYAMSWVRQAPGKGLEWVSAISGR |
| | GGSTYYTDSVKGRFTISRDNSKNTVSLQMNSLRAEDTAVYYCAGRGDYYYYYG |
| | MDVWGQGTTVTVSA [SEQ ID NO: 93] |
| Full V_{L} | DIVMTQSPLSSPVTLGQPASFSCRSSQSLVYSDGNTYLSWLQQRPGQPPRLLIYKI |
| | SNRFSGVPDRFSGSGAGTDFTLKISRVEAEDVGVYYCMQSTQFPHTFGQGTKLEIK |
| | [SEQ ID NO: 94] |

In certain embodiments, the multi-specific molecule is a bispecific molecule comprising a first binding specificity for CD3 and a second binding specificity for CD33. In certain embodiments, the first binding specificity for CD3 comprises a V_{H} comprising a CDR1 comprising the amino acid sequence set forth in SEQ ID NO: 9, a CDR2 comprising the amino acid sequence set forth in SEQ ID NO: 10, a CDR3 comprising the amino acid sequence set forth in SEQ ID NO: 11; and a V_{L} comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14. In certain embodiments, the second binding specificity for CD33 comprises a V_{H} comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a V_{L} comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92. In certain embodiments, the bispecific molecules comprises a first binding specificity for CD3 comprising a V_{H} comprising the amino acid sequence set forth in SEQ ID NO: 15, and a V_{L} comprising the amino acid sequence set forth in SEQ ID NO: 16; and a second binding specificity for CD33 comprising a V_{H} comprising the amino acid sequence set forth in SEQ ID NO: 93, and a V_{L} comprising the amino acid sequence set forth in SEQ ID NO: 94.

In certain embodiments, the multi-specific molecule is a bispecific molecule comprising a first binding specificity for CD3 and a second binding specificity for CD33. In certain embodiments, the first binding specificity for CD3 comprises a V_{H} comprising a CDR1 comprising the amino acid sequence set forth in SEQ ID NO: 17, a CDR2 comprising the amino acid sequence set forth in SEQ ID NO: 18, a CDR3 comprising the amino acid sequence set forth in SEQ ID NO: 19; and a V_{L} comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21. In certain embodiments, the second binding specificity for CD33 comprises a V_{H} comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a V_{L} comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92. In certain embodiments, the bispecific molecules comprises a first binding specificity for CD3 comprising a V_{H} comprising the amino acid sequence set forth in SEQ ID NO: 22, and a V_{L} comprising the amino acid sequence set forth in SEQ ID NO: 23; and a second binding specificity for CD33 comprising a V_{H} comprising the amino acid sequence set forth in SEQ ID NO: 93, and a V_{L} comprising the amino acid sequence set forth in SEQ ID NO: 94.

In certain embodiments, the multi-specific molecule is a bispecific molecule comprising a first binding specificity for CD3 and a second binding specificity for CD33. In certain embodiments, the first binding specificity for CD3 comprises a V_{H} comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a V_{L} comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29. In certain embodiments, the second binding specificity for CD33 comprises a V_{H} comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a V_{L} comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92. In certain embodiments, the bispecific molecules comprises a first binding specificity for CD3 comprising a V_{H} comprising the amino acid sequence set forth in SEQ ID NO: 30, and a V_{L} comprising the amino acid sequence set forth in SEQ ID NO: 31; and a second binding specificity for CD33 comprising a V_{H} comprising the amino acid sequence set forth in SEQ ID NO: 93, and a V_{L} comprising the amino acid sequence set forth in SEQ ID NO: 94.

In certain embodiments, the multi-specific molecule is a bispecific molecule comprising a first binding specificity for CD3 and a second binding specificity for U5 snRNP200.

In certain embodiments, the first binding specificity for CD3 comprises an anti-CD3 antibody or antigen-binding fragment thereof disclosed herein. In certain embodiments, the second binding specificity for U5 snRNP200 comprises an anti-U5 snRNP200 antibody or antigen-binding fragment thereof disclosed in Gillissen et al., Blood, The Journal of the American Society of Hematology 131.1 (2018): 131-143, the content of which is incorporated by reference in its entirety.

In certain embodiments, the first binding specificity for CD3 comprises a V_{H} comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a VL comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6. In certain embodiments, the second binding specificity for U5 snRNP200 comprises a V_{H} comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. SEQ ID Nos: 116-121 are provided in Table 18 below.

In certain embodiments, the bispecific molecule comprises a first binding specificity for CD3 comprising a V_{H} comprising the amino acid sequence set forth in SEQ ID NO: 7, and a V_{L} comprising the amino acid sequence set forth in SEQ ID NO: 8; and a second binding specificity for U5 snRNP200 comprising a V_{H} comprising the amino acid sequence set forth in SEQ ID NO: 122, and a V_{L} comprising the amino acid sequence set forth in SEQ ID NO: 123. SEQ ID Nos: 122 and 123 are provided in Table 18 below

TABLE 18

| CDRs | 1 | 2 | 3 |
|---|---|---|---|
| V_{H} | GFTFSTYG | IWYDGSNT | ARGRGYSAQGNRNRA |
| | [SEQ ID NO: 116] | [SEQ ID NO: 117] | YYFDY [SEQ ID NO: |
| | | | 118] |
| V_{L} | QSVSSN | GA | QQYNDRPPYT [SEQ ID |
| | [SEQ ID NO: 119] | [SEQ ID NO: 120] | NO: 121] |

| | |
|---|---|
| Full V_{H} | QVQLVESGGGVVQPGRSLRLSCAASGFTFSTYGMHWVRQAPGKGLEWVAVIW |
| | YDGSNTYYADSVKGRFTISRDNSKNTLYLQIKSLRAEDTAVYYCARGRGYSAQ |
| | GNRNRAYYFDYWGQGTLVTVS [SEQ ID NO: 122] |
| Full V_{L} | EIVMTQSPATLSVSPGERVILSCRASQSVSSNLAWYQQKPGQPPRLLIYGAFTRV |
| | TGVPARFSGSGSGTEFTLTISSLQSEDFAVYYCQQYNDRPPYTFGQGTKLEIKRA |
| | VDQ |
| | [SEQ ID NO: 123] |
| scFv | QVQLVESGGGVVQPGRSLRLSCAASGFTFSTYGMHWVRQAPGKGLEWVAVIW |
| V_{H}-V_{L} | YDGSNTYYADSVKGRFTISRDNSKNTLYLQIKSLRAEDTAVYYCARGRGYSAQ |
| | GNRNRAYYFDYWGQGTLVTVSGGGGSGGGGSGGGGSEIVMTQSPATLSVSPGE |
| | RVILSCRASQSVSSNLAWYQQKPGQPPRLLIYGAFTRVTGVPARFSGSGSGTEFT |
| | LTISSLQSEDFAVYYCQQYNDRPPYTFGQGTKLEIKRAVDQ [SEQ ID NO: 124] |
| DNA scFv | GAGGTGCAGCTGGTGGAGTCTGGGGGAGGCGTGGTCCAGCCTGGGAGGTCC |
| V_{H}-V_{L} | CTGAGACTCTCCTGTGCAGCGTCTGGATTCACCTTCAGTACCTATGGCATGCA |
| | CTGGGTCCGCCAGGCTCCAGGCAAGGGGCTTGAGTGGGTGGCAGTTATATGG |
| | TATGATGGAAGTAATACATACTATGCAGACTCCGTGAAGGGCCGATTCACCA |
| | TCTCCAGAGACAATTCCAAGAACACACTGTATCTGCAAATAAAGAGCCTGAG |
| | AGCCGAGGACACGGCTGTCTATTACTGTGCGAGAGGCCGTGGATATAGTGCC |
| | CAAGGGAATCGGAATAGGGCTTACTACTTTGACTACTGGGGCCAGGGAACCC |
| | TGGTCACCGTCTCCGGTGGAGGTGGATCAGGTGGAGGTGGATCTGGTGGAGG |
| | TGGATCTGAAATAGTGATGACGCAGTCTCCAGCCACCCTGTCTGTGTCTCCA |
| | GGGGAAAGGGTCATCCTCTCCTGCAGGGCCAGTCAGAGTGTTAGCAGCAACT |
| | TAGCCTGGTACCAGCAGAAACCTGGCCAGCCTCCCAGGCTCCTCATCTATGG |
| | TGCATTCACGAGGGTCACTGGTGTCCCAGCCAGGTTCAGTGGCAGTGGGTCT |
| | GGGACAGAATTCACTCTCACCATCAGCAGCCTGCAGTCTGAAGATTTTGCAG |
| | TTTATTACTGTCAGCAGTACAATGACCGGCCCCCGTACACTTTTGGCCAGGG |
| | GACCAAGCTGGAGATCAAACGGGCTGTCGACCAA [SEQ ID NO: 125] |
| scFv | EIVMTQSPATLSVSPGERVILSCRASQSVSSNLAWYQQKPGQPPRLLIYGAFTRV |
| V_{L}-V_{H} | TGVPARFSGSGSGTEFTLTISSLQSEDFAVYYCQQYNDRPPYTFGQGTKLEIKRA |
| | VDQGGGGSGGGGSGGGGSQVQLVESGGGVVQPGRSLRLSCAASGFTFSTYGMH |
| | WVRQAPGKGLEWVAVIWYDGSNTYYADSVKGRFTISRDNSKNTLYLQIKSLRA |
| | EDTAVYYCARGRGYSAQGNRNRAYYFDYWGQGTLVTVS [SEQ ID NO: 126] |
| DNA scFv | GAAATAGTGATGACGCAGTCTCCAGCCACCCTGTCTGTGTCTCCAGGGGAAA |
| V_{L}-V_{H} | GGGTCATCCTCTCCTGCAGGGCCAGTCAGAGTGTTAGCAGCAACTTAGCCTG |
| | GTACCAGCAGAAACCTGGCCAGCCTCCCAGGCTCCTCATCTATGGTGCATTC |
| | ACGAGGGTCACTGGTGTCCCAGCCAGGTTCAGTGGCAGTGGGTCTGGGACAG |
| | AATTCACTCTCACCATCAGCAGCCTGCAGTCTGAAGATTTTGCAGTTTATTAC |
| | TGTCAGCAGTACAATGACCGGCCCCCGTACACTTTTGGCCAGGGGACCAAGC |
| | TGGAGATCAAACGGGCTGTCGACCAAGGAGGTGGAGGATCTGGGGGTGGAG |
| | GTTCTGGTGGGGGTGGATCTCAGGTGCAGCTGGTGGAGTCTGGGGGAGGCGT |
| | GGTCCAGCCAGGGCGGTCCCTGAGACTCTCCTGTGCAGCGTCTGGATTCACC |
| | TTCAGTACCTATGGCATGCACTGGGTCCGCCAGGCTCCAGGCAAGGGGCTTG |
| | AGTGGGTGGCAGTTATATGGTATGATGGAAGTAATACATACTATGCAGACTC |
| | CGTGAAGGGCCGATTCACCATCTCCAGAGACAATTCCAAGAACACACTGTAT |
| | CTGCAAATAAAGAGCCTGAGAGCCGAGGACACGGCTGTCTATTACTGTGCGA |
| | GAGGCCGTGGATATAGTGCCCAAGGGAATCGGAATAGGGCTTACTACTTTGA |
| | CTACTGGGGCCAGGGAACCCTGGTCACCGTCTCC [SEQ ID NO: 127] |

In certain embodiments, the multi-specific molecule is a bispecific molecule comprising a first binding specificity for CD3 and a second binding specificity for U5 snRNP200. In certain embodiments, the first binding specificity for CD3 comprises a VH comprising a CDR1 comprising the amino acid sequence set forth in SEQ ID NO: 9, a CDR2 comprising the amino acid sequence set forth in SEQ ID NO: 10, a CDR3 comprising the amino acid sequence set forth in SEQ ID NO: 11; and a VL comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14. In certain embodiments, the second binding specificity for U5 snRNP200 comprises a V_{H} comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the bispecific molecules comprises a first binding specificity for CD3 comprising a V_{H} comprising the amino acid sequence set forth in SEQ ID NO: 15, and a V_{L} comprising the amino acid sequence set forth in SEQ ID NO: 16; and a second binding specificity for U5 snRNP200 comprising a V_{H} comprising the amino acid sequence set forth in SEQ ID NO: 123, and a V_{L} comprising the amino acid sequence set forth in SEQ ID NO: 124.

In certain embodiments, the multi-specific molecule is a bispecific molecule comprising a first binding specificity for CD3 and a second binding specificity for U5 snRNP200. In certain embodiments, the first binding specificity for CD3 comprises a V_{H} comprising a CDR1 comprising the amino acid sequence set forth in SEQ ID NO: 17, a CDR2 comprising the amino acid sequence set forth in SEQ ID NO: 18, a CDR3 comprising the amino acid sequence set forth in SEQ ID NO: 19; and a V_{L} comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21. In certain embodiments, the second binding specificity for U5 snRNP200 comprises a V_{H} comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the bispecific molecules comprises a first binding specificity for CD3 comprising a V_{H} comprising the amino acid sequence set forth in SEQ ID NO: 22, and a V_{L} comprising the amino acid sequence set forth in SEQ ID NO: 23; and a second binding specificity for U5 snRNP200 comprising a V_{H} comprising the amino acid sequence set forth in SEQ ID NO: 123, and a V_{L} comprising the amino acid sequence set forth in SEQ ID NO: 124.

In certain embodiments, the multi-specific molecule is a bispecific molecule comprising a first binding specificity for CD3 and a second binding specificity for U5 snRNP200. In certain embodiments, the first binding specificity for CD3 comprises a V_{H} comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a V_{L} comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29. In certain embodiments, the second binding specificity for U5 snRNP200 comprises a V_{H} comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121. In certain embodiments, the bispecific molecules comprises a first binding specificity for CD3 comprising a V_{H} comprising the amino acid sequence set forth in SEQ ID NO: 30, and a V_{L} comprising the amino acid sequence set forth in SEQ ID NO: 31; and a second binding specificity for U5 snRNP200 comprising a V_{H} comprising the amino acid sequence set forth in SEQ ID NO: 123, and a V_{L} comprising the amino acid sequence set forth in SEQ ID NO: 124.

In certain embodiments, the multi-specific molecule is a bispecific molecule comprising a first binding specificity for CD3 and a second binding specificity for U5 snRNP200. In certain embodiments, the multi-specific molecule comprises an amino acid sequence that is at least about 80%, at least about 85%, at least about 90%, at least about 95%, at least about 96%, at least about 97%, at least about 98%, or at least about 99% identical to the amino acid sequence set forth in SEQ ID NO: 128 or a fragment thereof. In certain embodiments, the multi-specific molecule comprises or consists of an amino acid sequence identical to the amino acid sequence set forth in SEQ ID NO: 128 or a fragment thereof. SEQ ID NO: 128 is provided below:

[SEQ ID NO: 128]
MALPVTALLLPLALLLHAQVQLVESGGGVVQPGRSLRLSCAASGFTFSTYGMHWVRQAPGKGLEW
VAVIWYDGSNTYYADSVKGRFTISRDNSKNTLYLQIKSLRAEDTAVYYCARGRGYSAQGNRNRAY
YFDYWGQGTLVTVSGGGGSGGGGSGGGGSEIVMTQSPATLSVSPGERVILSCRASQSVSSNLAWYQ
QKPGQPPRLLIYGAFTRVTGVPARFSGSGSGTEFTLTISSLQSEDFAVYYCQQYNDRPPYTFGQGTKL
EIKRAVDQEQKLISEEDLAAAGGGGSQVQLVQSGAEVKKPGASVKVSCKASGYTFTAYYIHWVRQ
APGQGLEWMGWINPNSGGTNYAQMFQGRVTMTRDTSISAAYMELSRLSSDDTAVYYCTRGTPRY
NWKSYNYGMDVWGQGTTVTVSSGGGGSGGGGSGGGGSDIVVTQTPLSSPVTLGQPASISCRSSQSL
VHSDGNTYLSWLQQRPGQPPRLLIYRISNRFSGVPDRFSGSGAGTDFTLKISRVEAEDVGVYYCMQA
TQFPITFGQGTRLEIK

An exemplary nucleotide sequence encoding the amino acid sequence of SEQ ID NO: 128 is set forth in SEQ ID NO: 129, which is provided below.

[SEQ ID NO: 129]
ATGGCTCTCCCAGTGACTGCCCTACTGCTTCCCCTAGCGCTTCTCCTGCATGCACAGGTGCAGCT
GGTGGAGTCTGGGGGAGGCGTGGTCCAGCCTGGGAGGTCCCTGAGACTCTCCTGTGCAGCGTCT
GGATTCACCTTCAGTACCTATGGCATGCACTGGGTCCGCCAGGCTCCAGGCAAGGGGCTTGAGT
GGGTGGCAGTTATATGGTATGATGGAAGTAATACATACTATGCAGACTCCGTGAAGGGCCGATT
CACCATCTCCAGAGACAATTCCAAGAACACACTGTATCTGCAAATAAAGAGCCTGAGAGCCGA
GGACACGGCTGTCTATTACTGTGCGAGAGGCCGTGGATATAGTGCCCAAGGGAATCGGAATAG
GGCTTACTACTTTGACTACTGGGGCCAGGGAACCCTGGTCACCGTCTCCGGTGGAGGTGGATCA
GGTGGAGGTGGATCTGGTGGAGGTGGATCTGAAATAGTGATGACGCAGTCTCCAGCCACCCTG
TCTGTGTCTCCAGGGGAAAGGGTCATCCTCTCCTGCAGGGCCAGTCAGAGTGTTAGCAGCAACT
TAGCCTGGTACCAGCAGAAACCTGGCCAGCCTCCCAGGCTCCTCATCTATGGTGCATTCACGAG
GGTCACTGGTGTCCCAGCCAGGTTCAGTGGCAGTGGGTCTGGGACAGAATTCACTCTCACCATC
AGCAGCCTGCAGTCTGAAGATTTTGCAGTTTATTACTGTCAGCAGTACAATGACCGGCCCCCGT
ACACTTTTGGCCAGGGGACCAAGCTGGAGATCAAACGGGCTGTCGACCAAGAACAGAAACTGA
TCTCTGAAGAAGACCTGGCGGCCGCAGGTGGGGGAGGAAGTCAGGTGCAGCTGGTGCAGTCTG
GGGCTGAGGTGAAGAAGCCTGGGGCCTCAGTGAAGGTCTCCTGCAAGGCTTCTGGATACACCTT
CACCGCCTACTATATACACTGGGTGCGACAGGCCCCTGGACAAGGACTTGAGTGGATGGGATG
GATCAACCCTAACAGTGGTGGCACAAACTATGCACAGATGTTTCAGGGCAGGGTCACCATGAC
CAGGGACACGTCCATCAGCGCAGCCTACATGGAGCTGAGCAGGCTGAGCTCTGACGACACGGC
CGTATATTACTGTACGAGAGGAACTCCCCGTTATAACTGGAAGTCCTACAACTACGGTATGGAC
GTCTGGGGCCAAGGGACCACGGTCACCGTCTCCTCAGGAGGGGGTGGTTCTGGAGGTGGTGGG
TCAGGGGGGGGGGGCTCCGATATTGTGGTGACCCAGACTCCACTCTCCTCACCTGTCACTCTTG
GACAACCGGCCTCCATCTCCTGCAGGTCTAGTCAAAGCCTCGTACACAGTGATGGAAACACCTA
CTTGAGTTGGCTTCAGCAGAGGCCAGGCCAGCCTCCAAGACTCCTAATTTATAGGATTTCTAAC
CGTTTCTCTGGGGTCCCAGACAGATTCAGTGGCAGTGGGGCTGGGACAGATTTCACACTGAAGA
TCAGCAGGGTGGAAGCTGAGGATGTCGGGGTTTATTATTGCATGCAAGCTACACAATTTCCTAT
CACCTTCGGCCAAGGGACACGACTGGAGATTAAA

In certain embodiments, the multi-specific molecule comprises an amino acid sequence that is at least about 80%, at least about 85%, at least about 90%, at least about 95%, at least about 96%, at least about 97%, at least about 98%, or at least about 99% identical to the amino acid sequence set forth in SEQ ID NO: 130 or a fragment thereof. In certain embodiments, the multi-specific molecule comprises or consists of an amino acid sequence identical to the amino acid sequence set forth in SEQ ID NO: 130 or a fragment thereof. SEQ ID NO: 130 is provided below:

[SEQ ID NO: 130]
MALPVTALLLPLALLLHAEIVMTQSPATLSVSPGERVILSCRASQSVSSNLAWYQQKPGQPPRLLIYG
AFTRVTGVPARFSGSGSGTEFTLTISSLQSEDFAVYYCQQYNDRPPYTFGQGTKLEIKRAVDQGGGG
SGGGGSGGGGSQVQLVESGGGVVQPGRSLRLSCAASGFTFSTYGMHWVRQAPGKGLEWVAVIWY
DGSNTYYADSVKGRFTISRDNSKNTLYLQIKSLRAEDTAVYYCARGRGYSAQGNRNRAYYFDYWG
QGTLVTVSEQKLISEEDLAAAGGGGSQVQLVQSGAEVKKPGASVKVSCKASGYTFTAYYIHWVRQ
APGQGLEWMGWINPNSGGTNYAQMFQGRVTMTRDTSISAAYMELSRLSSDDTAVYYCTRGTPRY
NWKSYNYGMDVWGQGTTVTVSSGGGGSGGGGSGGGGSDIVVTQTPLSSPVTLGQPASISCRSSQSL
VHSDGNTYLSWLQQRPGQPPRLLIYRISNRFSGVPDRFSGSGAGTDFTLKISRVEAEDVGVYYCMQA
TQFPITFGQGTRLEIK

An exemplary nucleotide sequence encoding the amino acid sequence of SEQ ID NO: 130 is set forth in SEQ ID NO: 131, which is provided below.

[SEQ ID NO: 131]
ATGGCTCTCCCAGTGACTGCCCTACTGCTTCCCCTAGCGCTTCTCCTGCATGCAgaaatagtgatgacgca
gtctccagccaccctgtctgtgtctccaggggaaagggtcatcctctcctgcagggccagtcagagtgttagcagcaacttagcctggtacc
agcagaaacctggccagcctcccaggctcctcatctatggtgcattcacgagggtcactggtgtcccagccaggttcagtggcagtgggtct
gggacagaattcactctcaccatcagcagcctgcagtctgaagattttgcagtttattactgtcagcagtacaatgaccggcccccgtacac
ttttggccaggggaccaagctggagatcaaaCGGGCTGTCGACCAAGGAGGTGGAGGATCTGGGGGTGGAGGTTCTGGTGGGGGTGGATCTc
aggtgcagctggtggagtctgggggaggcgtggtccagccagggcggtccctgagactctcctgtgcagcgtctggattcaccttcagtacc
tatggcatgcactgggtccgccaggctccaggcaaggggcttgagtgggtggcagttatatggtatgatggaagtaatacatactatgcaga
ctccgtgaagggccgattcaccatctccagagacaattccaagaacacactgtatctgcaaataaagagcctgagagccgaggacacggctg
tctattactgtgcgagaggccgtggatatagtgcccaagggaatcggaatagggcttactactttgactactggggccagggaaccctggtc
accgtctccGAACAGAAACTGATCTCTGAAGAAGACCTGGCGGCCGCAGGTGGGGGAGGAAGTCAGGTGCAGCTGGTGCAGTCTGGGGCTGA
GGTGAAGAAGCCTGGGGCCTCAGTGAAGGTCTCCTGCAAGGCTTCTGGATACACCTTCACCGCCTACTA
TATACACTGGGTGCGACAGGCCCCTGGACAAGGACTTGAGTGGATGGGATGGATCAACCCTAA
CAGTGGTGGCACAAACTATGCACAGATGTTTCAGGGCAGGGTCACCATGACCAGGGACACGTC
CATCAGCGCAGCCTACATGGAGCTGAGCAGGCTGAGCTCTGACGACACGGCCGTATATTACTGT
ACGAGAGGAACTCCCCGTTATAACTGGAAGTCCTACAACTACGGTATGGACGTCTGGGGCCAA
GGGACCACGGTCACCGTCTCCTCAGGAGGGGGTGGTTCTGGAGGTGGTGGGTCAGGGGGGGGG
GGCTCCGATATTGTGGTGACCCAGACTCCACTCTCCTCACCTGTCACTCTTGGACAACCGGCCTC
CATCTCCTGCAGGTCTAGTCAAAGCCTCGTACACAGTGATGGAAACACCTACTTGAGTTGGCTT
CAGCAGAGGCCAGGCCAGCCTCCAAGACTCCTAATTTATAGGATTTCTAACCGTTTCTCTGGGG
TCCCAGACAGATTCAGTGGCAGTGGGGCTGGGACAGATTTCACACTGAAGATCAGCAGGGTGG
AAGCTGAGGATGTCGGGGTTTATTATTGCATGCAAGCTACACAATTTCCTATCACCTTCGGCCA
AGGGACACGACTGGAGATTAAA

In certain embodiments, the multi-specific molecule is a bispecific molecule comprising a first binding specificity for CD3 and a second binding specificity for CD371.

In certain embodiments, the first binding specificity for CD3 comprises an anti-CD3 antibody or antigen-binding fragment thereof disclosed herein. In certain embodiments, the second binding specificity for CD371 comprises an anti-CD371 antibody or antigen-binding fragment thereof disclosed in International Patent Publication No. WO 2021/050857, the content of which is incorporated by reference in its entirety.

In certain embodiments, the first binding specificity for CD3 comprises a VH comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6. In certain embodiments, the second binding specificity for CD371 comprises a V_{H} comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134; and a V_{L} comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137. SEQ ID Nos: 132-137 are provided in Table 18 below.

In certain embodiments, the bispecific molecule comprises a first binding specificity for CD3 comprising a V_{H} comprising the amino acid sequence set forth in SEQ ID NO: 7, and a V_{L} comprising the amino acid sequence set forth in SEQ ID NO: 8; and a second binding specificity for CD371 comprising a V_{H} comprising the amino acid sequence set forth in SEQ ID NO: 140, and a V_{L} comprising the amino acid sequence set forth in SEQ ID NO: 142. SEQ ID Nos: 140 and 142 are provided in Table 19 below

TABLE 19

| CDRs | 1 | 2 | 3 |
|---|---|---|---|
| V_{H} | GFTFSDYQ | IQGGGGST [SEQ ID NO: | AREMWRGDYYSGMDV |
| | [SEQ ID NO: 132] | 133] | [SEQ ID NO: 134] |
| V_{L} | QSVLDSYNNENN [SEQ | WAS [SEQ ID NO: 136] | QQYTSEPIT [SEQ ID |
| | ID NO: 135] | | NO: 137] |

| | |
|---|---|
| Full V_{H} | EVQLLESGGGLVQPGGSLRLSCAASGFTFSDYQMSWVRQAPGKGLEWVSGIQG |
| | GGGSTYYADSVKGRFTISRDNSKNTLYLQMNSLRAEDTAVYYCAREMWRGDY |
| | YSGMDVWGQGTTVTVSS [SEQ ID NO: 138] |
| Full V_{L} | DIVMTQSPDSLAVSLGERATINCKSSQSVLDSYNNENNLAWYQQKPGQPPKLLI |
| | YWASTRESGVPDRFSGSGSGTDFTLTISSLQAEDVAVYYCQQYTSEPITFGQGTK |
| | VEIK |
| | [SEQ ID NO: 139] |
| scFv | EVQLLESGGGLVQPGGSLRLSCAASGFTFSDYQMSWVRQAPGKGLEWVSGIQG |
| V_{H}-V_{L} | GGGSTYYADSVKGRFTISRDNSKNTLYLQMNSLRAEDTAVYYCAREMWRGDY |
| | YSGMDVWGQGTTVTVSSGGGGSGGGGSGGGGSGGGSGGGGSDIVMTQSPDSL |
| | AVSLGERATINCKSSQSVLDSYNNENNLAWYQQKPGQPPKLLIYWASTRESGVP |
| | DRFSGSGSGTDFTLTISSLQAEDVAVYYCQQYTSEPITFGQGTKVEIK [SEQ ID |
| | NO: 140] |
| DNA scFv | GAGGTGCAGCTGTTGGAGTCTGGGGGAGGCTTGGTACAGCCTGGGGGGTCCC |
| V_{H}-V_{L} | TGCGACTCTCCTGTGCAGCCTCTGGATTCACCTTTAGCGACTATCAGATGAGC |
| | TGGGTCCGCCAGGCTCCAGGGAAGGGGCTGGAGTGGGTGTCAGGCATTCAG |
| | GGTGGCGGTGGTAGCACATATTACGCAGACTCCGTGAAGGGCCGGTTCACCA |
| | TCTCCCGTGACAATTCCAAGAACACGCTGTATCTGCAAATGAACAGCCTGCG |
| | TGCCGAGGACACGGCTGTGTATTACTGTGCGAGAGAGATGTGGCGTGGGGA |
| | CTACTACTCCGGTATGGACGTCTGGGGCCAGGGGACCACGGTCACCGTCTCC |
| | TCAGGTGGTGGTGGTTCAGGTGGTGGTGGTTCTGGAGGGGGCGGTTCTGGCG |
| | GCGGCTCCGGTGGTGGTGGATCCGACATCGTGATGACCCAGTCTCCAGACTC |
| | CCTGGCTGTGTCTCTGGGCGAGCGTGCCACCATCAACTGCAAGTCCAGCCAG |
| | AGTGTTTTAGACAGCTATAACAATGAGAACAATTTAGCTTGGTATCAGCAGA |
| | AACCAGGACAGCCTCCTAAGCTGCTCATTTACTGGGCATCTACCCGGGAATC |
| | CGGGGTCCCTGACCGATTCAGTGGCAGCGGGTCTGGGACAGATTTCACTCTC |
| | ACCATCAGCAGCCTGCAGGCTGAAGATGTGGCAGTTTATTACTGTCAGCAAT |
| | ATACCAGCGAACCTATCACGTTCGGCCAAGGTACCAAGGTGGAAATCAAA |
| | [SEQ ID NO: 141] |
| scFv | DIVMTQSPDSLAVSLGERATINCKSSQSVLDSYNNENNLAWYQQKPGQPPKLLI |
| V_{L}-V_{H} | YWASTRESGVPDRFSGSGSGTDFTLTISSLQAEDVAVYYCQQYTSEPITFGQGTK |
| | VEIKGGGGSGGGGSGGGSGGGGSEVQLLESGGGLVQPGGSLRLSCAASGFTFSD |
| | YQMSWVRQAPGKGLEWVSGIQGGGGSTYYADSVKGRFTISRDNSKNTLYLQM |
| | NSLRAEDTAVYYCAREMWRGDYYSGMDVWGQGTTVTVSS [SEQ ID NO: 142] |
| DNA scFv | GACATCGTGATGACCCAGTCTCCAGACTCCCTGGCTGTGTCTCTGGGCGAGC |
| V_{L}-V_{H} | GTGCCACCATCAACTGCAAGTCCAGCCAGAGTGTTTTAGACAGCTATAACAA |
| | TGAGAACAATTTAGCTTGGTATCAGCAGAAACCAGGACAGCCTCCTAAGCTG |
| | CTCATTTACTGGGCATCTACCCGGGAATCCGGGGTCCCTGACCGATTCAGTG |
| | GCAGCGGGTCTGGGACAGATTTCACTCTCACCATCAGCAGCCTGCAGGCTGA |
| | AGATGTGGCAGTTTATTACTGTCAGCAATATACCAGCGAACCTATCACGTTC |
| | GGCCAAGGTACCAAGGTGGAAATCAAAGGTGGTGGTGGTTCAGGTGGTGGT |
| | GGTTCTGGCGGCGGCTCCGGTGGTGGTGGATCCGAGGTGCAGCTGTTGGAGT |
| | CTGGGGGAGGCTTGGTACAGCCTGGGGGGTCCCTGCGACTCTCCTGTGCAGC |
| | CTCTGGATTCACCTTTAGCGACTATCAGATGAGCTGGGTCCGCCAGGCTCCA |
| | GGGAAGGGGCTGGAGTGGGTGTCAGGCATTCAGGGTGGCGGTGGTAGCACA |
| | TATTACGCAGACTCCGTGAAGGGCCGGTTCACCATCTCCCGTGACAATTCCA |
| | AGAACACGCTGTATCTGCAAATGAACAGCCTGCGTGCCGAGGACACGGCTGT |
| | GTATTACTGTGCGAGAGAGATGTGGCGTGGGGACTACTACTCCGGTATGGAC |
| | GTCTGGGGCCAGGGGACCACGGTCACCGTCTCCTCA [SEQ ID NO: 143] |

In certain embodiments, the multi-specific molecule is a bispecific molecule comprising a first binding specificity for CD3 and a second binding specificity for CD371. In certain embodiments, the first binding specificity for CD3 comprises a VH comprising a CDR1 comprising the amino acid sequence set forth in SEQ ID NO: 9, a CDR2 comprising the amino acid sequence set forth in SEQ ID NO: 10, a CDR3 comprising the amino acid sequence set forth in SEQ ID NO: 11; and a VL comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14. In certain embodiments, the second binding specificity for CD371 comprises a V_{H} comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134; and a V_{L} comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137. In certain embodiments, the bispecific molecules comprises a first binding specificity for CD3 comprising a V_{H} comprising the amino acid sequence set forth in SEQ ID NO: 15, and a V_{L} comprising the amino acid sequence set forth in SEQ ID NO: 16; and a second binding specificity for CD371comprising a V_{H} comprising the amino acid sequence set forth in SEQ ID NO: 140, and a V_{L} comprising the amino acid sequence set forth in SEQ ID NO: 142.

In certain embodiments, the multi-specific molecule is a bispecific molecule comprising a first binding specificity for CD3 and a second binding specificity for CD371. In certain embodiments, the first binding specificity for CD3 comprises a V_{H} comprising a CDR1 comprising the amino acid sequence set forth in SEQ ID NO: 17, a CDR2 comprising the amino acid sequence set forth in SEQ ID NO: 18, a CDR3 comprising the amino acid sequence set forth in SEQ ID NO: 19; and a V_{L} comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21. In certain embodiments, the second binding specificity for CD371 comprises a V_{H} comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134; and a V_{L} comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137. In certain embodiments, the bispecific molecules comprises a first binding specificity for CD3 comprising a V_{H} comprising the amino acid sequence set forth in SEQ ID NO: 22, and a V_{L} comprising the amino acid sequence set forth in SEQ ID NO: 23; and a second binding specificity for CD371comprising a V_{H} comprising the amino acid sequence set forth in SEQ ID NO: 140, and a V_{L} comprising the amino acid sequence set forth in SEQ ID NO: 142.

In certain embodiments, the multi-specific molecule is a bispecific molecule comprising a first binding specificity for CD3 and a second binding specificity for CD371. In certain embodiments, the first binding specificity for CD3 comprises a VH comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a VL comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29. In certain embodiments, the second binding specificity for CD371 comprises a V_{H} comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134; and a V_{L} comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137. In certain embodiments, the bispecific molecules comprises a first binding specificity for CD3 comprising a V_{H} comprising the amino acid sequence set forth in SEQ ID NO: 30, and a V_{L} comprising the amino acid sequence set forth in SEQ ID NO: 31; and a second binding specificity for CD371 comprising a V_{H} comprising the amino acid sequence set forth in SEQ ID NO: 140, and a V_{L} comprising the amino acid sequence set forth in SEQ ID NO: 142.

In certain embodiments, the multi-specific molecule is a bispecific molecule comprising a first binding specificity for CD3 and a second binding specificity for CD371. In certain embodiments, the multi-specific molecule comprises an amino acid sequence that is at least about 80%, at least about 85%, at least about 90%, at least about 95%, at least about 96%, at least about 97%, at least about 98%, or at least about 99% identical to the amino acid sequence set forth in SEQ ID NO: 144 or a fragment thereof. In certain embodiments, the multi-specific molecule comprises or consists of an amino acid sequence identical to the amino acid sequence set forth in SEQ ID NO: 144 or a fragment thereof. SEQ ID NO: 144 is provided below:

[SEQ ID NO: 144]
MALPVTALLLPLALLLHAEVQLLESGGGLVQPGGSLRLSCAASGFTFSDYQMSWVRQAPGKGLEW
VSGIQGGGGSTYYADSVKGRFTISRDNSKNTLYLQMNSLRAEDTAVYYCAREMWRGDYYSGMDV
WGQGTTVTVSSGGGGSGGGGSGGGGSGGGSGGGGSDIVMTQSPDSLAVSLGERATINCKSSQSVLD
SYNNENNLAWYQQKPGQPPKLLIYWASTRESGVPDRFSGSGSGTDFTLTISSLQAEDVAVYYCQQY
TSEPITFGQGTKVEIKEQKLISEEDLAAAGGGGSQVQLVQSGAEVKKPGASVKVSCKASGYTFTAYY
IHWVRQAPGQGLEWMGWINPNSGGTNYAQMFQGRVTMTRDTSISAAYMELSRLSSDDTAVYYCT
RGTPRYNWKSYNYGMDVWGQGTTVTVSSGGGGSGGGGSGGGGSDIVVTQTPLSSPVTLGQPASIS
CRSSQSLVHSDGNTYLSWLQQRPGQPPRLLIYRISNRFSGVPDRFSGSGAGTDFTLKISRVEAEDVGV
YYCMQATQFPITFGQGTRLEIK

An exemplary nucleotide sequence encoding the amino acid sequence of SEQ ID NO: 144 is set forth in SEQ ID NO: 145, which is provided below.

[SEQ ID NO: 145]
ATGGCTCTCCCAGTGACTGCCCTACTGCTTCCCCTAGCGCTTCTCCTGCATGCAGAGGTGCAGCT
GTTGGAGTCTGGGGGAGGCTTGGTACAGCCTGGGGGGTCCCTGCGACTCTCCTGTGCAGCCTCT
GGATTCACCTTTAGCGACTATCAGATGAGCTGGGTCCGCCAGGCTCCAGGGAAGGGGCTGGAG
TGGGTGTCAGGCATTCAGGGTGGCGGTGGTAGCACATATTACGCAGACTCCGTGAAGGGCCGG
TTCACCATCTCCCGTGACAATTCCAAGAACACGCTGTATCTGCAAATGAACAGCCTGCGTGCCG
AGGACACGGCTGTGTATTACTGTGCGAGAGAGATGTGGCGTGGGGACTACTACTCCGGTATGG
ACGTCTGGGGCCAGGGGACCACGGTCACCGTCTCCTCAGGTGGTGGTGGTTCAGGTGGTGGTGG
TTCTGGAGGGGGCGGTTCTGGCGGCGGCTCCGGTGGTGGTGGATCCGACATCGTGATGACCCAG
TCTCCAGACTCCCTGGCTGTGTCTCTGGGCGAGCGTGCCACCATCAACTGCAAGTCCAGCCAGA
GTGTTTTAGACAGCTATAACAATGAGAACAATTTAGCTTGGTATCAGCAGAAACCAGGACAGC
CTCCTAAGCTGCTCATTTACTGGGCATCTACCCGGGAATCCGGGGTCCCTGACCGATTCAGTGG
CAGCGGGTCTGGGACAGATTTCACTCTCACCATCAGCAGCCTGCAGGCTGAAGATGTGGCAGTT
TATTACTGTCAGCAATATACCAGCGAACCTATCACGTTCGGCCAAGGTACCAAGGTGGAAATCA
AAGAACAGAAACTGATCTCTGAAGAAGACCTGGCGGCCGCAGGTGGGGGAGGAAGTCAGGTG
CAGCTGGTGCAGTCTGGGGCTGAGGTGAAGAAGCCTGGGGCCTCAGTGAAGGTCTCCTGCAAG
GCTTCTGGATACACCTTCACCGCCTACTATATACACTGGGTGCGACAGGCCCCTGGACAAGGAC
TTGAGTGGATGGGATGGATCAACCCTAACAGTGGTGGCACAAACTATGCACAGATGTTTCAGG
GCAGGGTCACCATGACCAGGGACACGTCCATCAGCGCAGCCTACATGGAGCTGAGCAGGCTGA
GCTCTGACGACACGGCCGTATATTACTGTACGAGAGGAACTCCCCGTTATAACTGGAAGTCCTA
CAACTACGGTATGGACGTCTGGGGCCAAGGGACCACGGTCACCGTCTCCTCAGGAGGGGGTGG
TTCTGGAGGTGGTGGGTCAGGGGGGGGGGGGTCCGATATTGTGGTGACCCAGACTCCACTCTCC
TCACCTGTCACTCTTGGACAACCGGCCTCCATCTCCTGCAGGTCTAGTCAAAGCCTCGTACACA
GTGATGGAAACACCTACTTGAGTTGGCTTCAGCAGAGGCCAGGCCAGCCTCCAAGACTCCTAAT
TTATAGGATTTCTAACCGTTTCTCTGGGGTCCCAGACAGATTCAGTGGCAGTGGGGCTGGGACA
GATTTCACACTGAAGATCAGCAGGGTGGAAGCTGAGGATGTCGGGGTTTATTATTGCATGCAAG
CTACACAATTTCCTATCACCTTCGGCCAAGGGACACGACTGGAGATTAAA

### 3.8. Antibody Fragments

The presently disclosed subject matter provides antibody fragments of an anti-CD3 antibody disclosed herein. In certain embodiments, the antibody fragment comprises an scFv as disclosed herein in Section 3.1. In certain embodiments, the antibody fragment is an scFv as disclosed herein in Section 3.1. In certain embodiments, the antibody fragment is a Fab fragment, a Fab′ fragment, a Fab′-SH fragment, or a F(ab′)₂ fragment.

In certain embodiments, the antibody fragment is a “Fab” fragments. “Fab” fragments can be produced by papain digestion of full length antibodies. Traditionally, Fab fragments contain the heavy-chain variable domain (V_{H}) and light-chain variable domain (V_{H}) and also the constant domain of the light chain (CL) and the first constant domain of the heavy chain (CH1).

In certain embodiments, the antibody fragment is a “Fab′” fragments. Fab′ fragments can be distinguished from Fab fragments because including additional residues at the carboxy terminus of the CH1 domain. In certain embodiments, the Fab′ fragments include one or more cysteines from the antibody hinge region.

In certain embodiments, the antibody fragment is a “Fab′-SH” fragments. Fab′-SH are Fab′ fragments where at least one cysteine residue of the constant domains includes a free thiol group.

In certain embodiments, the antibody fragment is a “F(ab′)₂” fragment. F(ab′)₂ fragments can be obtained by pepsin digestion of full length antibodies. F(ab′)₂ fragments have two antigen-binding sites (e.g., two Fab fragments) and a portion of the Fc region.

In certain embodiments, the antibody fragment is a single-domain antibody. Single-domain antibody are antibody fragments including the heavy chain variable domain or a portion thereof of an antibody or the light chain variable domain or a portion thereof of an antibody.

### 3.9. Humanized or Chimeric Antibodies

The presently disclosed subject matter further provides chimeric and/or humanized versions of an anti-CD3 antibody, or a fragment thereof, disclosed herein.

In certain embodiments, the anti-CD3 antibody is a chimeric antibody. In certain embodiments, the chimeric antibody comprises a variable region derived from a non-human species (e.g., a variable region derived from a mouse, a rat, a hamster, a rabbit, or a non-human primate) and a human constant region. Alternatively or additionally, a chimeric antibody can be a “class-switched” antibody. In certain embodiments, the class-switched antibody is an antibody wherein the class or subclass has been modified from that of the parent antibody.

In certain embodiments, the anti-CD3 antibody provided herein is a humanized antibody. In certain embodiments, the humanized antibody comprises at least one variable domain. In certain embodiments, the variable domain comprises CDRs derived from a non-human antibody, e.g., a mouse antibody.

In certain embodiments, the variable domain comprises framework regions (FR) derived from human antibody sequences. In certain embodiments, the FR include substitutions and/or modification. In certain embodiments, residues in a humanized antibody can be substituted with corresponding residues from a non-human antibody (e.g., the antibody from which the CDR residues are derived), to restore or improve antibody specificity or affinity.

In certain embodiments, the humanized antibody can also include a human constant region. In certain embodiments, humanization of a non-human antibody, e.g. a mouse antibody, reduces immunogenicity of the antibody in humans. In certain embodiments, humanization of a non-human antibody, e.g. a mouse antibody, does not impair the specificity and affinity of the parental non-human antibody.

### 3.10. Immunoconjugates

The presently disclosed subject provides an anti-CD3 antibody or an antigen-binding fragment thereof, conjugated to a therapeutic moiety, such as a cytotoxin, a drug (e.g., an immunosuppressant) or a radiotoxin. Such conjugates are referred to herein as “immunoconjugates.” Immunoconjugates that include one or more cytotoxins are referred to as “immunotoxins.” A cytotoxin or cytotoxic agent includes any agent that is detrimental to (e.g., kills) cells. Non-limiting Examples of cytotoxins include taxol (such as ricin, diphtheria, gelonin), cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, calecheamicin, aureastatin, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustinc (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), hypomethylating agents (azacytidine and decitabine), and anti-mitotic agents (e.g., vincristine and vinblastine).

Other examples of therapeutic cytotoxins that can be conjugated to an anti-CD3 antibody disclosed herein include duocarmycins, calicheamicins, maytansines and auristatins, and derivatives thereof. Cytotoxins can be conjugated to an anti-CD3 antibody or an antigen-binding fragment thereof disclosed herein using linker technology available in the art. Examples of linker types that have been used to conjugate a cytotoxin to an antibody include, but are not limited to, hydrazones, thioethers, esters, disulfides, and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Anti-CD3 antibodies or antigen-binding fragments thereof of the presently disclosed subject matter also can be conjugated to a radioactive isotope to generate cytotoxic radiopharmaceuticals, also referred to as radioimmunoconjugates. Non-limiting examples of radioactive isotopes that can be conjugated to antibodies for use diagnostically or therapeutically include ⁴⁷Sc, ⁶⁷Cu, ⁹⁰Y, ¹³¹I, ¹⁴⁹Tb, ¹⁶¹Tb, ¹⁷⁷Lu, ²²⁵Ac, ²¹³Bi, ²²³Ra and ²²⁷Th. Methods for preparing radioimmunconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the presently disclosed anti-CD3 antibodies.

In certain embodiments, the anti-CD3 antibodies or antigen-binding fragments thereof of the presently disclosed subject matter can be conjugated to a radioisotope to generate a radioimmunoconjugate by using a chelator. As used herein, the term “chelator” refers to a chemical compound in the form of a heterocyclic ring or surrounding structure containing a metal ion attached by coordinate bonds to at least two nonmetal ions. Non-limiting examples of chelator include 1,4,7-Triazacyclononane-1,4,7-triacetic acid (NOTA), 2,2′-(7-(1-carboxy-4-((4-isothiocyanatobenzyl)amino)-4-oxobutyl)-1,4,7-triazonane-1,4-diyl)diacetic acid (NODA), 2,2′,2″,2″-(1,4,7,10-Tetraazacyclododecane-1,4,7,10-tetrayl) tetraacetic acid (DOTA), Diethylenetriamine-N,N,N′,N,N-pentaacetic acid, pentetic acid, (Carboxymethyl)imino]bis(ethylenenitrilo)-tetra-acetic acid (DTPA), 1-Hydroxy-2-pyridone; 2-Pyridinol-1-oxide (HOPO), N-(5-(3-((5-Aminopentyl) hydroxycarbamoyl) propionamido) pentyl)-3-((5-(N-hydroxyacctamido) pentyl) carbamoyl) propionohydroxamic acid (DFO), and 2-[1,4,7-Triazacyclononan-1-yl-4,7-bis(tBu-ester)]-1,5-pentanedioic acid (NODAGA). Additional exemplary chelators encompassed by the presently disclosed subject matter include AAZTA and derivatives thereof, BAT, BARAC, BPCA, TE2A, CB-TE2A, CBOTEIAIP, CB-TE2P, MM-TE2A, DM TE-2A, CP356, DATA, DBCO, DiAmSar and derivatives thereof, DIBO, DIMA, DFO, DGO, DOTA and derivatives thereof (e.g., Ac-DOTA, benzo-DOTA, dibenzo-DOTA, CB-DO2A, 3p-C-DEPA, Oxo-DO3A), DOTMA derivative thereof (e.g., benzo-DOTMA), DTPA and derivatives thereof (e.g., benzo-DTPA, dibenzo-DTPA, phenyl-DTPA, diphenyl-DTPA, benzyl-DTPA, dibenzyl-DTPA, 1B4M-DTPA, CHX-A″-DTPA), EDTA, EGTA, EHPG and derivatives thereof (e.g., 5-Cl-EHPG, 5-Br-EHPG, 5-Me-EHPG, 5t-Bu-EHPG, 5-scc-Bu-EHPG), H2dedpa, H4octapa, H2azapa, H5decapa, H6phospa, HBED and derivatives thereof, SHBED, HEHA, HYNIC, LICAM and derivatives thereof, MECAM, NODASA, NODAGA, NOPO, NOTA and derivatives thereof (e.g., benzo-NOTA), NETA, PEPA, PCTA, PDTA, TACN-TM, TCMC, TETA and derivatives thereof (e.g., benzo-TETA), TETMA and derivatives (e.g., benzo-TETMA), TRAP (PRP9), TRITA, TTHA and derivatives thereof. The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor (TNF) or interferon-y; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

In certain embodiments, anti-CD3 antibodies or antigen-binding fragments thereof of the presently disclosed subject matter can be conjugated to non-radioactive isotopes that can be irradiated, e.g., with neutrons, to undergo a decay reaction and generate alpha particles. For example, but not by way of limitation, an anti-CD3 antibodies or antigen-binding fragments thereof of the presently disclosed subject matter also can be conjugated to a molecule comprising boron (e.g., 10B), a stable non-radioactive isotope of boron. The presently disclosed conjugates comprising boron can be used in boron neutron capture therapy (BNCT). As used herein, “boron neutron capture therapy” or “BNCT” refers to a targeted radiotherapy, wherein boron of the presently disclosed immunoconjugates comprising boron are irradiated with low energy thermal neutrons to yield biologically destructive alpha particles and lithium-7 nuclei. Additional information concerning immunoconjugates comprising boron and BNCT can be found in Guan et al., Proceedings of the National Academy of Sciences 95, no. 22 (1998): 13206-13210; Barth et al., Cancer Communications 38, no. 1 (2018): 1-15; Nedunchezhian et al., Journal of clinical and diagnostic research: JCDR 10, no. 12 (2016): ZE01; Malouff et al., Frontiers in oncology 11 (2021): 601820; and U.S. Pat. No. 4,624,846; the content of each of which is incorporated by reference in its entirety.

Anti-CD3 antibodies or antigen-binding fragments thereof of the presently disclosed subject matter also can be conjugated to a self-assembly disassembly (SADA) polypeptide. A SADA domain is composed of multimerization domains which are each composed of helical bundles that associate in a parallel or anti-parallel orientation. Non-limiting examples of SADA domain containing human polypeptides include p53, p63, p73, heterogeneous nuclear Ribonucleoprotein C (hnRNPC), C or N-terminal domain of Synaptosomal-associated protein 23 (SNAP-23), Cyclin-D-related protein (CBFA2T1), variants thereof, or fragments thereof. In certain embodiments, these conjugates can multimerize to form a complex of a desired size under relevant conditions (e.g., in a solution in which the conjugate is present above a threshold concentration or pH and/or when present at a target site characterized by a relevant level or density of receptors for the payload), and disassemble to a smaller form under other conditions (e.g., absent the relevant environmental multimerization trigger). Additional information concerning the SADA polypeptides and immunoconjugates comprising the same can be found in International Patent Publication No. WO 2018/204873, the content of which is incorporated by reference in its entirety.

The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor (TNF) or interferon-y; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### 3.11. Immunocytokines

Anti-CD3 antibodies or antigen-binding fragments thereof of the presently disclosed subject matter also can be conjugated to a cytokine to generate an immunocytokine. As used herein, the term “immunocytokine” refers to fusion proteins that combine antigen-binding capabilities of an antibody (e.g., an anti-CD3 antibody or antigen-fragment thereof disclosed herein) and the activity of a cytokine (e.g., IL33, IL36, or IL18). Additional information concerning immunocytokines can be found in Runbeck et al., Antibodies 10.1 (2021): 10, the content of which is incorporated by reference in its entirety.

In certain embodiments, the immunocytokine comprises an anti-CD3 antibody or an antigen-binding fragment thereof disclosed herein and an IL33 polypeptide. In certain embodiments, the IL33 polypeptide comprises or consists of an amino acid sequence that is at least about 80%, at least about 85%, at least about 90%, at least about 95%, at least about 96%, at least about 97%, at least about 98%, or at least about 99% identical to the amino acid sequence set forth in SEQ ID NO: 73 or a fragment thereof. In certain embodiments, the IL33 polypeptide comprises or consists of an amino acid sequence identical to the amino acid sequence set forth in SEQ ID NO: 73 or a fragment thereof.

[SEQ ID NO: 73]
MKPKMKYSTNKISTAKWKNTASKALCFKLGKSQQKAKEVCPMYFMKLRS
GLMIKKEACYFRRETTKRPSLKTGRKHKRHLVLAACQQQSTVECFAFGI
SGVQKYTRALHDSSITGISPITEYLASLSTYNDQSITFALEDESYEIYV
EDLKKDEKKDKVLLSYYESQHPSNESGDGVDGKMLMVTLSPTKDFWLHA
NNKEHSVELHKCEKPLPDQAFFVLHNMHSNCVSFECKTDPGVFIGVKDN
HLALIKVDSSENLCTENILFKLSET

In certain embodiments, the immunocytokine comprises an anti-CD3 antibody or an antigen-binding fragment thereof disclosed herein and an IL36 polypeptide. In certain embodiments, the IL36 polypeptide comprises or consists of an amino acid sequence that is at least about 80%, at least about 85%, at least about 90%, at least about 95%, at least about 96%, at least about 97%, at least about 98%, or at least about 99% identical to the amino acid sequence set forth in SEQ ID NO: 72 or a fragment thereof. In certain embodiments, the IL36 polypeptide comprises or consists of an amino acid sequence identical to the amino acid sequence set forth in SEQ ID NO: 72 or a fragment thereof.

[SEQ ID NO: 72]
MRGTPGDADGGGRAVYQSMCKPITGTINDLNQQVWTLQGQNLVAVPRSD
SVTPVTVAVITCKYPEALEQGRGDPIYLGIQNPEMCLYCEKVGEQPTLQ
LKEQKIMDLYGQPEPVKPFLFYRAKTGRTSTLESVAFPDWFIASSKRDQ
PIILTSELGKSYNTAFELNIND

In certain embodiments, the immunocytokine comprises an anti-CD3 antibody or an antigen-binding fragment thereof disclosed herein and an IL18 polypeptide. In certain embodiments, the IL18 polypeptide comprises or consists of an amino acid sequence that is at least about 80%, at least about 85%, at least about 90%, at least about 95%, at least about 96%, at least about 97%, at least about 98%, or at least about 99% identical to the amino acid sequence set forth in SEQ ID NO: 71 or a fragment thereof. In certain embodiments, the IL18 polypeptide comprises or consists of an amino acid sequence identical to the amino acid sequence set forth in SEQ ID NO: 71 or a fragment thereof.

[SEQ ID NO: 71]
MAAEPVEDNCINFVAMKFIDNTLYFIAEDDENLESDYFGKLESKLSVIR
NLNDQVLFIDQGNRPLFEDMTDSDCRDNAPRTIFIISMYKDSQPRGMAV
TISVKCEKISTLSCENKIISFKEMNPPDNIKDTKSDIIFFQRSVPGHDN
KMQFESSSYEGYFLACEKERDLFKLILKKEDELGDRSIMFTVQNED

### 4. Cells

The presently disclosed subject matter provides cells comprising a presently disclosed anti-CD3 antibody or antigen-binding fragment thereof (e.g., one disclosed in Section 3). The presently disclosed subject matter provides cells comprising a presently disclosed anti-CD3 multispecific molecule (e.g., one disclosed in Section 3.7). In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell. In certain embodiments, the pluripotent stem cell is an embryonic stem cell (ESC) or an induced pluripotent stem cell (iPSC).

In certain embodiments, the cell is a T cell. T cells can be lymphocytes that mature in the thymus and are responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR like fusion molecule. In certain embodiments, the immunoresponsive cell is a T cell. The T cell can be a CD4⁺ T cell or a CD8+ T cell. In certain embodiments, the T cell is a CD4⁺ T cell. In certain embodiments, the T cell is a CD8⁺ T cell.

In certain embodiments, the cell is a Natural Killer (NK) cell. NK cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of T cells upon encounter with targeted tumor cells.

In certain embodiments, the cells can be transduced with the presently disclosed anti-CD3 antibody, antigen-binding fragment thereof, or multispecific molecule such that the cells express the anti-CD3 antibody, antigen-binding fragment thereof, or the multispecific molecule.

In certain embodiments, the cell further comprises a second soluble antibody, a soluble antigen-binding fragment, or a fusion protein, which binds to a polypeptide having immunosuppressive activity or immunostimulatory activity. The second soluble antibody, antigen-binding fragment, or fusion protein can enhance the immune response of the cell. In certain embodiments, the second soluble antibody, antigen-binding fragment, or fusion protein is secreted from the cell. Non-limiting examples of antibodies, antigen-binding fragments, and fusion proteins include monoclonal antibodies, single-chain variable fragments (scFvs), scFv-Fc fusion proteins, bispecific antibodies, minibodies, and BiTEs. In certain embodiments, the antigen-binding fragment is a scFv. In certain embodiments, the fusion protein is a scFv-Fc fusion protein.

In certain embodiments, the second soluble antibody, antigen-binding fragment, or fusion protein binds to a polypeptide having immunosuppressive activity. In certain embodiments, the polypeptide having immunosuppressive activity is selected from the group consisting of CD47, PD-1, CTLA-4, BTLA, LAG-3, 2B4, CD47, and their corresponding ligands or receptors (including, not limited to, SIRPa, PD-L1, PD-L2, TNFRSF14, CD48, and FGL-1). In certain embodiments, the second soluble antibody, antigen-binding fragment, or fusion protein is an antagonist antibody, antigen-binding fragment, or fusion protein. In certain embodiments, the second soluble antibody, antigen-binding fragment, or fusion protein is an antagonist antibody, antigen-binding fragment, or fusion protein that binds to PD-1. In certain embodiments, the second soluble antibody, antigen-binding fragment, or fusion protein is an antagonist fusion protein that binds to PD-1. In certain embodiments, the second soluble antibody, antigen-binding fragment, or fusion protein is an antagonist scFv that binds to PD-1. In certain embodiments, the second soluble antibody, antigen-binding fragment, or fusion protein is an antagonist antibody, antigen-binding fragment, or fusion protein that binds to CTLA-4. In certain embodiments, the second soluble antibody, antigen-binding fragment, or fusion protein is an antagonist scFv-Fc fusion protein that binds to CTLA-4. In certain embodiments, the second soluble antibody, antigen-binding fragment, or fusion protein is an antagonist scFv that binds to CTLA-4.

In certain embodiments, the second soluble antibody, antigen-binding fragment, or fusion protein binds to a polypeptide having immunostimulatory activity. In certain embodiments, the polypeptide having immunostimulatory activity is selected from the group consisting of CD28, OX-40, 4-1BB, GITR, and their corresponding ligands or receptors (including, but not limited to, B7-1, B7-2, OX-40L, 4-1BBL, and GITRL). In certain embodiments, the second soluble antibody, antigen-binding fragment, or fusion protein is an agonist antibody, antigen-binding fragment, or fusion protein.

Cells comprising an antigen-recognizing receptor (e.g., a CAR) and a soluble scFv that binds a polypeptide that has immunosuppressive activity or immunostimulatory activity are disclosed in International Patent Publication No. WO 2014/134165, which is incorporated by reference herein in its entirety.

### 4.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD371, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGFIR, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1,

Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, K-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PRI), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, U5 snRNP200, and ZDHHC11.

In certain embodiments, the antigen-recognizing receptor binds to a pathogen antigen, e.g., for use in treating and/or preventing a pathogen infection. Non-limiting examples of pathogens include viruses, bacteria, fungi, parasites, and protozoans capable of causing disease.

Non-limiting examples of pathogenic viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. rcoviruses, orbiviurses and rotaviruscs); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses), human papilloma virus (i.e. HPV), JC virus, Epstein Bar Virus, Merkel cell polyoma virus.

Non-limiting examples of pathogenic bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Clostridium difficile, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 4.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains comprising a Variable (V) region and a Constant (C) region. The Constant region is proximal to the cell membrane, followed by a transmembrane region and a short cytoplasmic tail that lacks the ability to transduce a signal. The Variable region binds to the peptide/MHC complex. The variable domain of each pair (alpha/beta or gamma/delta) of TCR polypeptides comprises three complementarity determining regions (CDRs).

In certain embodiments, a TCR can form a receptor complex with three dimeric signaling modules CD3δ/ε, CD3γ/δ and CD3ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the recombinant TCR differs from any naturally occurring TCR by at least one amino acid residue. In certain embodiments, the recombinant TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the recombinant TCR is modified from a naturally occurring TCR by at least one amino acid residue. In certain embodiments, the recombinant TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

In certain embodiments, the TCR recognizes a viral antigen. In certain embodiments, the TCR is expressed in a virus-specific T cell. In certain embodiments, the virus-specific T cell is derived from an individual immune to a viral infection, e.g., BK virus, human herpesvirus 6, Epstein-Barr virus (EBV), cytomegalovirus or adenovirus. In certain embodiments, the virus-specific T cell is a T cell disclosed in Leen et al., Blood, Vol. 121, No. 26, 2013; Barker et al., Blood, Vol. 116, No. 23, 2010; Tzannou et al., Journal of Clinical Oncology, Vol. 35, No. 31, 2017; or Bollard et al., Blood, Vol. 32, No. 8, 2014, each of which is incorporated by reference in its entirety. In certain embodiments, the TCR recognizes a tumor antigen (including a TAA or TSA). In certain embodiments, the TCR is expressed in a tumor-specific T cell. In certain embodiments, the tumor-specific T cell is a tumor-infiltrating T cell generated by culturing T cells with explants of a tumor, e.g., melanoma or an epithelial cancer. In certain embodiments, the tumor-specific T cell is a T cell disclosed in Stevanovic et al, Science, 356, 200-205, 2017; Dudley et al. Journal of Immunotherapy, 26 (4): 332-342, 2003; or Goff et al, Journal of Clinical Oncology, Vol. 34, No. 20, 2016, each of which is incorporated by reference in its entirety.

### 4.3. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARS. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In accordance with the presently disclosed subject matter, a CAR comprises an extracellular antigen-binding domain, a transmembrane domain, and an intracellular signaling domain, wherein the extracellular antigen-binding domain specifically binds to an antigen, e.g., a tumor antigen (TAA or TSA) or a pathogen antigen.

### 4.3.1. Extracellular Antigen-Binding Domain of a CAR

The extracellular antigen-binding domain of the CAR binds to an antigen (e.g., one described in Section 4.1). In certain embodiments, the antigen is a tumor antigen. In certain embodiments, the antigen is a pathogen antigen. In certain embodiments, the extracellular antigen-binding domain is a single chain variable fragment (scFv). In certain embodiments, the scFv is a human scFv. In certain embodiments, the scFv is a humanized scFv. In certain embodiments, the scFv is a murine scFv. In certain embodiments, the extracellular antigen-binding domain of the CAR is a Fab, which is optionally crosslinked. In certain embodiments, the extracellular antigen-binding domain of the CAR is a F(ab)₂.

### 4.3.2. Transmembrane Domain of a CAR

In certain embodiments, the CAR comprises a transmembrane domain. In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the antigen-recognizing receptor can comprise a native or modified transmembrane domain of a CD8 polypeptide, a CD28 polypeptide, a CD3 polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain of the CAR comprises a CD28 polypeptide (e.g., the transmembrane domain of CD28 or a portion thereof). In certain embodiments, the transmembrane domain of the CAR comprises a human CD28 polypeptide (e.g., the transmembrane domain of human CD28 or a portion thereof. In certain embodiments, the CD28 polypeptide comprises or consists of an amino acid sequence that is a consecutive portion of the amino acid sequence having a NCBI Reference No: NP_006130 (SEQ ID NO: 95), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 95. In certain embodiments, the transmembrane domain of the CAR comprises a CD28 polypeptide that comprises or consists of amino acids 153 to 179 of SEQ ID NO: 95. SEQ ID NO: 95 is provided below.

[SEQ ID NO: 95]
MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS

### 4.3.3. Hinge/Spacer Region of the CAR

In certain embodiments, the CAR comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. The hinge/spacer region can be the hinge region from IgG1, or the CH2CH3 region of immunoglobulin and portions of CD3, a portion of a CD28 polypeptide (e.g., a portion of SEQ ID NO: 95), a portion of a CD8 polypeptide, a variation of any of the foregoing which is at least about 80%, at least about 85%, at least about 90%, at least about 95%, or at least about 100% homologous or identical thereto, or a synthetic spacer sequence.

In certain embodiments, the hinge/spacer region comprises a native or modified hinge region of a CD28 polypeptide. In certain embodiments, the hinge/spacer region comprises a CD28 polypeptide comprising or consisting of amino acids 114 to 152 of SEQ ID NO: 95.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8a polypeptide and the transmembrane domain comprises a CD8a polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

### 4.3.4. Intracellular Signaling Domain of a CAR

In certain embodiments, the CAR comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T-cell) after antigen is bound. The intracellular signaling domain of the CD33-chain is the primary transmitter of signals from endogenous TCRs.

In certain embodiments, the intracellular signaling domain of the CAR comprises a native CD3ζ. In certain embodiments, the native CD3ζ comprises or consists of an amino acid sequence that is at least about 85%, about 90%, about 95%, about 96%, about 97%, about 98%, about 99% or about 100% identical or homologous to the amino acid sequence having a NCBI Reference No: NP_932170 (SEQ ID NO: 96) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 98, which is at least about 20, or at least about 30, or at least about 40, or at least about 50, and up to about 164 amino acids in length. In certain embodiments, the native CD3ζ comprises or consists of the amino acid sequence of amino acids 1 to 164, 1 to 50, 50 to 100, 52 to 164, 100 to 150, or 150 to 164 of SEQ ID NO: 96. In certain embodiments, the intracellular signaling domain of the CAR comprises a native CD3 comprising or consisting of the amino acid sequence of amino acids 52 to 164 of SEQ ID NO: 96. SEQ ID NO: 96 is provided below:

[SEQ ID NO: 96]
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

In certain embodiments, the CD3ζ polypeptide comprises or consists of an amino acid sequence that is at least about 85%, about 90%, about 95%, about 96%, about 97%, about 98%, about 99% or about 100% identical or homologous to the amino acid sequence set forth in SEQ ID NO: 97 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 97, which is at least about 20, or at least about 30, or at least about 40, or at least about 50, and up to about 112 amino acids in length. In certain embodiments, the native CD33 comprises or consists of the amino acid sequence of amino acids 1 to 112, 1 to 25, 25 to 50, 50 to 100, 50 to 112, or 100 to 112 of SEQ ID NO: 97. In certain embodiments, the intracellular signaling domain of the CAR comprises a CD3ζ polypeptide comprising or consisting of the amino acid sequence set forth in SEQ ID NO: 97. SEQ ID NO: 97 is provided below:

[SEQ ID NO: 97]
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 99.

[SEQ ID NO: 99]
QNQLYNELNLGRREEYDVLDKR

An exemplary nucleotide sequence encoding the amino acid sequence of SEQ ID NO: 99 is set forth in SEQ ID NO: 100, which is provided below.

[SEQ ID NO: 100]
CAGAACCAGCTCTATAACGAGCTCAATCTAGGACGAAGAGAGGAGTACG
ATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 101, which is provided below.

[SEQ ID NO: 101]
QNQLFNELNLGRREEFDVLDKR

An exemplary nucleotide sequence encoding the amino acid sequence of SEQ ID NO: 101 is set forth in SEQ ID NO: 102, which is provided below.

[SEQ ID NO: 102]
CAGAACCAGCTCTTTAACGAGCTCAATCTAGGACGAAGAGAGGAGTTCG
ATGTTTTGGACAAGAGA

In certain embodiments, the modified CD33 polypeptide comprises a native ITAM2. In certain embodiments, the native ITAM2 comprises or consists of the amino acid sequence set forth in SEQ ID NO: 103, which is provided below.

[SEQ ID NO: 103]
QEGLYNELQKDKMAEAYSEIGMK

An exemplary nucleotide sequence encoding the amino acid sequence of SEQ ID NO: 103 is set forth in SEQ ID NO: 104, which is provided below.

[SEQ ID NO: 104]
CAGGAAGGCCTGTACAATGAACTGCAGAAAGATAAGATGGCGGAGGCCT
ACAGTGAGATTGGGATGAAA

In certain embodiments, the modified CD33 polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 105, which is provided below.

[SEQ ID NO: 105]
QEGLFNELQKDKMAEAFSEIGMK

An exemplary nucleotide sequence encoding the amino acid sequence of SEQ ID NO: 105 is set forth in SEQ ID NO: 106, which is provided below.

[SEQ ID NO: 106]
CAGGAAGGCCTGTTCAATGAACTGCAGAAAGATAAGATGGCGGAGGCCT
TCAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 107, which is provided below.

[SEQ ID NO: 107]
HDGLYQGLSTATKDTYDALHMQ

An exemplary nucleotide sequence encoding the amino acid sequence of SEQ ID NO: 107 is set forth in SEQ ID NO: 108, which is provided below.

[SEQ ID NO: 108]
CACGATGGCCTTTACCAGGGTCTCAGTACAGCCACCAAGGACACCTACG
ACGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 109, which is provided below.

[SEQ ID NO: 109]
HDGLFQGLSTATKDTFDALHMQ

An exemplary nucleotide sequence encoding the amino acid sequence of SEQ ID NO: 109 is set forth in SEQ ID NO: 110, which is provided below.

[SEQ ID NO: 110]
CACGATGGCCTTTTCCAGGGGCTCAGTACAGCCACCAAGGACACCTTCG
ACGCCCTTCACATGCAG

Various modified CD33 polypeptides and CARs comprising modified CD3° C. polypeptides are disclosed in International Patent Application Publication No. WO2019/133969, which is incorporated by reference hereby in its entirety.

In certain embodiments, the intracellular signaling domain of the CAR comprises a modified CD33 polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3 polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 99, an ITAM2 variant consisting of the amino acid sequence set forth in SEQ ID NO: 105, and an ITAM3 variant consisting of the amino acid sequence set forth in SEQ ID NO: 109. In certain embodiments, the intracellular signaling domain of the CAR is designated as “1XX”. In certain embodiments, the modified CD3ζ polypeptide comprises or consists of the amino acid sequence set forth in SEQ ID NO: 111. SEQ ID NO: 111 is provided below:

[SEQ ID NO: 111]
RVKFSRSADAPAYQQGQNQLYNELNLGRREEYDVLDKRRGRDPEMGGKP
RRKNPQEGLFNELQKDKMAEAFSEIGMKGERRRGKGHDGLFQGLSTATK
DTFDALHMQALPPR

In certain embodiments, the intracellular signaling domain of the CAR comprises a modified CD3 polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 111 or a fragment thereof, and/or may optionally comprise up to one or up to two or up to three conservative amino acid substitutions.

An exemplary nucleotide sequence encoding the amino acid sequence of SEQ ID NO: 111 is set forth in SEQ ID NO: 112, which is provided below.

[SEQ ID NO: 112]
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTTCAATGAACTGCAGAAAGATA
AGATGGCGGAGGCCTTCAGTGAGATTGGGATGAAAGGCGAGCGCCGGAG
GGGCAAGGGGCACGATGGCCTTTTCCAGGGGCTCAGTACAGCCACCAAG
GACACCTTCGACGCCCTTCACATGCAGGCCCTGCCCCCTCGC

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, TNFRSF18 (GITR, CD357), and NKG2D.

In certain embodiments, the intracellular signaling domain of the CAR comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the antigen-recognizing receptor comprise or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 95 or a fragment thereof, and/or may optionally comprise up to one or up to two or up to three conservative amino acid substitutions. In certain embodiments, the CD28 polypeptide comprised in the co-stimulatory signaling region comprises or consist of an amino acid sequence that is a consecutive portion of SEQ ID NO: 95, which is at least about 20, or at least about 30, or at least about 40, or at least about 50, and up to about 220 amino acids in length. In certain embodiments, the CD28 polypeptide comprised in the co-stimulatory signaling region comprises or consists of amino acids 1 to 220, 1 to 50, 50 to 100, 100 to 150, 114 to 220, 150 to 200, 180 to 220, or 200 to 220 of SEQ ID NO: 95. In certain embodiments, the intracellular signaling domain of the CAR comprises a co-stimulatory signaling region that comprises a CD28 polypeptide comprising or consisting of amino acids 180 to 220 of SEQ ID NO: 95.

An exemplary nucleotide sequence encoding amino acids 180 to 220 of SEQ ID NO: 95 is set forth in SEQ ID NO: 74, which is provided below.

[SEQ ID NO: 74]
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC

In certain embodiments, the intracellular signaling domain of the CAR comprises a co-stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having a NCBI Reference No.: NP_001552 (SEQ ID NO: 98) or a fragment thereof, and/or may optionally comprise up to one or up to two or up to three conservative amino acid substitutions. In certain embodiments, the 4-1BB comprised in the co-stimulatory signaling region comprises or consists of an amino acid sequence that is a consecutive portion of SEQ ID NO: 98, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the 4-1BB polypeptide comprised in the co-stimulatory signaling region comprises or consists of the amino acid sequence of amino acids 1 to 255, 1 to 50, 50 to 100, 100 to 150, 150 to 200, or 200 to 255 of SEQ ID NO: 98. In certain embodiments, the co-stimulatory signaling region comprises a 4-1BB polypeptide comprising or consisting of amino acids 214 to 255 of SEQ ID NO: 98. SEQ ID NO: 98 is provided below.

[SEQ ID NO: 98]
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
VNGTKERDVVCGPSPADLSPGASSVTPPAPAREPGHSPQIISFFLALTS
TALLFLLFFLTLRFSVVKRGRKKLLYIFKQPFMRPVQTTQEEDGCSCRF
PEEEEGGCEL

In certain embodiments, the intracellular signaling domain of the CAR comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, and NKG2D. In certain embodiments, the intracellular signaling domain of the antigen-recognizing receptor comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

### 4.4. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Bacuerle et al., “Synthetic TRUC receptors engaging the complete T cell receptor for potent anti-tumor response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}.) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

The TCR like fusion molecule can bind to a tumor antigen or a pathogen antigen. In certain embodiments, the TCR like fusion molecule binds to a tumor antigen.

### 5. Nucleic Acids Encoding the Antibodies or Antigen-Binding Fragments and/or V_{H} and/or V_{L} Thereof

The presently disclosed subject matter provides nucleic acids encoding the anti-CD3 antibodies or antigen-binding fragments thereof disclosed herein. The presently disclosed subject matter provides nucleic acids encoding the heavy chain variable region sequence of any one of the presently disclosed anti-CD3 antibodies (e.g., 01F05G01, 07B09, 12C08, 13D02, 01B08B01, 06G07A04, 08G12, 09B01, 09C02, 11A10, 11B11, 13E03, 07B10, 07B12, and 08B12 antibodies).

The presently disclosed subject matter provides nucleic acids encoding the light chain variable region sequence of any one of the presently disclosed anti-CD3 antibodies (e.g., 01F05G01, 07B09, 12C08, 13D02, 01B08B01, 06G07A04, 08G12, 09B01, 09C02, 11A10, 11B11, 13E03, 07B10, 07B12, and 08B 12 antibodies).

The presently disclosed subject matter provides nucleic acids encoding the multi-specific molecules disclosed herein (e.g., those disclosed in Section 3.7). In certain embodiments, the nucleic acid comprises or consists of a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 125, SEQ ID NO: 127, SEQ ID NO: 129, SEQ ID NO: 131, SEQ ID NO: 141, SEQ ID NO: 143, or SEQ ID NO: 145. In certain embodiments, the nucleic acid comprises or consists of a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 129, SEQ ID NO: 131, or SEQ ID NO: 145.

In certain embodiments, the nucleic acid comprises or consists of the nucleotide sequence set forth in SEQ ID NO: 125, SEQ ID NO: 127, SEQ ID NO: 129, SEQ ID NO: 131, SEQ ID NO: 141, SEQ ID NO: 143, or SEQ ID NO: 145. In certain embodiments, the nucleic acid comprises or consists of the nucleotide sequence set forth in SEQ ID NO: 129, SEQ ID NO: 131, or SEQ ID NO: 145.

Further provided are vectors comprising the presently disclosed nucleic acids. In certain embodiments, the vector is an expression vector. The presently disclosed subject matter further provides host cells comprising the vectors disclosed herein. In certain embodiments, the host cells are T cells.

### 6. Pharmaceutical Compositions and Methods of Treatment

The presently disclosed subject matter provides compositions comprising a presently disclosed anti-CD3 antibody or an antigen-binding fragment thereof, a presently disclosed immunoconjugate, a presently disclosed multi-specific molecule, or a presently disclosed immunocytokine. The presently disclosed subject matter also provides compositions comprising a presently disclosed cell or cells (e.g., those disclosed in Section 4). In certain embodiments, the composition is a pharmaceutical composition further comprising a pharmaceutically acceptable carrier.

Suitable pharmaceutically acceptable carriers include, for example, one or more of water, saline, phosphate buffered saline, dextrose, glycerol, ethanol, and the like, as well as combinations thereof. Pharmaceutically acceptable carriers may further comprise minor amounts of auxiliary substances such as wetting or emulsifying agents, preservatives, or buffers, which enhance the shelf life or effectiveness of the binding proteins. The compositions of the injection can, as is well known in the art, be formulated so as to provide quick, sustained, or delayed release of the active ingredient after administration to the mammal.

The presently disclosed subject matter provides various methods of using the anti-CD3 antibodies or antigen-binding fragments thereof, the immunoconjugate, the multi-specific molecule, the cell, and the composition disclosed herein. For example, the presently disclosed subject matter provides methods for treating or ameliorating a disease or disorder in a subject. In certain embodiments, the method comprises administering one or more of the anti-CD3 antibodies or antigen-binding fragments thereof, the immunoconjugate, the multi-specific molecule, the cell, or the composition disclosed herein to the subject. In certain embodiments, the disease or disorder is a tumor.

The presently disclosed subject matter provides methods of reducing tumor burden in a subject. In certain embodiments, the method comprises administering one or more of the anti-CD3 antibodies or antigen-binding fragments thereof, the immunoconjugate, the multi-specific molecule, the cell, or the composition disclosed herein to the subject. The presently disclosed anti-CD3 antibodies or antigen-binding fragments thereof can reduce the number of tumor cells, reduce tumor size, and/or eradicate the tumor in the subject.

The presently disclosed subject matter also provides methods of increasing or lengthening survival of a subject having a tumor. In certain embodiments, the method comprises administering one or more of the anti-CD3 antibodies or antigen-binding fragments thereof, the immunoconjugate, the multi-specific molecule, the cell, or the composition disclosed herein to the subject. The method can reduce or eradicate tumor burden in the subject.

The presently disclosed subject matter further provides methods for treating and/or preventing a tumor in a subject. In certain embodiments, the method comprises administering one or more of the anti-CD3 antibodies or antigen-binding fragments thereof, the immunoconjugate, the multi-specific molecule, the cell, or the composition disclosed herein to the subject.

Such methods comprise administering the presently disclosed anti-CD3 antibodies or antigen-binding fragments thereof in an amount effective, a presently disclosed composition (e.g., a pharmaceutical composition) to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the tumor is a solid tumor. In solid tumor immunotherapy, lack of a targetable antigen that is uniformly overexpressed on cancer cells can be overcome by antigen-independent cytotoxic strategics.

In certain embodiments, the tumor is a hematological tumor. In certain embodiments, the hematological tumor is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms. In certain embodiments, the hematological tumor is a myelodysplastic syndromes (MDS).

Non-limiting examples of hematological tumors include leukemias and lymphomas (e.g., Hodgkin lymphoma, non-Hodgkin's lymphoma, and B-cell lymphomas). In certain embodiments, the tumor is cancer. In certain embodiments, the tumor is a hematological malignancy. Non-limiting examples of tumors include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm (e.g., malignant neoplasm) is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer (including non-small cell lung cancer (NSCLC) and small cell lung cancer (SCLC), pancreatic cancer, prostate cancer, skin cancer, stomach cancer, glioblastoma, and throat cancer.

Non-limiting examples of solid tumors glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, non-small cell lung cancer (NSCLC), lung adenocarcinoma, stomach adenocarcinoma, cervical and endocervical cancer, kidney clear cell carcinoma, and testicular germ cell tumors. In certain embodiments, the tumor is acute myeloid leukemia (AML).

The presently disclosed subject matter provides methods for treating and/or preventing an autoimmune disease in a subject. The method can comprise administering one or more of the anti-CD3 antibodies or antigen-binding fragments thereof, the immunoconjugate, the multi-specific molecule, or the composition disclosed herein to a subject having an autoimmune disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myclodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, ostcomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Any suitable method or route can be used to administer a presently disclosed anti-CD3 antibody, and optionally, to co-administer antineoplastic agents. Routes of administration include, but are not limited to, oral, intravenous, intraperitoneal, subcutaneous, intramuscular, intranodal, intratumoral, intraosscous, intrathecal, pleural, intrapleural, topical, and direct administration. It should be emphasized, however, that the presently disclosed subject matter is not limited to any particular method or route of administration.

The presently disclosed anti-CD3 antibodies or antigen-binding fragments thereof can be administered as a conjugate, which binds specifically to the receptor and delivers a toxic, lethal payload following ligand-toxin internalization.

In certain embodiments, the presently disclosed methods include administering the anti-CD3 antibodies or antigen-binding fragments thereof, the immunoconjugate, the multi-specific molecule, the cell, or the composition disclosed herein in combination with a second therapeutic agent. As used here, the term “in combination with” means that the anti-CD3 antibodies or antigen-binding fragments thereof, the immunoconjugate, the multi-specific molecule, or the composition disclosed herein, and one or more agents, e.g., a second therapeutic agent, are administered to a subject as part of a treatment regimen or plan.

In certain embodiments, the second therapeutic agent targets components of the immune system to fight cancer can be used with the presently disclosed methods. Non-limiting examples of second therapeutic agents include immune checkpoint inhibitors, dendritic cells, therapeutic antibodies (e.g., anti-CD33 antibodies, anti-CD11b antibodies), cancer vaccines, cytokines (e.g., IL-12, GM-CSF, IL-2, IFNβ, IFNγ, MIP-1, MCP-1, IL-8), Bacillus Calmette-Guérin (BCG), and any combinations thereof. In certain embodiments, the second therapeutic agent is an immune checkpoint inhibitor. In certain embodiments, the immune checkpoint inhibitor is selected from anti-PD1 antibodies, anti-PD-L1 antibodies, anti-CTLA-4 antibodies, anti-BTLA antibodies, anti-TIM3 antibodies, anti-LAG-3 antibodies, and any combinations thereof. For example, but without any limitation, the immune checkpoint inhibitor can be pembrolizumab (KEYTRUDA®), nivolumab (OPDIVO®), comiplimab (LIBTAYO®), atezolizumab (TECENTRIQ®), avelumab (BAVENCIO®), durvalumab (IMFINZI®), or ipilimumab (YERVOY®).

In certain embodiments, the second therapeutic agent is a chemotherapy. For example, but without any limitation, chemotherapy includes CHOP (cyclophosphamide, doxorubicin, vincristie, prednisonc), EPOCH (etoposide, vincristine, doxorubicin, cyclophosphamide, prednisone), or any other multidrug regimens.

In certain embodiments, the anti-CD3 antibodies or antigen-binding fragments thereof, the immunoconjugate, the multi-specific molecule, the cell, or the composition disclosed herein and the second therapeutic agent can be administered to the subject as part of a treatment regimen. In certain embodiments, the anti-CD3 antibodies or antigen-binding fragments thereof, the immunoconjugate, the multi-specific molecule, the cell, or the composition disclosed herein and the second therapeutic agent can be administered concurrently to the subject. In certain embodiments, the anti-CD3 antibodies or antigen-binding fragments thereof, the immunoconjugate, the multi-specific molecule, the cell, or the composition disclosed herein and the second therapeutic agent can be administered at the same time. In certain embodiments, the anti-CD3 antibodies or antigen-binding fragments thereof, the immunoconjugate, the multi-specific molecule, the cell, or the composition disclosed herein and the second therapeutic agent can be administered sequentially in any order (e.g., the anti-CD3 antibodies or antigen-binding fragments thereof, the immunoconjugate, the multi-specific molecule, or the composition disclosed herein are administered to the subject after the second therapeutic agent is administered) or at different points in time (e.g., the anti-CD3 antibodies or antigen-binding fragments thereof, the immunoconjugate, the multi-specific molecule, the cell, or the composition disclosed herein and the second therapeutic agent are administered to the subject on the same day but different hours; or the anti-CD3 antibodies or antigen-binding fragments thereof, the immunoconjugate, the multi-specific molecule, the cell, or the composition disclosed herein and the second therapeutic agent are administered to the subject in the same week but on different days).

The presently disclosed subject matter further provides methods for myeloid depleting a subject before receiving a treatment (e.g., an adoptive cell therapy). In certain embodiments, the method comprises administering one or more of the anti-CD3 antibodies or antigen-binding fragments thereof, the immunoconjugate, the multi-specific molecule, the cell, or the composition disclosed herein to the subject. In certain embodiments, the method comprises administering the multi-specific molecule or the composition disclosed herein to the subject. In certain embodiments, the multi-specific molecule includes a second binding specificity for CD33, U5 snRNP200, or CD371.

### 7. Diagnostic and Prognostic Methods

The presently disclosed anti-CD3 antibodies, antigen-binding fragments thereof, multi-specific molecules, immunocytokines, and nucleic acids encoding the same can be used for diagnostic and prognostic applications as well as use as research tools for detection of CD3ε in a biological sample, in a cell, a tissue, or a blood sample. The presently disclosed subject matter provides methods for detecting CD3ε in a cell, a tissue, or a blood sample. In certain embodiments, the method comprises: contacting a cell, a tissue, or a blood sample with the antibody, antigen-binding fragment thereof, or multi-specific molecule disclosed herein, wherein the antibody, antigen-binding fragment thereof or multi-specific molecule comprises a detectable label; and determining the amount of the labeled antibody, antigen-binding fragment thereof, or multi-specific molecule bound to the cell, tissue, or blood sample by measuring the amount of detectable label associated with the cell or tissue, wherein the amount of bound antibody, antigen-binding fragment thereof, or multi-specific molecule indicates the amount of CD3ε in the cell, tissue, or a blood sample. The cell or tissue can be any cell or tissue, including any normal, healthy, or cancerous cells and tissues. In certain embodiments, the blood sample is a peripheral blood sample.

The presently disclosed anti-CD3 antibodies or antigen-binding fragments thereof can be used in methods known in the art relating to the localization and/or quantitation of CD3ε polypeptides (e.g., for use in measuring levels of the CD3E protein within appropriate physiological samples, for use in diagnostic methods, for use in imaging the polypeptide, and the like). The presently disclosed anti-CD3 antibodies or antigen-binding fragments thereof can be used to isolate a CD3ε polypeptide by standard techniques, such as affinity chromatography or immunoprecipitation. The presently disclosed anti-CD3 antibodies or antigen-binding fragments thereof can facilitate the purification of natural immunoreactive

CD3ε proteins from biological samples, e.g., mammalian sera or cells as well as recombinantly-produced immunoreactive CD3ε proteins expressed in a host system. Moreover, anti-CD3 antibodies of the present technology can be used to detect an immunoreactive CD3& protein (e.g., in plasma, a cellular lysate, or cell supernatant) in order to evaluate the abundance and pattern of expression of the immunoreactive polypeptide. The presently disclosed anti-CD3 antibodies or antigen-binding fragments thereof can be used diagnostically to monitor immunoreactive CD3ε protein levels in tissue as part of a clinical testing procedure, e.g., to determine the efficacy of a given treatment regimen. As noted above, the detection can be facilitated by coupling (i.e., physically linking) the presently disclosed anti-CD3 antibodies or antigen-binding fragments thereof to a detectable substance.

An exemplary method for detecting the presence or absence of an immunoreactive CD3ε protein in a biological sample comprises contacting a biological sample from a subject with a presently disclosed anti-CD3 antibody or an antigen-binding fragment thereof, wherein the presence of an immunoreactive CD3ε protein is detected in the biological sample. Detection may be accomplished by means of a detectable label attached to the antibody.

The term “labeled” with regard to the anti-CD3 antibody or antigen-binding fragment thereof is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In certain embodiments, the presently disclosed anti-CD3 antibodies or antigen-binding fragments thereof are conjugated to one or more detectable labels. For such uses, the presently disclosed anti-CD3 antibodies or antigen-binding fragments thereof may be detectably labeled by covalent or non-covalent attachment of a chromogenic, enzymatic, radioisotopic, isotopic, fluorescent, toxic, chemiluminescent, nuclear magnetic resonance contrast agent or other label.

The presently disclosed detection methods can be used to detect an immunoreactive CD3ε protein in a biological sample in vitro as well as in vivo. Non-limiting examples of in vitro techniques for detection of an immunoreactive CD3ε protein include enzyme linked immunosorbent assays (ELISAs), Western blots, immunoprecipitations, radioimmunoassay, and immunofluorescence. Furthermore, in vivo techniques for detection of an immunoreactive CD3& protein include introducing into a subject a labeled anti-CD3 antibody or an antigen-binding fragment thereof. For example, the anti-CD3 antibody or antigen-binding fragment thereof can be labeled with a radioactive marker whose presence and location in a subject can be detected by standard imaging techniques. In certain embodiments, the biological sample comprises CD3& protein molecules from the test subject.

The presently disclosed anti-CD3 antibodies or antigen-binding fragments thereof can be used to assay immunoreactive CD3& protein levels in a biological sample (e.g., human plasma) using antibody-based techniques. For example, protein expression in tissues can be studied with classical immunohistological methods. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (³H), indium (¹¹¹In), and technetium (^{99m}Tc), and fluorescent labels, such as fluorescein, rhodamine, and green fluorescent protein (GFP), as well as biotin.

In addition to assaying immunoreactive CD3ε protein levels in a biological sample, the presently disclosed anti-CD3 antibodies or antigen-binding fragments thereof may be used for in vivo imaging of CD38. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the anti-CD3 antibodies by labeling of nutrients for the relevant scFv clone.

The presently disclosed anti-CD3 antibodies or antigen-binding fragments thereof, which are labeled with an appropriate detectable imaging moiety (such as a radioisotope (e.g., ¹³¹I, ¹¹¹IN ^{99m}Tc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled anti-CD3 antibody or antigen-binding fragment thereof then accumulates at the location of cells which contains the specific target polypeptide. For example, the labeled anti-CD3 antibodies or antigen-binding fragments thereof accumulate within the subject in cells and tissues in which the CD3ε protein has localized.

Thus, the presently disclosed subject matter provides diagnostic methods of a medical condition. In certain embodiments, the method comprises: (a) assaying the expression of immunoreactive CD3ε protein by measuring the binding of a presently disclosed anti-CD3 antibody or an antigen-binding fragment thereof in cells or body fluid of an individual; and (b) comparing the amount of immunoreactive CD3ε protein present in the sample with a standard reference, wherein an increase or decrease in immunoreactive CD3ε protein levels compared to the standard is indicative of a medical condition.

Furthermore, the presently disclosed anti-CD3 antibodies or antigen-binding fragments thereof may be used to purify immunoreactive CD3& protein from a sample. In certain embodiments, the antibodies are immobilized on a solid support. Non-limiting examples of such solid supports include plastics such as polycarbonate, complex carbohydrates such as agarose and sepharose, acrylic resins and such as polyacrylamide and latex beads. Techniques for coupling antibodies to such solid supports are well known in the art.

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimalcimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl) propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

An antibody or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-crythrole and tris-hydroxy-aminomethane.

Noncovalent Binding Association. An antibody or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs known to those skilled in the art.

The presently disclosed anti-CD3 antibodies or antigen-binding fragments thereof are useful in diagnostic methods. As such, the presently disclosed subject matter provides methods using the presently disclosed anti-CD3 antibodies or antigen-binding fragments thereof in diagnosis of CD3ε activity in a subject. The presently disclosed anti-CD3 antibodies or antigen-binding fragments thereof may be selected such that they have any level of epitope binding specificity and high binding affinity to a CD3& polypeptide.

The presently disclosed anti-CD3 antibodies or antigen-binding fragments thereof can be used to detect an immunoreactive CD3ε protein in a variety of standard assay formats. Such formats include immunoprecipitation, Western blotting, ELISA, radioimmunoassay, and immunometric assays. Biological samples can be obtained from any tissue or body fluid of a subject. In certain embodiments, the subject is at an early stage of cancer. In certain embodiments, the early stage of cancer is determined by the level or expression pattern of CD3ε protein in a sample obtained from the subject. In certain embodiments, the sample is selected from the group consisting of urine, blood, scrum, plasma, saliva, amniotic fluid, cerebrospinal fluid (CSF), and biopsied body tissue.

Immunometric or sandwich assays are one format for the diagnostic methods of the present technology. Such assays use one antibody, e.g., the anti-CD3 antibody or a population of anti-CD3 antibodies immobilized to a solid phase, and another anti-CD3 antibody or a population of anti-CD3 antibodies in solution. Typically, the solution anti-CD3 antibody or population of anti-CD3 antibodies is labeled. If an antibody population is used, the population can contain antibodies binding to different epitope specificities within the target polypeptide. Accordingly, the same population can be used for both solid phase and solution antibody. If anti-CD3 monoclonal antibodies are used, first and second CD3ε monoclonal antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the CD3& protein with the anti-CD3 antibody, a sample is incubated for a period that usually varies from about 10 min to about 24 hr and is usually about 1 hr. A wash step is then performed to remove components of the sample not specifically bound to the anti-CD3 antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody. Usually for a given pair of antibodies or populations of antibodies and given reaction conditions, a calibration curve is prepared from samples containing known concentrations of target antigen. Concentrations of the immunoreactive CD3ε protein in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the CD3& protein in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, anti-CD3 antibodies can be joined to a linker molecule, such as biotin for attachment to a surface bound linker, such as avidin.

In certain embodiments, the presently disclosed anti-CD3 antibody or antigen-binding fragment thereof is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed anti-CD3 antibodies or antigen-binding fragments thereof using standard chemistries. The chelate is normally linked to the antibody by a group which enables formation of a bond to the molecule with minimal loss of immunoreactivity and minimal aggregation and/or internal cross-linking.

### 8. Kits

The presently disclosed subject matter provides kits for treatment or ameliorating a disease or disorder associated with CD3ε (e.g., a T cell), and/or detecting CD38. In certain embodiments, the kit comprises the anti-CD3 antibodies or antigen-binding fragments thereof, the immunoconjugate, the multi-specific molecule, the cell, or the composition disclosed herein. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the anti-CD3 antibodies or antigen-binding fragments thereof, the immunoconjugate, the multi-specific molecule, the cell, or the composition disclosed herein to a subject in need the treatment. The instructions can generally include information about the use of the anti-CD3 antibodies or antigen-binding fragments thereof, the immunoconjugate, the multi-specific molecule, the cell, and the composition disclosed herein for the treatment or ameliorating a disease or disorder. In certain embodiments, the instructions include at least one of the following: description of the therapeutic agent; dosage schedule and administration for treatment and/or prevention of a tumor or neoplasm or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 9. Exemplary Embodiments

Clause 1. An anti-CD3 antibody or an antigen-binding fragment thereof, comprising: (a) a heavy chain variable region comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, a CDR3 comprising the amino acid sequence set forth in SEQ ID NO: 3 or a conservative modification thereof; and a light chain variable region comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof; (b) a heavy chain variable region comprising CDR1 comprising the amino acid sequence set forth in SEQ ID NO: 9 or a conservative modification thereof, a CDR2 comprising an amino acid sequence set forth in SEQ ID NO: 10 or a conservative modification thereof, a CDR3 comprising the amino acid sequence set forth in SEQ ID NO: 11 or a conservative modification thereof; and a light chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 14 or a conservative modification thereof; (c) a heavy chain variable region comprising CDR1 comprising the amino acid sequence set forth in SEQ ID NO: 17 or a conservative modification thereof, a CDR2 comprising an amino acid sequence set forth in SEQ ID NO: 18 or a conservative modification thereof, a CDR3 comprising the amino acid sequence set forth in SEQ ID NO: 19 or a conservative modification thereof; and a light chain variable region comprising a CDR1 comprising the amino acid sequence set forth in SEQ ID NO: 20 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof; (d) a heavy chain variable region comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, a CDR3 comprising the amino acid sequence set forth in SEQ ID NO: 26 or a conservative modification thereof; and a light chain variable region comprising a CDR1 comprising the amino acid sequence set forth in SEQ ID NO: 27 or a conservative modification thereof, a CDR2 comprising the amino acid sequence set forth in SEQ ID NO: 28 or a conservative modification thereof, and a CDR3 comprising the amino acid sequence set forth in SEQ ID NO: 29 or a conservative modification thereof; (e) a heavy chain variable region comprising CDR1 comprising the amino acid sequence set forth in SEQ ID NO: 32 or a conservative modification thereof, a CDR2 comprising an amino acid sequence set forth in SEQ ID NO: 33 or a conservative modification thereof, a CDR3 comprising the amino acid sequence set forth in SEQ ID NO: 34 or a conservative modification thereof; and a light chain variable region comprising a CDR1 comprising the amino acid sequence set forth in SEQ ID NO: 35 or a conservative modification thereof, a CDR2 comprising the amino acid sequence set forth in SEQ ID NO: 36 or a conservative modification thereof, and a CDR3 comprising the amino acid sequence set forth in SEQ ID NO: 37 or a conservative modification thereof; (f) a heavy chain variable region comprising a CDR1 comprising the amino acid sequence set forth in SEQ ID NO: 40 or a conservative modification thereof, a CDR2 comprising the amino acid sequence set forth in SEQ ID NO: 18 or a conservative modification thereof, a CDR3 comprising the amino acid sequence set forth in SEQ ID NO: 41 or a conservative modification thereof; and a light chain variable region comprising a CDR1 comprising the amino acid sequence set forth in SEQ ID NO: 42 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof; (g) a heavy chain variable region comprising CDR1 comprising the amino acid sequence set forth in SEQ ID NO: 45 or a conservative modification thereof, a CDR2 comprising an amino acid sequence set forth in SEQ ID NO: 18 or a conservative modification thereof, a CDR3 comprising the amino acid sequence set forth in SEQ ID NO: 46 or a conservative modification thereof; and a light chain variable region comprising a CDR1 comprising the amino acid sequence set forth in SEQ ID NO: 20 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof; (h) a heavy chain variable region comprising CDR1 comprising the amino acid sequence set forth in SEQ ID NO: 24 or a conservative modification thereof, a CDR2 comprising an amino acid sequence set forth in SEQ ID NO: 49 or a conservative modification thereof, a CDR3 comprising the amino acid sequence set forth in SEQ ID NO: 26 or a conservative modification thereof; and a light chain variable region comprising a CDR1 comprising the amino acid sequence set forth in SEQ ID NO: 20 or a conservative modification thereof, a CDR2 comprising the amino acid sequence set forth in SEQ ID NO: 28 or a conservative modification thereof, and a CDR3 comprising the amino acid sequence set forth in SEQ ID NO: 50 or a conservative modification thereof; (i) a heavy chain variable region comprising a CDR1 comprising the amino acid sequence set forth in SEQ ID NO: 9 or a conservative modification thereof, a CDR2 comprising the amino acid sequence set forth in SEQ ID NO: 18 or a conservative modification thereof, a CDR3 comprising the amino acid sequence set forth in SEQ ID NO: 46 or a conservative modification thereof; and a light chain variable region comprising a CDR1 comprising the amino acid sequence set forth in SEQ ID NO: 53 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof; (j) a heavy chain variable region comprising CDR1 comprising the amino acid sequence set forth in SEQ ID NO: 9 or a conservative modification thereof, a CDR2 comprising an amino acid sequence set forth in SEQ ID NO: 18 or a conservative modification thereof, a CDR3 comprising the amino acid sequence set forth in SEQ ID NO: 46 or a conservative modification thereof; and a light chain variable region comprising a CDR1 comprising the amino acid sequence set forth in SEQ ID NO: 12 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof; (k) a heavy chain variable region comprising CDR1 comprising the amino acid sequence set forth in SEQ ID NO: 9 or a conservative modification thereof, a CDR2 comprising an amino acid sequence set forth in SEQ ID NO: 18 or a conservative modification thereof, a CDR3 comprising the amino acid sequence set forth in SEQ ID NO: 4 or a conservative modification thereof 6; and a light chain variable region comprising a CDR1 comprising the amino acid sequence set forth in SEQ ID NO: 58 or a conservative modification thereof, a CDR2 comprising the amino acid sequence set forth in SEQ ID NO: 13 or a conservative modification thereof, and a CDR3 comprising the amino acid sequence set forth in SEQ ID NO: 21 or a conservative modification thereof; or (1) a heavy chain variable region comprising a CDR1 comprising the amino acid sequence set forth in SEQ ID NO: 61 or a conservative modification thereof, a CDR2 comprising the amino acid sequence set forth in SEQ ID NO: 62 or a conservative modification thereof, a CDR3 comprising the amino acid sequence set forth in SEQ ID NO: 63 or a conservative modification thereof; and a light chain variable region comprising a CDR1 comprising the amino acid sequence set forth in SEQ ID NO: 4 or a conservative modification thereof, a CDR2 comprising the amino acid sequence set forth in SEQ ID NO: 64 or a conservative modification thereof, and a CDR3 comprising the amino acid sequence set forth in SEQ ID NO: 65 or a conservative modification thereof.

Clause 2. The anti-CD3 antibody or antigen-binding fragment thereof of clause 1, comprising: (a) a heavy chain variable region comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a heavy chain variable region comprising CDR1 comprising the amino acid sequence set forth in SEQ ID NO: 9, a CDR2 comprising an amino acid sequence set forth in SEQ ID NO: 10, a CDR3 comprising the amino acid sequence set forth in SEQ ID NO: 11; and a light chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; (c) a heavy chain variable region comprising CDR1 comprising the amino acid sequence set forth in SEQ ID NO: 17, a CDR2 comprising an amino acid sequence set forth in SEQ ID NO: 18, a CDR3 comprising the amino acid sequence set forth in SEQ ID NO: 19; and a light chain variable region comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21; (d) a heavy chain variable region comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a light chain variable region comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29; (e) a heavy chain variable region comprising CDR1 comprising the amino acid sequence set forth in SEQ ID NO: 32, a CDR2 comprising an amino acid sequence set forth in SEQ ID NO: 33, a CDR3 comprising the amino acid sequence set forth in SEQ ID NO: 34; and a light chain variable region comprising a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 37; (f) a heavy chain variable region comprising a CDR1 comprising the amino acid sequence set forth in SEQ ID NO: 40, a CDR2 comprising the amino acid sequence set forth in SEQ ID NO: 18, a CDR3 comprising the amino acid sequence set forth in SEQ ID NO: 41; and a light chain variable region comprising a CDR1 comprising the amino acid sequence set forth in SEQ ID NO: 42, a CDR2 comprising the amino acid sequence set forth in SEQ ID NO: 13, and a CDR3 comprising the amino acid sequence set forth in SEQ ID NO: 21; (g) a heavy chain variable region comprising CDR1 comprising the amino acid sequence set forth in SEQ ID NO: 45, a CDR2 comprising an amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21; (h) a heavy chain variable region comprising CDR1 comprising the amino acid sequence set forth in SEQ ID NO: 24, a CDR2 comprising an amino acid sequence set forth in SEQ ID NO: 49, a CDR3 comprising the amino acid sequence set forth in SEQ ID NO: 26; and a light chain variable region comprising a CDR1 comprising the amino acid sequence set forth in SEQ ID NO: 20, a CDR2 comprising the amino acid sequence set forth in SEQ ID NO: 28, and a CDR3 comprising the amino acid sequence set forth in SEQ ID NO: 50; (i) a heavy chain variable region comprising a CDR1 comprising the amino acid sequence set forth in SEQ ID NO: 9, a CDR2 comprising the amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 53, a CDR2 comprising the amino acid sequence set forth in SEQ ID NO: 13, and a CDR3 comprising the amino acid sequence set forth in SEQ ID NO: 21; (j) a heavy chain variable region comprising CDR1 comprising the amino acid sequence set forth in SEQ ID NO: 9, a CDR2 comprising an amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 21; (k) a heavy chain variable region comprising CDR1 comprising the amino acid sequence set forth in SEQ ID NO: 9, a CDR2 comprising an amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 58, a CDR2 comprising the amino acid sequence set forth in SEQ ID NO: 13, and a CDR3 comprising the amino acid sequence set forth in SEQ ID NO: 21; or (1) a heavy chain variable region comprising a CDR1 comprising the amino acid sequence set forth in SEQ ID NO: 61, a CDR2 comprising the amino acid sequence set forth in SEQ ID NO: 62, a CDR3 comprising the amino acid sequence set forth in SEQ ID NO: 63; and a light chain variable region comprising a CDR1 comprising the amino acid sequence set forth in SEQ ID NO: 4, a CDR2 comprising the amino acid sequence set forth in SEQ ID NO: 64, and a CDR3 comprising the amino acid sequence set forth in SEQ ID NO: 65.

Clause 3. The anti-CD3 antibody or antigen-binding fragment thereof of clause 2, comprising: (a) a heavy chain variable region comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a heavy chain variable region comprising CDR1 comprising the amino acid sequence set forth in SEQ ID NO: 9, a CDR2 comprising an amino acid sequence set forth in SEQ ID NO: 10, a CDR3 comprising the amino acid sequence set forth in SEQ ID NO: 11; and a light chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; (c) a heavy chain variable region comprising CDR1 comprising the amino acid sequence set forth in SEQ ID NO: 17, a CDR2 comprising an amino acid sequence set forth in SEQ ID NO: 18, a CDR3 comprising the amino acid sequence set forth in SEQ ID NO: 19; and a light chain variable region comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21; or (d) a heavy chain variable region comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a light chain variable region comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29.

Clause 4. The anti-CD3 antibody or antigen-binding fragment thereof of any one of clauses 1-3, comprising a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 15, SEQ ID NO: 22, SEQ ID NO: 30, SEQ ID NO: 38, SEQ ID NO: 43, SEQ ID NO: 47, SEQ ID NO: 51, SEQ ID NO: 54, SEQ ID NO: 56, SEQ ID NO: 59, SEQ ID NO: 66, or SEQ ID NO: 70.

Clause 5. The anti-CD3 antibody or antigen-binding fragment thereof of any one of clauses 1-3, comprising a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8, SEQ ID NO: 16, SEQ ID NO: 23, SEQ ID NO: 31, SEQ ID NO: 39, SEQ ID NO: 44, SEQ ID NO: 48, SEQ ID NO: 52, SEQ ID NO: 55, SEQ ID NO: 57, SEQ ID NO: 60, or SEQ ID NO: 67.

Clause 6. The anti-CD3 antibody or antigen-binding fragment thereof of any one of clauses 1-3, comprising: (a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, SEQ ID NO: 15, SEQ ID NO: 22, SEQ ID NO: 30, SEQ ID NO: 38, SEQ ID NO: 43, SEQ ID NO: 47, SEQ ID NO: 51, SEQ ID NO: 54, SEQ ID NO: 56, SEQ ID NO: 59, SEQ ID NO: 66, or SEQ ID NO: 70; or (b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 8, SEQ ID NO: 16, SEQ ID NO: 23, SEQ ID NO: 31, SEQ ID NO: 39, SEQ ID NO: 44, SEQ ID NO: 48, SEQ ID NO: 52, SEQ ID NO: 55, SEQ ID NO: 57, SEQ ID NO: 60, or SEQ ID NO: 67.

Clause 7. The anti-CD3 antibody or antigen-binding fragment thereof of any one of clauses 1-6, comprising a heavy chain variable region and a light chain variable region, wherein the heavy chain variable region and the light chain variable region are selected from the group consisting of: (a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8; (b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 15, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 16; (c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 22, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 23; (d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 30, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 31; (e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 38, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 39; (f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 43, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 44; (g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 47, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 48; (h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 51, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 52; (i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 54, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 55; (j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 56, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 57; (k) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 59, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 60; and (1) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 66, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 67.

Clause 8. The anti-CD3 antibody or antigen-binding fragment thereof of any one of clauses 1-7, comprising a heavy chain variable region comprising the amino acid sequence set forth in SEQ ID NO: 7, SEQ ID NO: 15, SEQ ID NO: 22, SEQ ID NO: 30, SEQ ID NO: 38, SEQ ID NO: 43, SEQ ID NO: 47, SEQ ID NO: 51, SEQ ID NO: 54, SEQ ID NO: 56, SEQ ID NO: 59, SEQ ID NO: 66, or SEQ ID NO: 70.

Clause 9. The anti-CD3 antibody or antigen-binding fragment thereof of any one of clauses 1-7, comprising a light chain variable region comprising the amino acid sequence set forth in SEQ ID NO: 8, SEQ ID NO: 16, SEQ ID NO: 23, SEQ ID NO: 31, SEQ ID NO: 39, SEQ ID NO: 44, SEQ ID NO: 48, SEQ ID NO: 52, SEQ ID NO: 55, SEQ ID NO: 57, SEQ ID NO: 60, or SEQ ID NO: 67.

Clause 10. The anti-CD3 antibody or antigen-binding fragment thereof of any one of clauses 1-9, comprising: (a) a heavy chain variable region comprising the amino acid sequence set forth in SEQ ID NO: 7, SEQ ID NO: 15, SEQ ID NO: 22, SEQ ID NO: 30, SEQ ID NO: 38, SEQ ID NO: 43, SEQ ID NO: 47, SEQ ID NO: 51, SEQ ID NO: 54, SEQ ID NO: 56, SEQ ID NO: 59, or SEQ ID NO: 66; and (b) a light chain variable region comprising the amino acid sequence set forth in SEQ ID NO: 8, SEQ ID NO: 16, SEQ ID NO: 23, SEQ ID NO: 31, SEQ ID NO: 39, SEQ ID NO: 44, SEQ ID NO: 48, SEQ ID NO: 52, SEQ ID NO: 55, SEQ ID NO: 57, SEQ ID NO: 60, or SEQ ID NO: 67.

Clause 11. The anti-CD3 antibody or antigen-binding fragment thereof of any one of clauses 1-10, wherein: (a) the heavy chain variable region comprises the amino acid sequence set forth in SEQ ID NO: 7, and the light chain variable region comprises the amino acid sequence set forth in SEQ ID NO: 8; (b) the heavy chain variable region comprises the amino acid sequence set forth in SEQ ID NO: 15, and the light chain variable region comprises the amino acid sequence set forth in SEQ ID NO: 16; (c) the heavy chain variable region comprises the amino acid sequence set forth in SEQ ID NO: 22, and the light chain variable region comprises the amino acid sequence set forth in SEQ ID NO: 23; (d) the heavy chain variable region comprises the amino acid sequence set forth in SEQ ID NO: 30, and the light chain variable region comprises the amino acid sequence set forth in SEQ ID NO: 31; (c) the heavy chain variable region comprises the amino acid sequence set forth in SEQ ID NO: 38, and the light chain variable region comprises the amino acid sequence set forth in SEQ ID NO: 39; (f) the heavy chain variable region comprises the amino acid sequence set forth in SEQ ID NO: 43, and the light chain variable region comprises the amino acid sequence set forth in SEQ ID NO: 44; (g) the heavy chain variable region comprises the amino acid sequence set forth in SEQ ID NO: 47, and the light chain variable region comprises the amino acid sequence set forth in SEQ ID NO: 48; (h) the heavy chain variable region comprises the amino acid sequence set forth in SEQ ID NO: 51, and the light chain variable region comprises the amino acid sequence set forth in SEQ ID NO: 52; (i) the heavy chain variable region comprises the amino acid sequence set forth in SEQ ID NO: 54, and the light chain variable region comprises the amino acid sequence set forth in SEQ ID NO: 55; (j) the heavy chain variable region comprises the amino acid sequence set forth in SEQ ID NO: 56, and the light chain variable region comprises the amino acid sequence set forth in SEQ ID NO: 57; (k) the heavy chain variable region comprises the amino acid sequence set forth in SEQ ID NO: 59, and the light chain variable region comprises the amino acid sequence set forth in SEQ ID NO: 60; or (1) the heavy chain variable region comprises the amino acid sequence set forth in SEQ ID NO: 66, and the light chain variable region comprises the amino acid sequence set forth in SEQ ID NO: 67.

Clause 12. The antibody or antigen-binding fragment thereof of any one of clauses 1-11, wherein one or more of the CDR sequences have up to about 5 amino acid substitutions.

Clause 13. The antibody or antigen-binding fragment thereof of any one of clauses 1-12, wherein one or more of the CDR sequences have up to about 3 amino acid substitutions.

Clause 14. The anti-CD3 antibody or antigen-binding fragment thereof of any one of clauses 1-13, wherein the antibody comprises a comprises a heavy chain constant region and/or a light chain constant region.

Clause 15. The anti-CD3 antibody or antigen-binding fragment thereof of clause 14, wherein: (a) the heavy chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 82, SEQ ID NO: 83, SEQ ID NO: 84, or SEQ ID NO: 85; and/or (b) the light chain constant region comprises an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 86.

Clause 16. The anti-CD3 antibody or antigen-binding fragment thereof of clause 14 or 15, wherein: (a) the heavy chain constant region comprises the amino acid sequence set forth in SEQ ID NO: 82, SEQ ID NO: 83, SEQ ID NO: 84, or SEQ ID NO: 85; and/or (b) the light chain constant region comprises the amino acid sequence set forth in SEQ ID NO: 86.

Clause 17. The anti-CD3 antibody or antigen-binding fragment thereof of any one of clauses 1-16, wherein the antibody comprises a human variable region framework region.

Clause 18. The anti-CD3 antibody or antigen-binding fragment thereof of any one of clauses 1-16, which is a fully human or an antigen-binding fragment thereof.

Clause 19. The anti-CD3 antibody or antigen-binding fragment thereof of any one of clauses 1-16, which is a chimeric antibody or an antigen-binding fragment thereof.

Clause 20. The anti-CD3 antibody or antigen-binding fragment thereof of any one of clauses 1-16, which is a humanized antibody or an antigen-binding fragment thereof.

Clause 21. The anti-CD3 antibody or antigen-binding fragment thereof of any one of clauses 1-20, wherein the antigen-binding fragment is a Fab, Fab′, F(ab′)2, variable fragment (Fv), or single chain variable region (scFv).

Clause 22. The anti-CD3 antibody or antigen-binding fragment thereof of clause 21, wherein the antigen-binding fragment is an scFv.

Clause 23. An antibody or an antigen-binding fragment thereof, which cross-competes for binding to CD3 with an anti-CD3 antibody or an antigen-binding fragment thereof of any one of clauses 1-22.

Clause 24. An antibody or an antigen-binding fragment thereof, which binds to the same epitope region on CD3 with an anti-CD3 antibody or an antigen-binding fragment thereof of any one of clauses 1-22.

Clause 25. A composition comprising the anti-CD3 antibody or antigen-binding fragment thereof of any one of clauses 1-24.

Clause 26. The composition of clause 25, which is a pharmaceutical composition that further comprises a pharmaceutically acceptable carrier.

Clause 27. An immunoconjugate comprising the anti-CD3 antibody or antigen-binding fragment thereof of any one of clauses 1-24, linked to a therapeutic agent.

Clause 28. The immunoconjugate of clause 27, wherein the therapeutic agent is a drug, a cytotoxin, or a radioactive isotope.

Clause 29. A composition comprising the immunoconjugate of clause 27 or 28.

Clause 30. The composition of clause 29, which is a pharmaceutical composition that further comprises a pharmaceutically acceptable carrier.

Clause 31. A multi-specific molecule comprising the antibody or antigen-binding fragment thereof of any one of clauses 1-24, linked to one or more functional moieties.

Clause 32. The multi-specific molecule of clause 31, wherein the one or more functional moieties have a different binding specificity than the antibody or antigen binding fragment thereof.

Clause 33. A multi-specific molecule comprising a first binding specificity for CD3 and a second binding specificity.

Clause 34. The multi-specific molecule of clause 33, wherein the first binding specificity comprises the antibody or antigen-binding fragment thereof of any one of clauses 1-24.

Clause 35. The multi-specific molecule of clause 33, wherein the second binding specificity comprises an anti-CD33 antibody or antigen-binding fragment thereof.

Clause 36. The multi-specific molecule of any one of clauses 33-35, wherein the second binding specificity comprises a V_{H} comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a V_{L} comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92.

Clause 37. The multi-specific molecule of any one of clauses 33-36, wherein the second binding specificity comprises a VH comprising the amino acid sequence set forth in SEQ ID NO: 93, and a VL comprising the amino acid sequence set forth in SEQ ID NO: 94.

Clause 38. The multi-specific molecule of clause 33, wherein the second binding specificity comprises an anti-U5 snRNP200 antibody or antigen-binding fragment thereof.

Clause 39. The multi-specific molecule of any one of clauses 33, 34, and 38, wherein the second binding specificity comprises a VH comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a VL comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121.

Clause 40. The multi-specific molecule of any one of clauses 33, 34, and 38-39, wherein the second binding specificity comprises a VH comprising the amino acid sequence set forth in SEQ ID NO: 122, and a VL comprising the amino acid sequence set forth in SEQ ID NO: 123.

Clause 41. The multi-specific molecule of any one of clauses 33, 34, and 38-40, wherein the multi-specific molecule comprises the amino acid sequence set forth in SEQ ID NO: 128 or SEQ ID NO: 130.

Clause 42. The multi-specific molecule of clause 33, wherein the second binding specificity comprises an anti-CD371 antibody or antigen-binding fragment thereof.

Clause 43. The multi-specific molecule of any one of clauses 33, 34, and 42, wherein the second binding specificity comprises a VH comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134; and a VL comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137.

Clause 44. The multi-specific molecule of any one of clauses 33, 34, and 42-43, wherein the second binding specificity comprises a VH comprising the amino acid sequence set forth in SEQ ID NO: 138, and a VL comprising the amino acid sequence set forth in SEQ ID NO: 139.

Clause 45. The multi-specific molecule of any one of clauses 33, 34, and 42-44, wherein the multi-specific molecule comprises the amino acid sequence set forth in SEQ ID NO: 144.

Clause 46. A composition comprising the multi-specific molecule of any one of clauses 33-45.

Clause 47. The composition of clause 46, which is a pharmaceutical composition that further comprises a pharmaceutically acceptable carrier.

Clause 48. An immunocytokine comprising an antibody or antigen-binding fragment thereof of any one of clauses 1-24 and an IL-18 polypeptide or a fragment thereof.

Clause 49. The immunocytokine of clause 48, wherein the IL-18 polypeptide comprises the amino acid sequence set forth in SEQ ID NO: 71.

Clause 50. An immunocytokine comprising a multispecific molecule of any one of clauses 33-45 and an IL-18 polypeptide or a fragment thereof.

Clause 51. The immunocytokine of clause 50, wherein the IL-18 polypeptide comprises the amino acid sequence set forth in SEQ ID NO: 71.

Clause 52. A composition comprising the immunocytokine of any one of clauses 47-51.

Clause 53. The composition of clause 52, which is a pharmaceutical composition that further comprises a pharmaceutically acceptable carrier.

Clause 54. A nucleic acid that encodes an antibody or antigen-binding fragment thereof of any one of clauses 1-24.

Clause 55. A nucleic acid that encodes a multispecific molecule of any one of clauses 33-45.

Clause 56. A nucleic acid that encodes an immunocytokine of any one of clauses 47-51.

Clause 57. A vector comprising the nucleic acid of any one of clauses 54-56.

Clause 58. A host cell comprising the vector of clause 57.

Clause 59. An immunoresponsive cell comprising the antibody or antigen-binding fragment thereof of any one of clauses 1-24, the immunoconjugate of clause 27 or 28, the multi-specific molecule of any one of clauses 33-45, the immunocytokine of any one of clauses 47-51, the nucleic acid of any one of clauses 54-56, or the vector of clause 57.

Clause 60. The immunoresponsive cell of clause 59, further comprising antigen-recognizing receptor.

Clause 61. The immunoresponsive cell of clause 60, wherein the antigen-recognizing receptor is a recombinant T cell receptor (TCR), a chimeric antigen receptor (CAR), or a TCR like fusion molecule.

Clause 62. The immunoresponsive cell of clause 60 or 61, wherein the antigen-recognizing receptor is a CAR.

Clause 63. The immunoresponsive cell of any one of clauses 59-62, wherein the antigen is a tumor antigen.

Clause 64. The immunoresponsive cell of clause 63, wherein the tumor antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD371, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGFIR, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PRI), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, U5 snRNP200, and ZDHHC11.

Clause 65. The immunoresponsive cell of any one of clauses 59-64, wherein the immunoresponsive cell is a cell of the lymphoid lineage or a cell of the myeloid lineage.

Clause 66. The immunoresponsive cell of any one of clauses 59-65, wherein the cell is selected from the group consisting of a T cell, a Natural Killer (NK) cell, a B cell, a monocyte, and a macrophage, a pluripotent stem cell from which a lymphoid cell may be differentiated, a pluripotent stem cell from which a myeloid cell may be differentiated, and combinations thereof.

Clause 67. The immunoresponsive cell of any one of clauses 59-66, wherein the cell is a T cell.

Clause 68. The immunoresponsive cell of any one of clauses 59-67, wherein the cell is a Natural Killer (NK) cell.

Clause 69. The immunoresponsive cell of any one of clauses 59-68, wherein the cell is autologous.

Clause 70. The immunoresponsive cell of any one of clauses 59-68, wherein the cell is allogeneic.

Clause 71. A composition comprising the immunoresponsive cell of any one of clauses 59-70.

Clause 72. The composition of clause 71, which is a pharmaceutical composition further comprising a pharmaceutically acceptable excipient.

Clause 73. A method for detecting CD3 in a whole cell, a tissue, or a blood sample, comprising: (a) contacting a cell, tissue or blood sample with the antibody or antigen-binding fragment thereof of any one of clauses 1-24, wherein the antibody or antigen-binding fragment thereof comprises a detectable label; and (b) determining the amount of the labeled antibody or antigen-binding fragment thereof bound to the cell, tissue or blood sample by measuring the amount of detectable label associated with said cell or tissue, wherein the amount of bound antibody or antigen-binding fragment thereof indicates the amount of CD3 in the cell, tissue or blood sample.

Clause 74. A method of treating or ameliorating a disease or disorder in a subject, comprising administering to the subject the antibody or antigen-binding fragment thereof of any one of clauses 1-24, the immunoconjugate of clause 27 or 28, the multi-specific molecule of any one of clauses 33-45, the immunocytokine of any one of clauses 47-51, the cell of any one of clauses 59-70, or the composition of any one of clauses 25, 26, 29, 30, 46, 47, 52, 53, 71, and 72.

Clause 75. The method of clause 74, wherein the disease or disorder is a tumor.

Clause 76. A method of reducing tumor burden in a subject, comprising administering to the subject an antibody or antigen-binding fragment thereof of any one of clauses 1-24, the immunoconjugate of clause 27 or 28, the multi-specific molecule of any one of clauses 33-45, the immunocytokine of any one of clauses 47-51, the cell of any one of clauses 59-70, or the composition of any one of clauses 25, 26, 29, 30, 46, 47, 52, 53, 71, and 72.

Clause 77. The method of clause 76, wherein the method reduces the number of the tumor cells, reduces the tumor size, and/or eradicates the tumor in the subject.

Clause 78. A method of treating and/or preventing a tumor in a subject, comprising administering to the subject an antibody or antigen-binding fragment thereof of any one of clauses 1-24, the immunoconjugate of clause 27 or 28, the multi-specific molecule of any one of clauses 33-45, the immunocytokine of any one of clauses 47-51, the cell of any one of clauses 59-70, or the composition of any one of clauses 25, 26, 29, 30, 46, 47, 52, 53, 71, and 72.

Clause 79. A method of increasing or lengthening survival of a subject having a tumor, comprising administering to the subject an antibody or antigen-binding fragment thereof of any one of clauses 1-24, the immunoconjugate of clause 27 or 28, the multi-specific molecule of any one of clauses 33-45, the immunocytokine of any one of clauses 47-51, the cell of any one of clauses 59-70, or the composition of any one of clauses 25, 26, 29, 30, 46, 47, 52, 53, 71, and 72.

Clause 80. The method of clause 79, wherein the method reduces or eradicates tumor burden in the subject.

Clause 81. The method of any one of clauses 74-80, wherein the tumor is cancer.

Clause 82. The method of any one of clauses 74-81, wherein the tumor is hematological cancer or solid tissue cancer.

Clause 83. The method of clause 82, wherein the hematological cancer is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms.

Clause 84. The method of clause 83, wherein the hematological cancer is acute myeloid leukemia (AML).

Clause 85. A method of inducing myeloid depletion in a subject in need thereof, comprising administering to the subject the multi-specific molecule of any one of clauses 33-45 or the composition of clause 46 or 47.

Clause 86. The method of any one of clauses 74-85, wherein the subject is a human.

Clause 87. A kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof of any one of clauses 1-24, the immunoconjugate of clause 27 or 28, the multi-specific molecule of any one of clauses 33-45, the immunocytokine of any one of clauses 47-51, the cell of any one of clauses 59-70, or the composition of any one of clauses 25, 26, 29, 30, 46, 47, 52, 53, 71, and 72.

Clause 88. The kit of clause 87, wherein the kit further comprises written instructions for using the antibody or antigen-binding fragment thereof, immunoconjugate, multi-specific molecule, or composition for treating or ameliorating a disease or disorder in a subject, treating or ameliorating a disease or disorder in a subject, reducing tumor burden in a subject, treating and/or preventing a tumor in a subject, and/or increasing or lengthening survival of a subject having a tumor.

### EXAMPLES

The following examples are put forth so as to provide those of ordinary skill in the art with a complete disclosure and description of how to make and use the antibodies, make and use immunoconjugates of such antibodies, compositions comprising thereof, screening, and therapeutic methods of the presently disclosed subject matter, and are not intended to limit the scope of what the inventors regard as their presently disclosed subject matter. It is understood that various other embodiments can be practiced, given the general description provided above.

### Example 1

Although autologous chimeric antigen receptor (CAR) T cells have revolutionized the treatment of malignancies, this therapy is plagued by high production costs, delays in treatment due to manufacturing length/delays, and limited capacity for redosing due to immune rejection. To circumvent these issues, allogeneic CAR T cells have been deployed, but these have yet to demonstrate significant efficacy, likely due to their rapid immune rejection by the host given their allogeneic nature. Multispecific molecules (e.g., multispecific molecules) are off-the-shelf and utilize a patient's own T cells to eliminate tumors, and hence, are free from immune rejection. In the last few years, multispecific molecules have emerged as a potent form of immunotherapy. Given this early success, it is unclear if these off-the-shelf molecules will compete or complement autologous CAR T cell therapy.

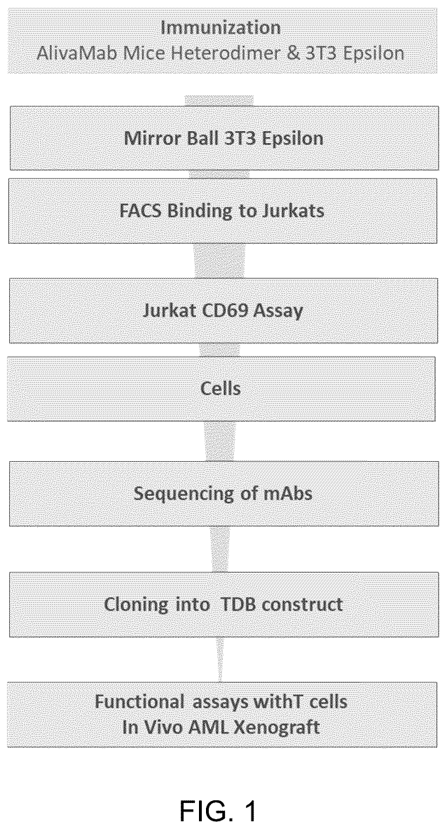

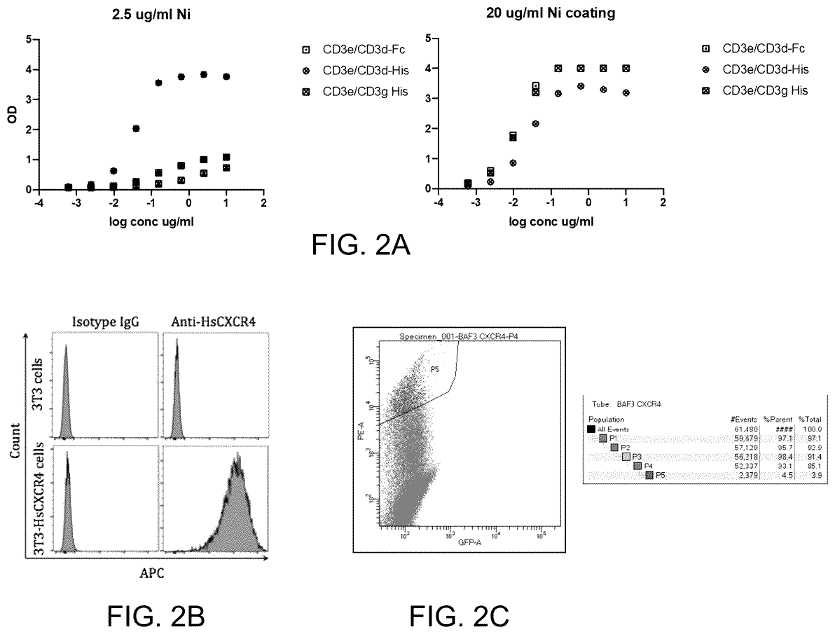

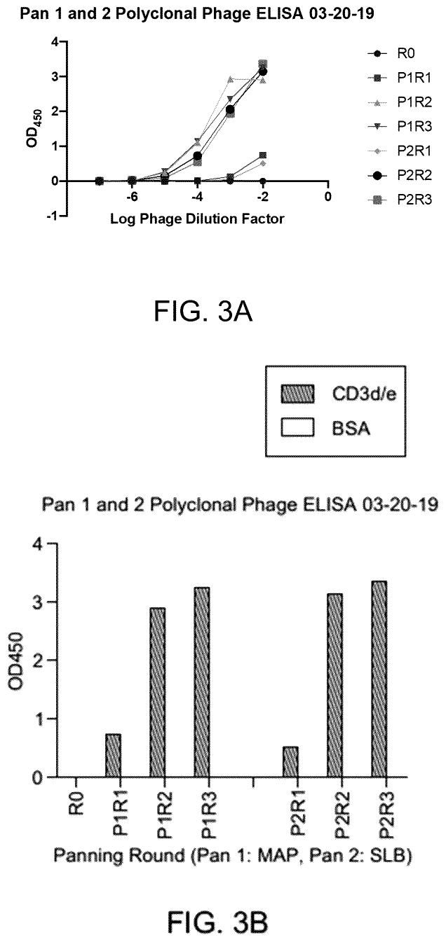

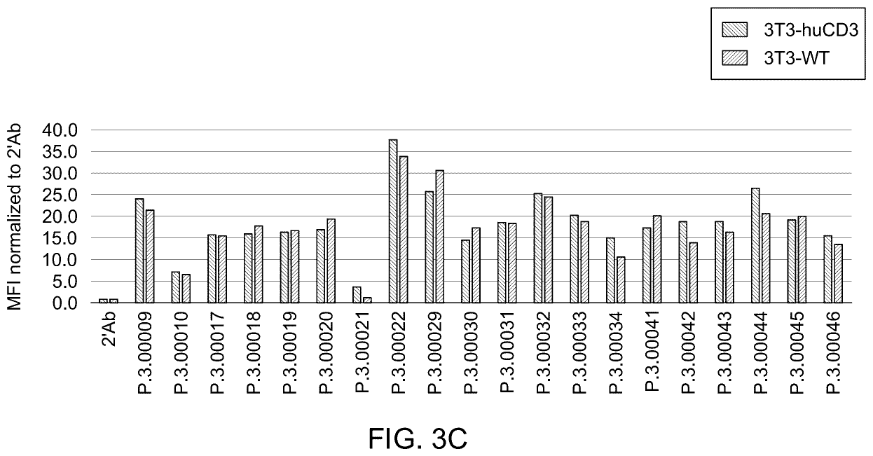

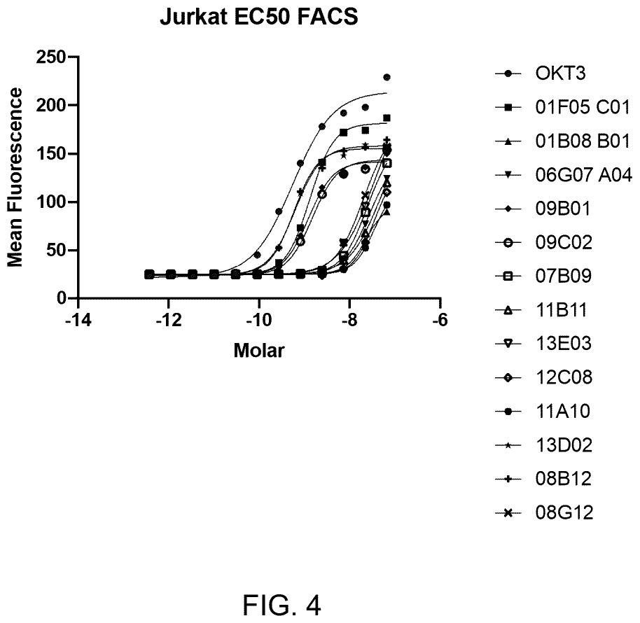

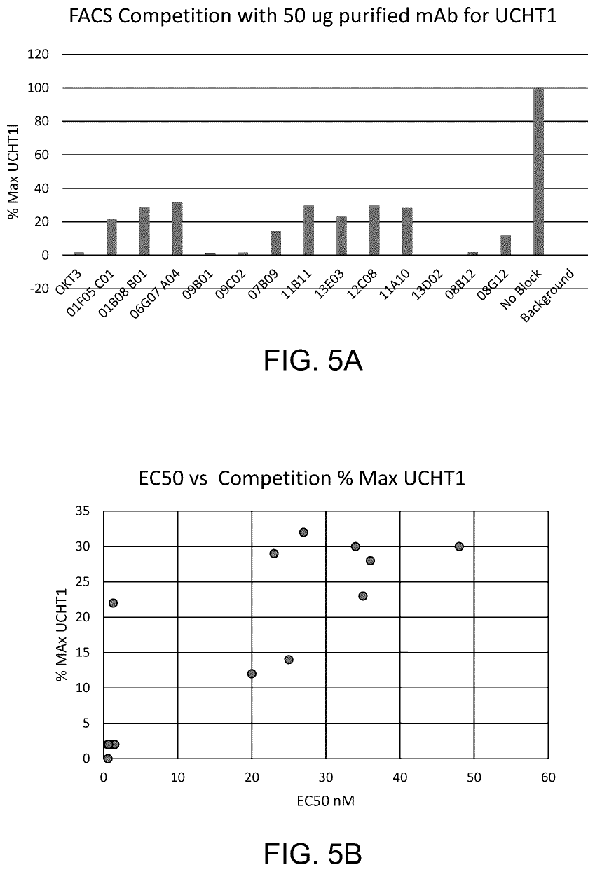

To start, a multistep screening approach was used to generate a panel of 13 purified antibodies that bind to CD3ε (FIG. 1). The immunogens and developed antibodies (e.g., via immunization or phage display) were then analyzed for quality control purposes (FIGS. 2A-3C). Binding affinity was assessed by FACS analysis in Jurkat cells. The developed antibodies showed a wide EC50 range spanning from 1 nM to 50 nM. Notably, all developed antibodies were less potent than OKT3 (FIG. 4). Next, competition assays were run. As shown in FIGS. 5A and 5B, each antibody competed with UCHT1 (an anti-CD3ε antibody) indicating that the antibodies recognize an overlapping epitope on CD3ε and not CD3δ.

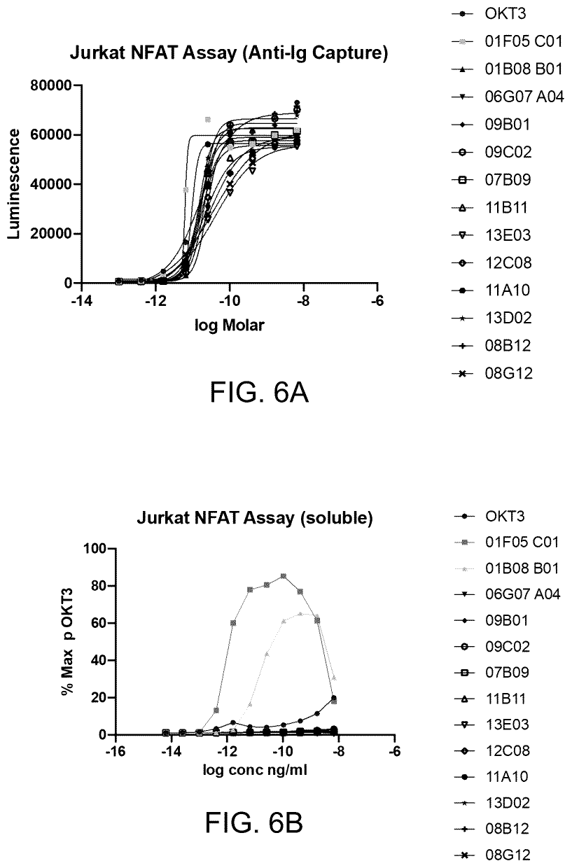

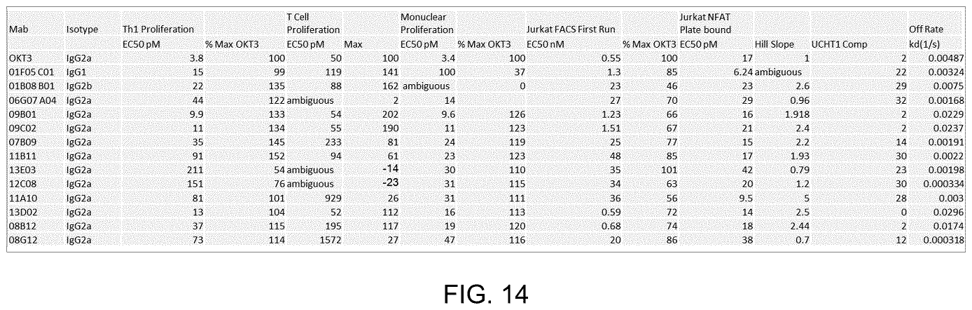

Next, the functional ability to activate T cells was tested. All tested antibodies showed similar T cell activation in Jurkat NFAT assay using plate bound-CD3ε (FIGS. 6A and 6B) in order to different target densities on tumor cells. Notably, two antibodies (01F05 and 01B08) that were strong agonists in Jurkat cells were not IgG2a (FIG. 6B and FIG. 14).

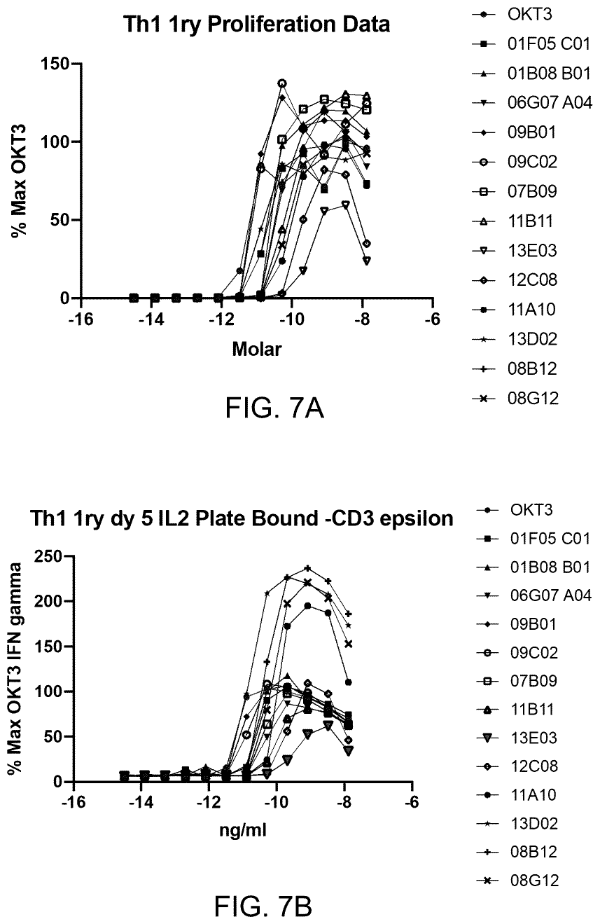

In order to further characterize the presently disclosed antibodies, Th1 proliferation assays were performed. In absence of CD28, functional separation of the antibody panel was observed, which is reflective of the target affinity (FIG. 7A). Further separation was observed using IL2 plate bound-CD3E, which found that 13E03 and 12C08 had the lowest functional effect (FIG. 7B).

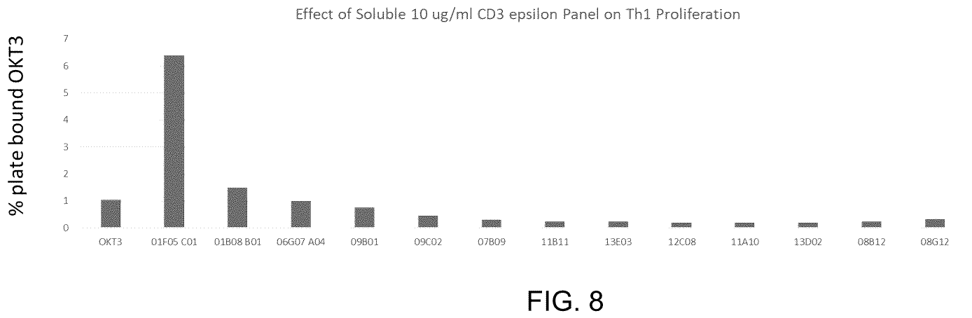

Finally, the effect of soluble CD3ε on Th 1 proliferation was further tested. As shown in FIG. 8, antibodies 01F05, 01B08, 06G07, 09B01, and the OKT3 control had low agonistic activity on primary cells as well as Jurkat cells but at a much-reduced potency.

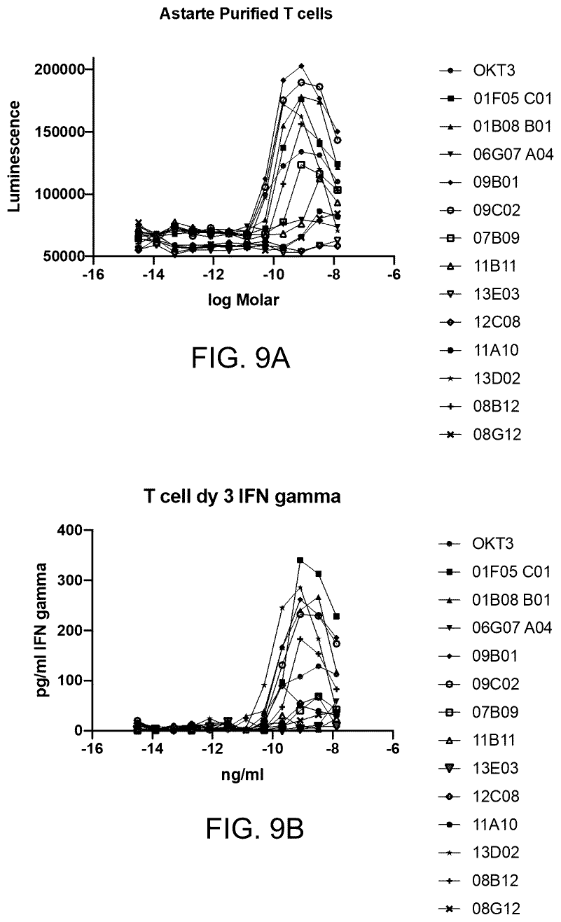

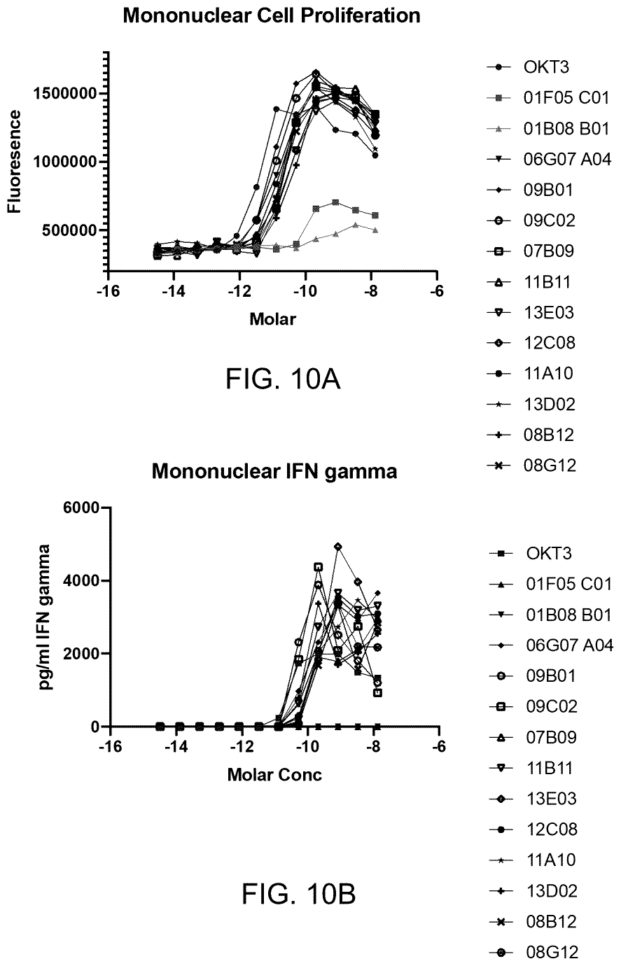

Further analysis were conducted to determine the functional properties of the antibodies. A similar functional EC50 ranking was seen using purified T cells including resting naïve and memory T cells expressing CD28) but with less potency (FIGS. 9A and 9B). Next, a mononuclear functional assay was performed. As seen in FIGS. 10A and 10B, the proliferation and activation of T cells were influenced by the costimulatory effects of monocytes, NK cells, and B cells and by costimulatory signals such as CD80, CD86, CD40L, and ICOSL.

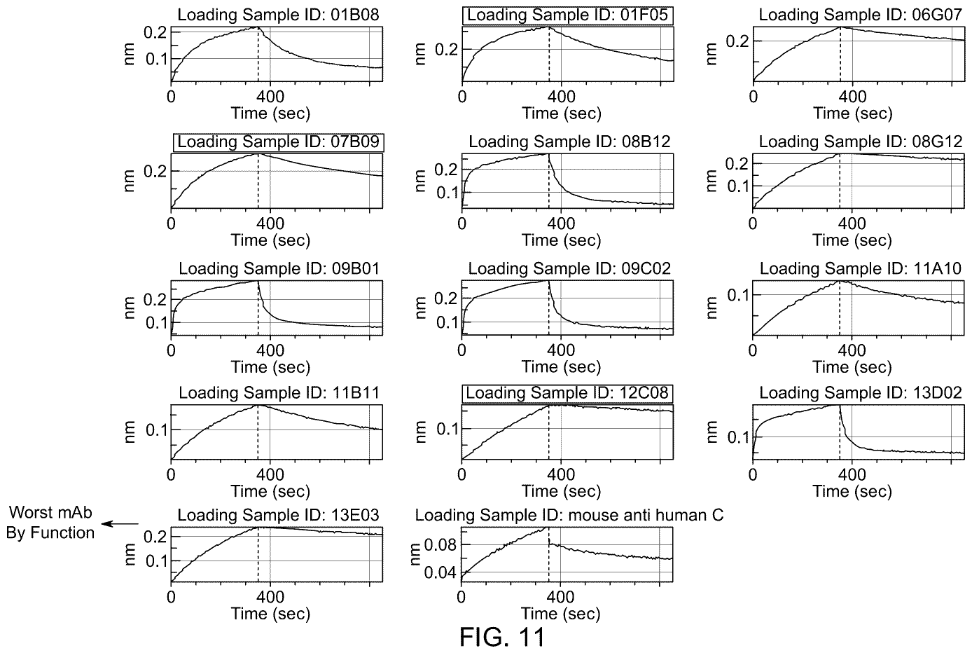

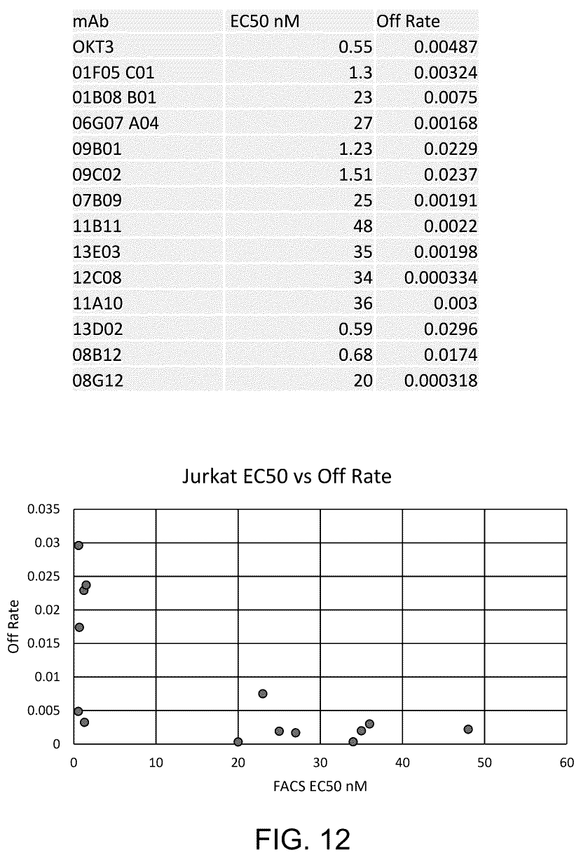

The binding affinity was further characterized by the off-rate analysis (FIG. 11). The off-rate analysis did not correlate to the binding affinity calculated from the EC50 analysis in Jurkat cells, indicating that the structure of the CD3ε/δ heterodimer is not the same as the cell membrane-bound CD3ε (FIG. 12).

Overall these data indicate that several factors can modulate the potency of an anti-CD3 antibody including the cell type (e.g., effector vs resting). Indeed, fitting MHC-I peptide presentation models, the more the T cell is in an activated state, the less CD3ε stimulation is required. In addition, based on FACS vs Th1 data, only a fraction of the CD3ε on the cell surface is required to be crosslinked to fully activate a T cell. Finally, it was noted that increased costimulation can shift the number of CD3ε that needs to be engaged.

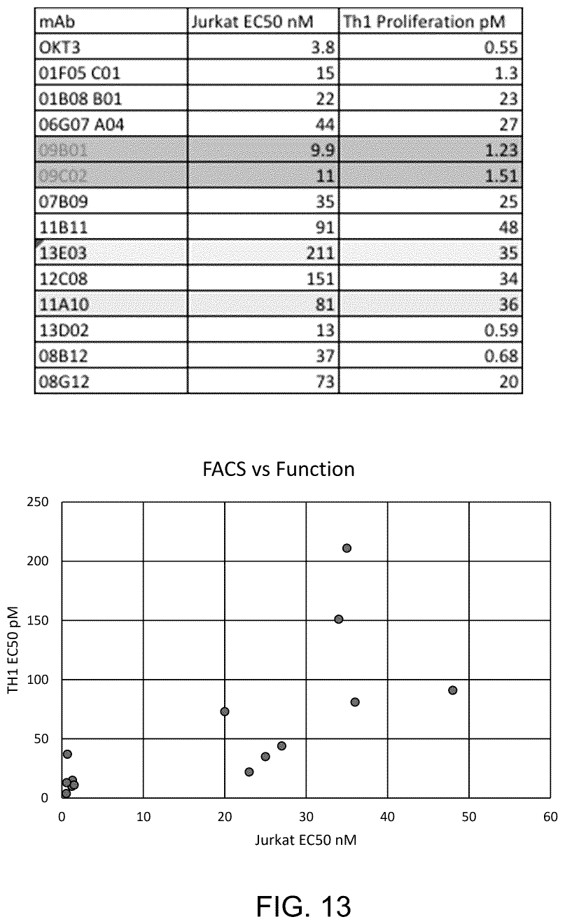

Next, the results of the Jurkat EC₅₀ analysis by FACS and the Th1 cell proliferation assay were utilized in the selection of high-, intermediate-,/and low-affinity antibodies against CD3ε (FIG. 13). A summary of the binding affinities and functional assays for the developed antibodies is presented in FIG. 14. Based on these criteria, the 12C08, 07B09, 09B01, and 01F05 antibodies were selected for further studies.

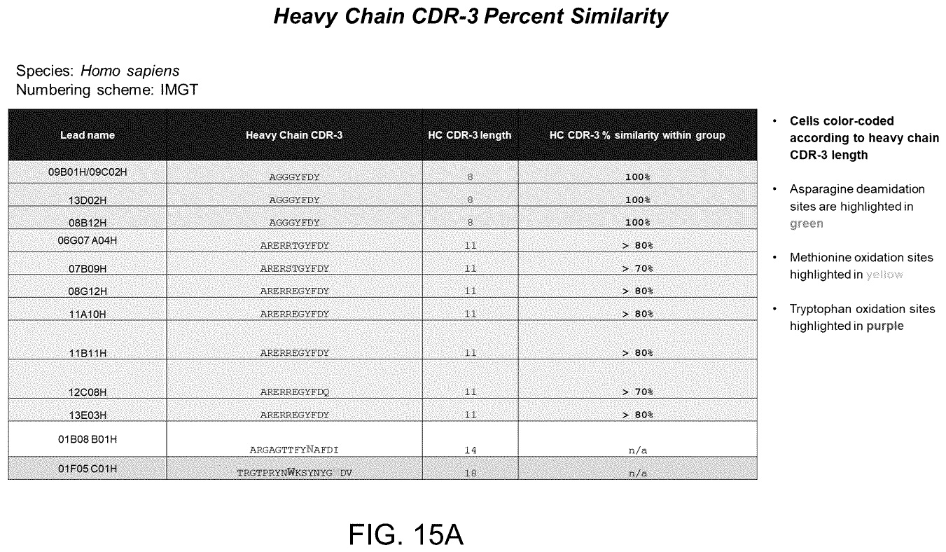

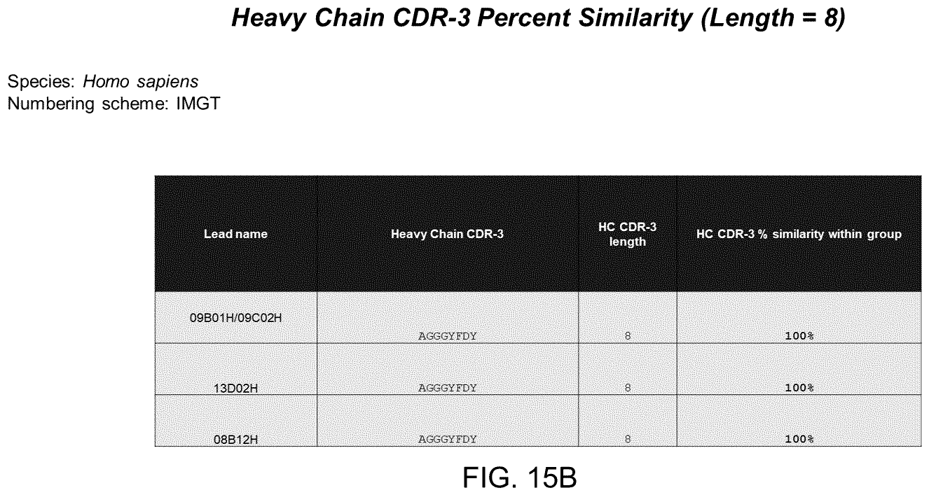

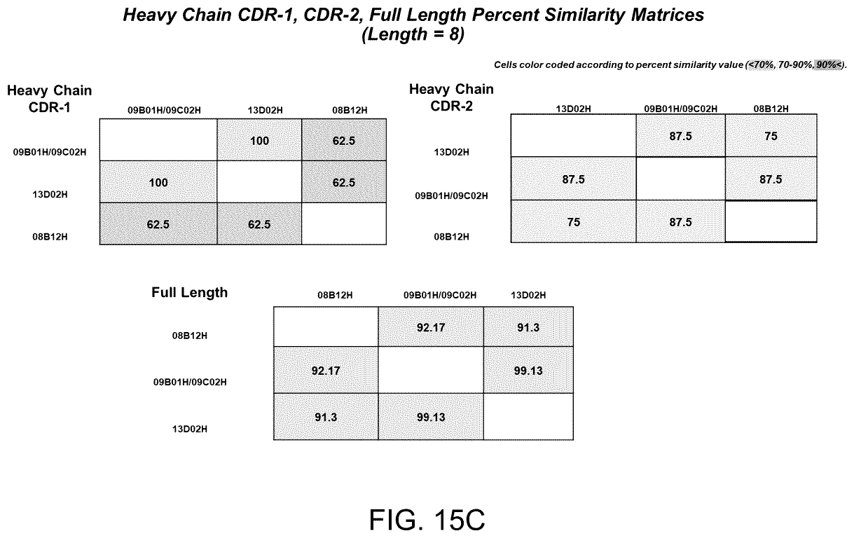

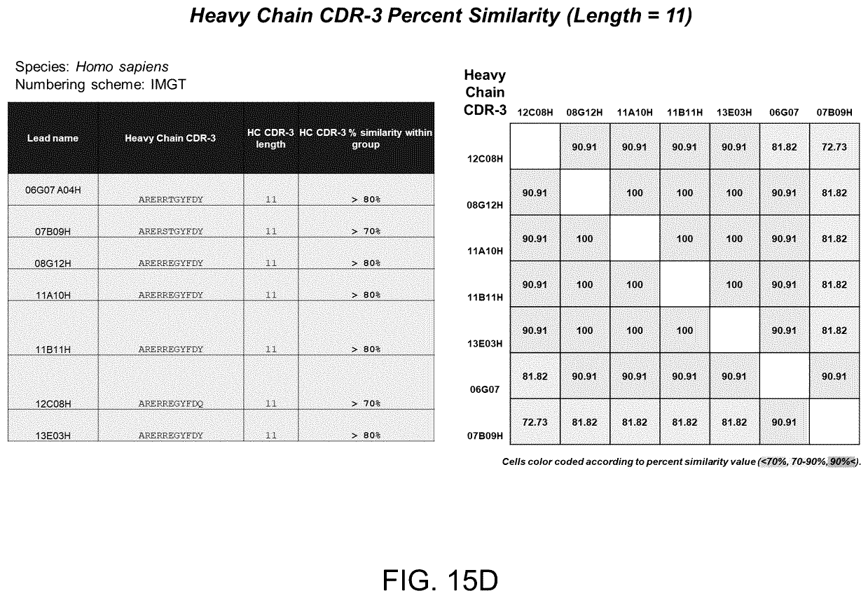

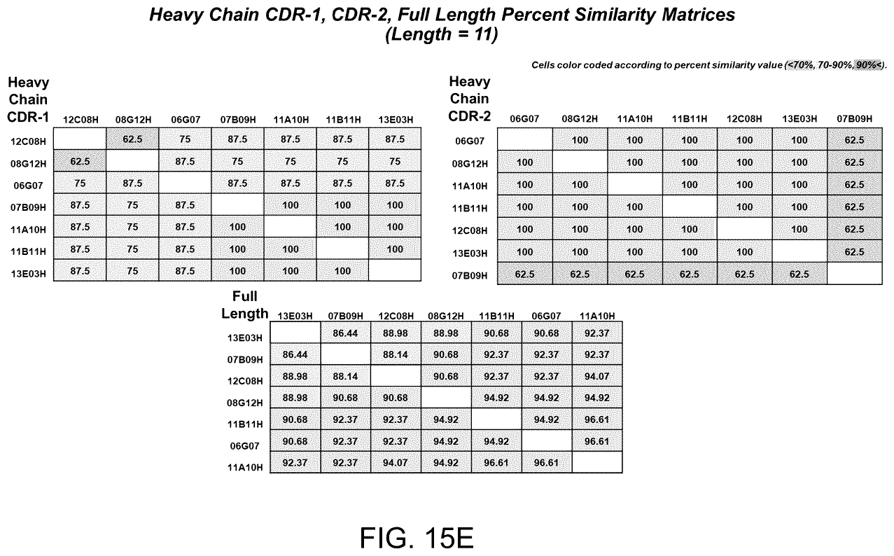

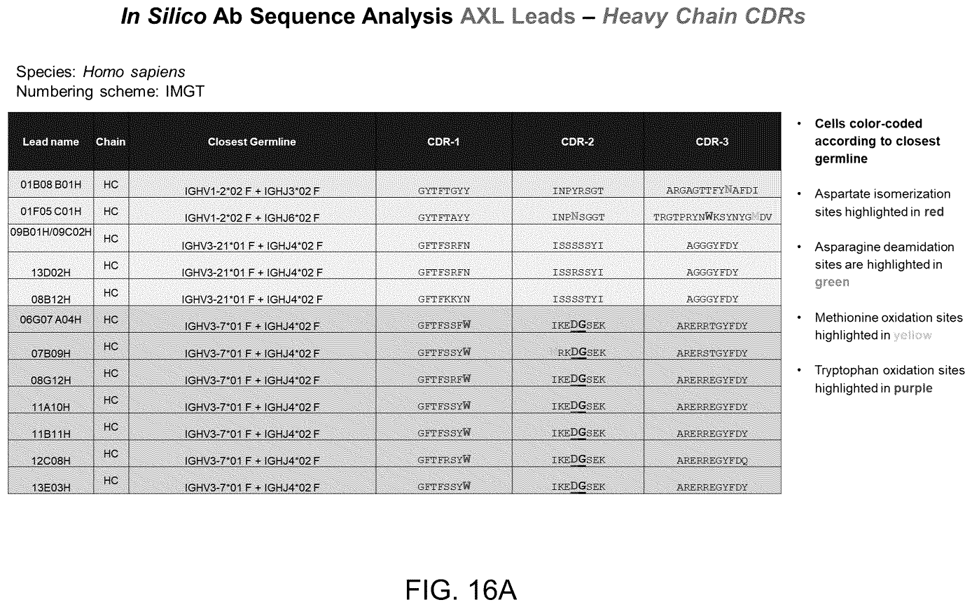

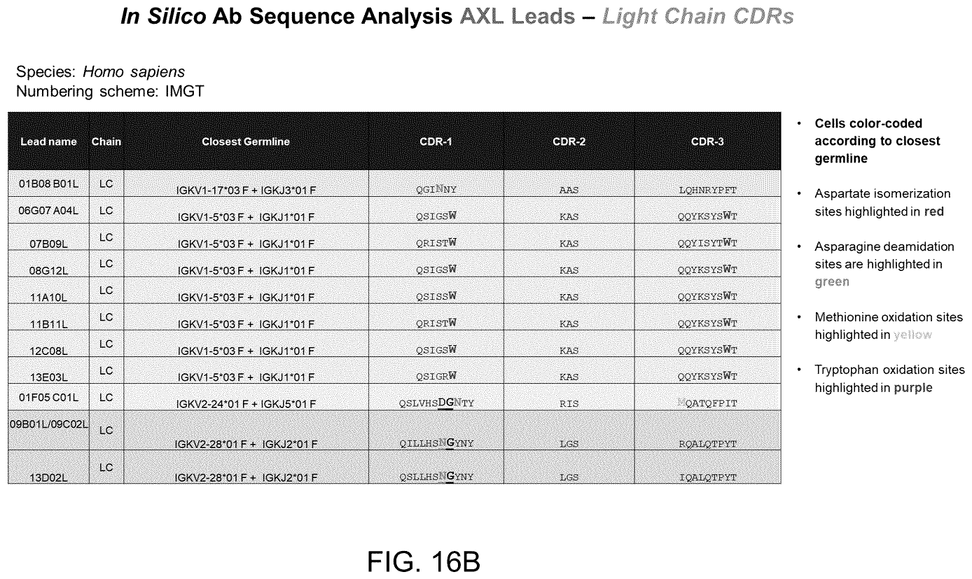

Upon identification of the sequences of each antibody, a sequence comparison analysis was performed on the identified CDR binder regions (FIGS. 15A-15E), and in silico analysis identified sequence liabilities (FIGS. 16A and 16B). Briefly, heavy chain CDR3 sequences were sorted according to length and analyzed for pairwise similarity within the group using Clustal Omega alignment. CDR sequences were also analyzed for the following potential post-translational modification sites:
Aspartate isomerization (DG/DS/DD/DH);
Asparagine deamidation (NG/NS/NH/NT/NN/NA);
N-linked glycosylation (N-X-T/S where X is not P);
Methionine & Tryptophan oxidation (M & W); and
anomalous Cysteines (C).

Overall, it was observed that most of the analyzed sequences had a CDR3 identity similarity greater than 70% within the group. In addition, several potential post-translational modification sites were identified:
06G07 A04H, 07B09H, 08G12H, 11A10H, 11B11H, 12C08H and 13E03H containing DG isomerization hot-spot motifs in the heavy chain CDR2;
01F05 COIL containing a DG isomerization hot-spot motif in the light chain CDR1; and
09B01L, 09C02L, and 13D02L containing NG isomerization hot-spot motifs in the light chain CDR1.

In conclusion, the presently disclosed subject matter developed fully-human antibodies directed against CD38.

### Example 2

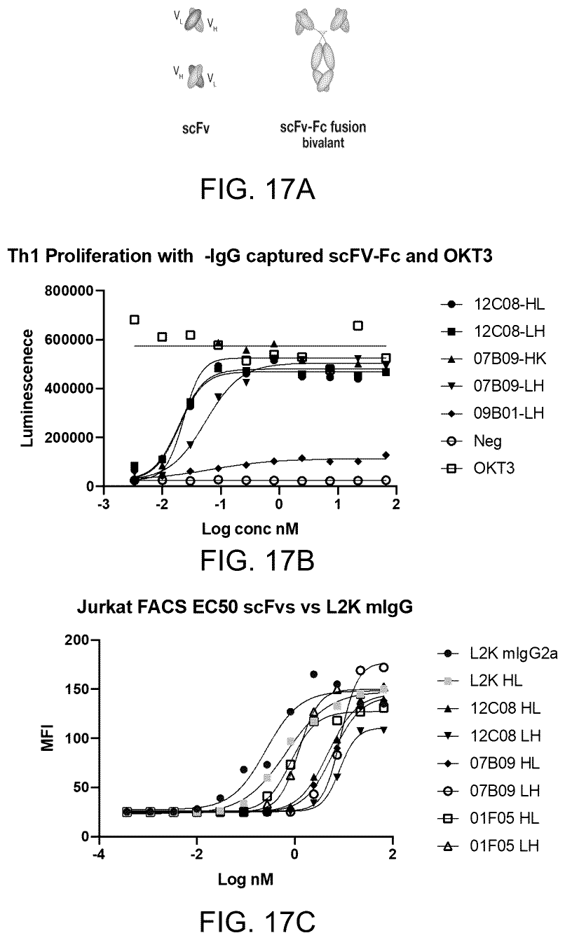

The 12C08, 07B09, and 09B01 scFvs were reformatted as scFv-Fc bivalent fusion molecules (FIG. 17A) and were functionally validated using the Th1 proliferation assay with IgG captured scFv-Fc (FIG. 17B) and by FACS EC₅₀ in Jurkat cells (FIG. 17C).

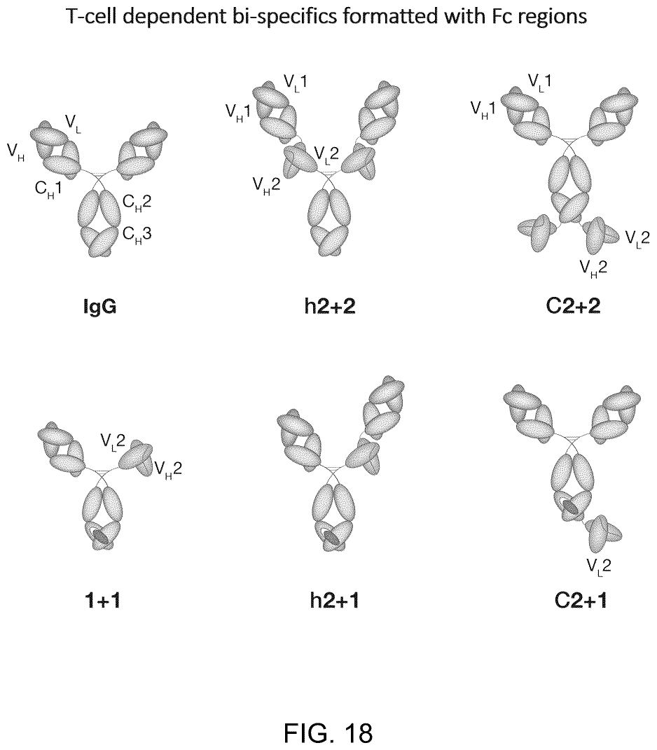

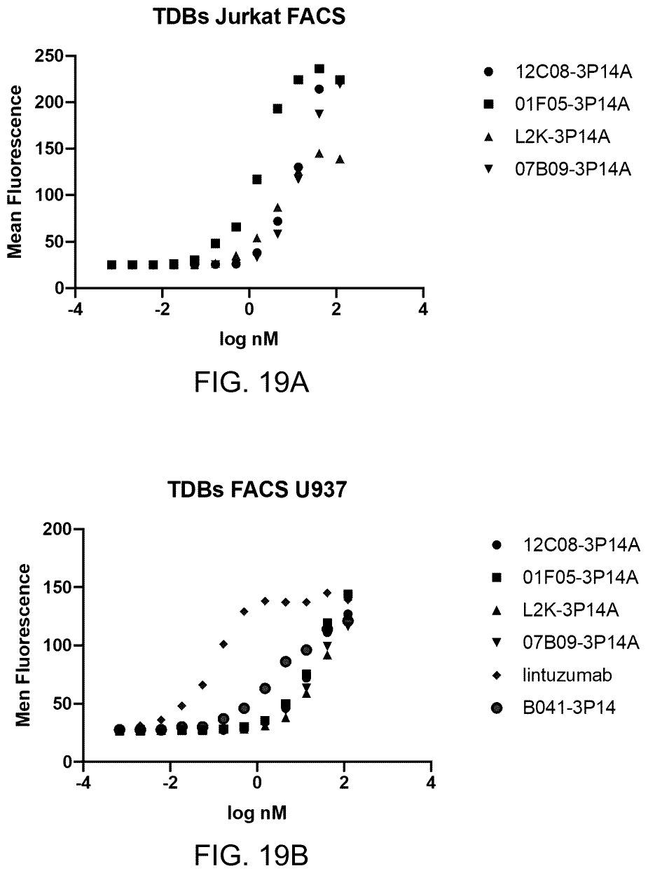

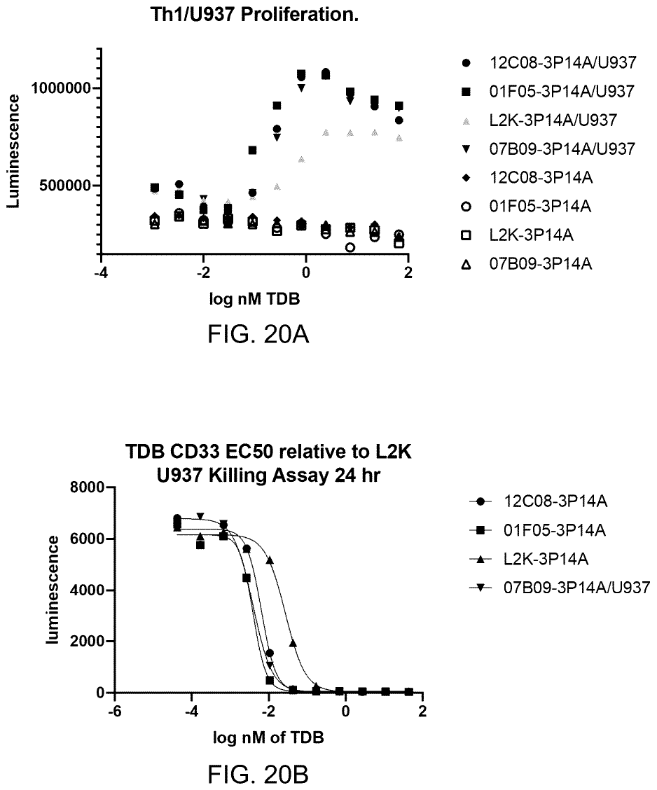

Next, the 12C08, 01F05, and 07B09 antibodies were reformatted into multispecific molecules using the format labeled as “1+1” depicted in FIG. 18. Along with the indicated anti-CD3 antibody or antigen-binding fragment thereof (e.g., 12C08, 01F05, and 07B09 antibodies), each bispecific molecule included an anti-CD33 antibody designated as “3P14” that was previously described in International Patent Application No. PCT/US2022/042448. As seen in FIGS. 19A and 19B, all the multispecific molecules bound to Jurkat cells and U937 cells with varying affinity in comparison to controls. Further, as illustrated in FIGS. 20A and 20B, the multispecific molecules induced increased cell proliferation and cell killing compared to L2K (a CD3 binder used in blinatumomab). Overall these data confirm that the tested anti-CD3 antibodies can be used in the context of bispecific molecules for cancer therapy.

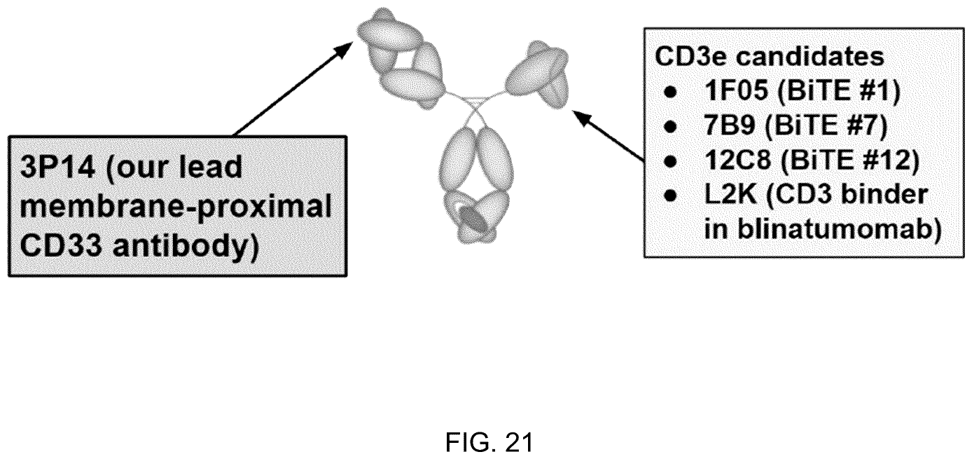

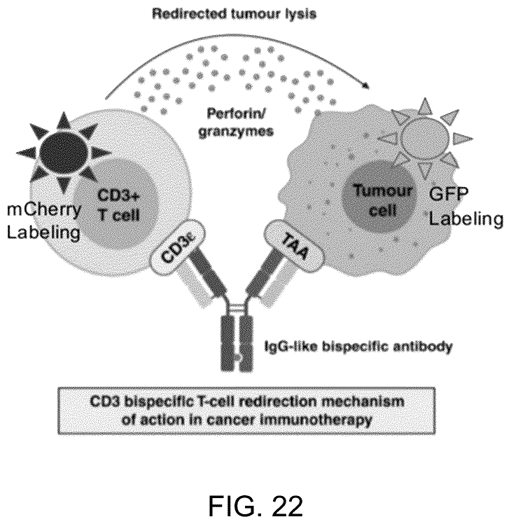

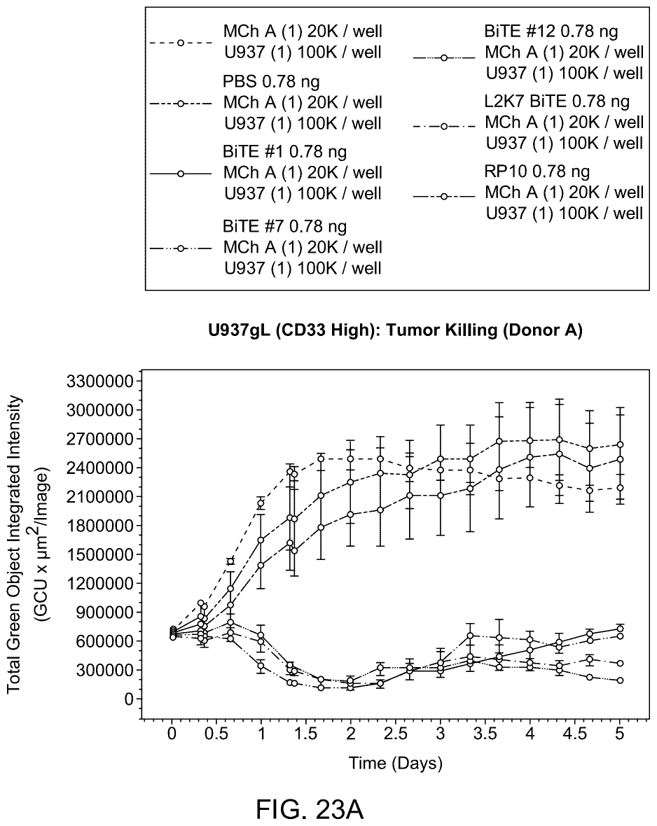

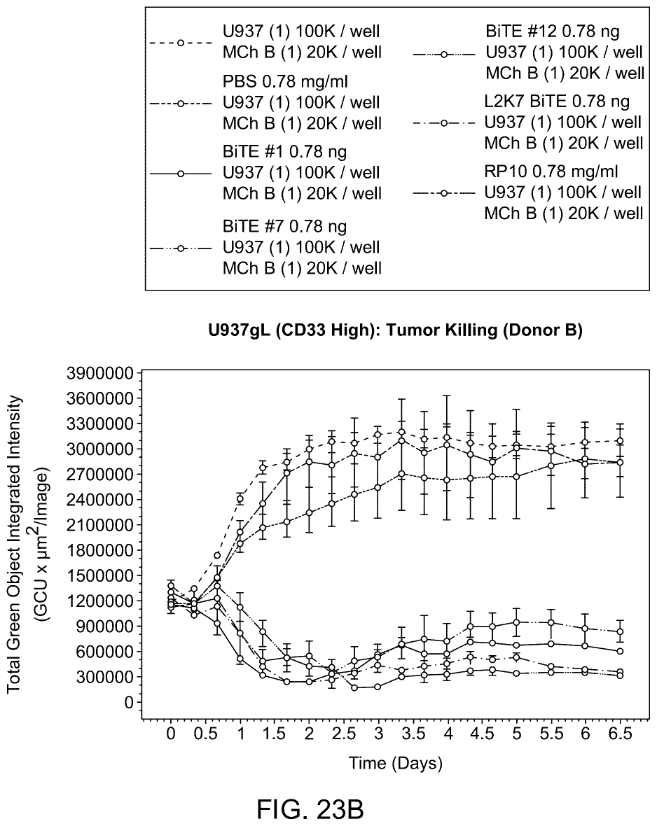

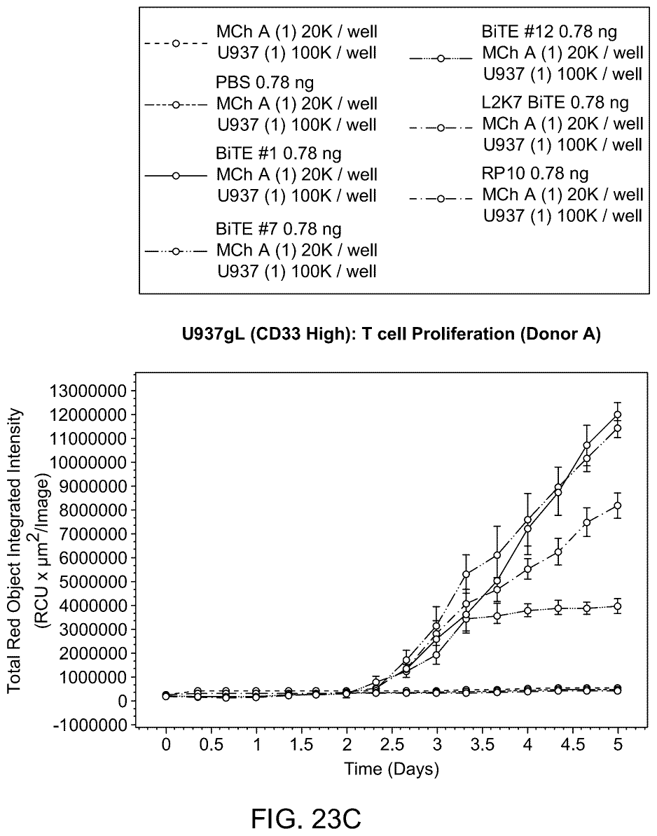

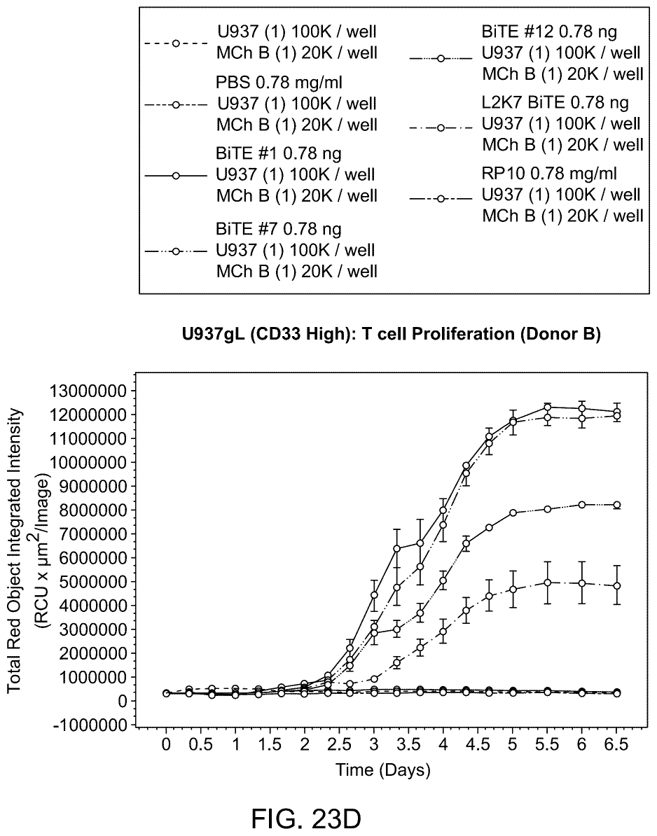

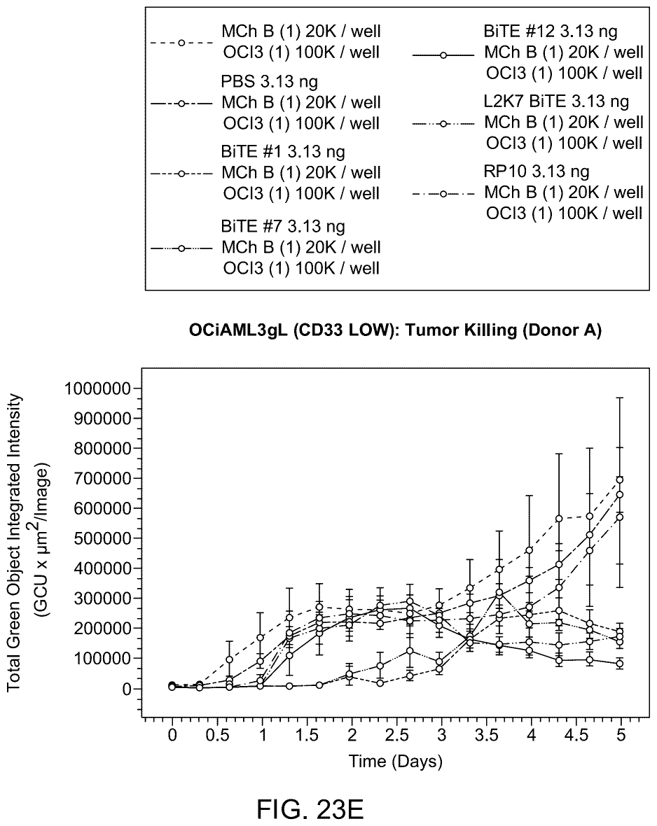

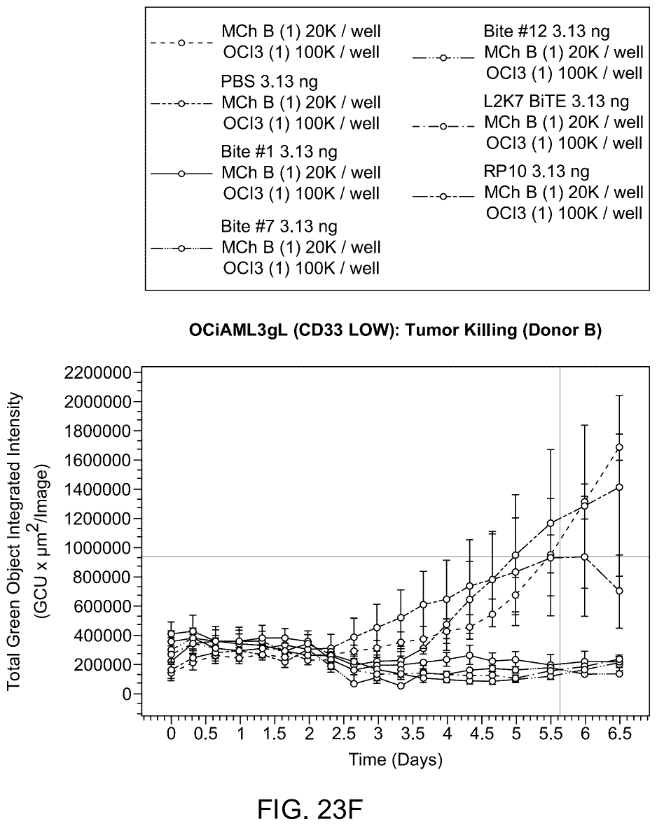

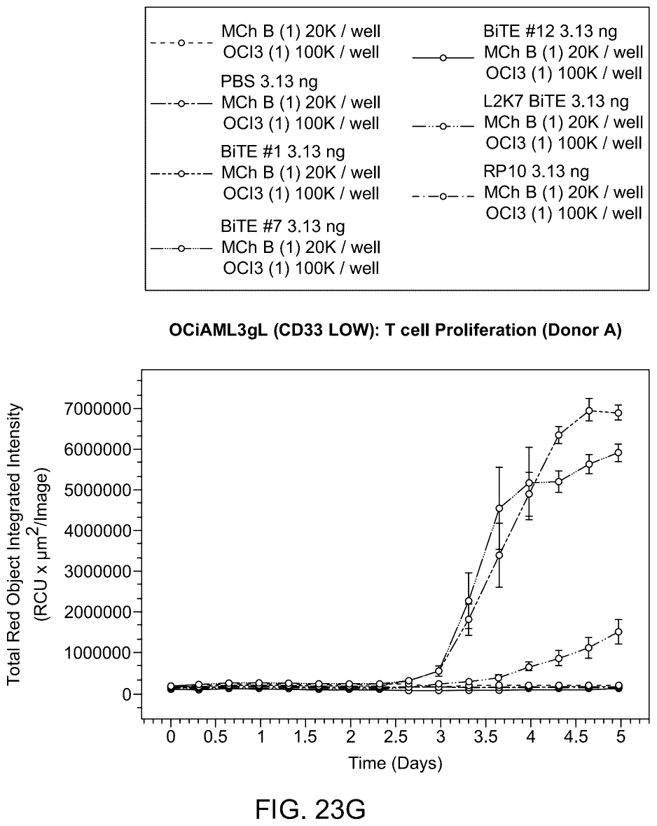

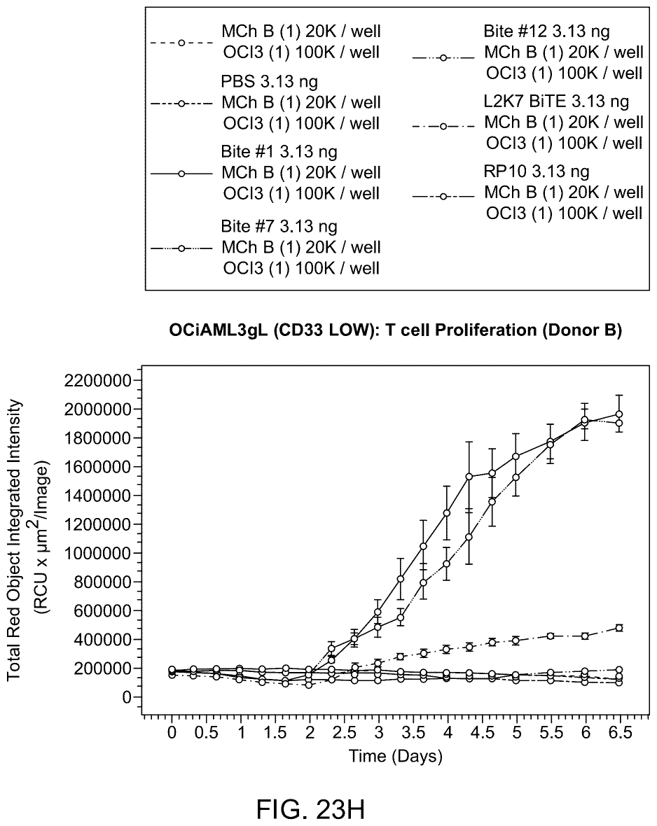

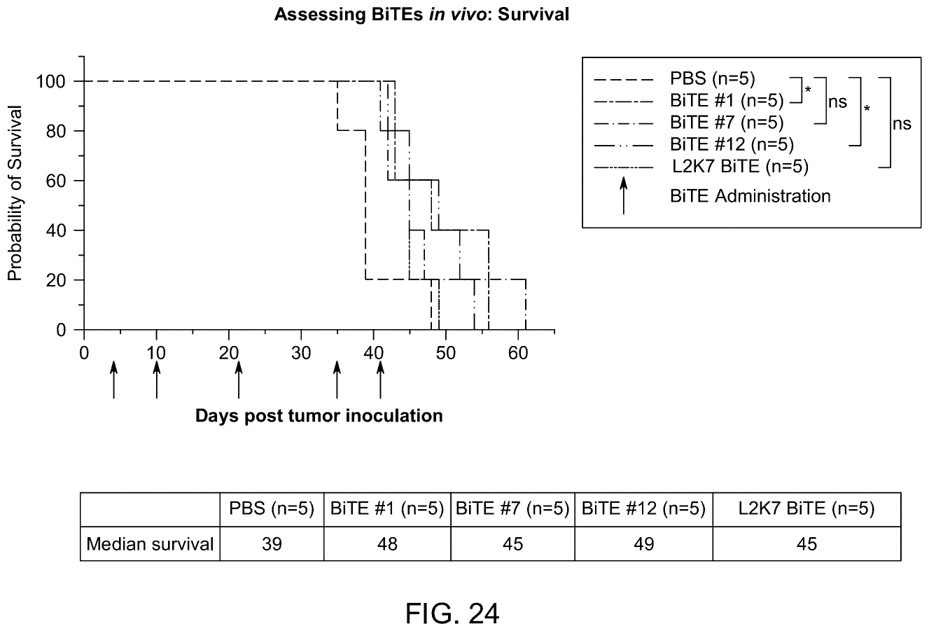

FIG. 21 shows the structure of multispecific molecules integrating a presently disclosed anti-CD3 antibody (1F05, 0709, 12C08) and the anti-CD33 antibody designated “3P14” using a format known as “knob-in-hole” (KIH) which allows for heterodimeric combinations. Three molecules were developed: a) a multispecific molecule including the anti-CD33 3P14 antibody and the anti-CD3 01F05 antibody (also designated as “BiTE #1”); b) a multispecific molecule including the anti-CD33 3P14 antibody and the anti-CD3 07B09 antibody (also designated as “BiTE #7”); and c) a multispecific molecule including the anti-CD33 3P14 antibody and the anti-CD3 12C08 antibody (also designated as “BiTE #12”) To assess the functionality of these multispecific molecules in vitro, the IncuCyte assays were performed using mCherry-expressing T cells and GFP-expressing tumor cells with varying CD33 antigen levels (FIG. 22). While all tested multispecific molecules led to potent tumor eradication, it was observed that BiTE #1 and BiTE #7 led to marked T cell proliferation relative to multispecific molecules developed with L2K7, the contemporary CD3-binder in blinatumomab (FIGS. 23A-23H). Importantly, BiTE #1 and BiTE #12 demonstrated a survival benefit in an AML xenograft model (FIG. 24).

In conclusion, the presently disclosed subject matter demonstrated that these multispecific molecules can elicit potent tumor killing and marked T cell proliferation in vitro. Furthermore, these multispecific molecules demonstrated superior tumor control over those made with the contemporary binder found in the FDA-approved blinatumomab (L2K7), leading to improved survival in xenograft models of acute myeloid leukemia (AML).

### Example 3

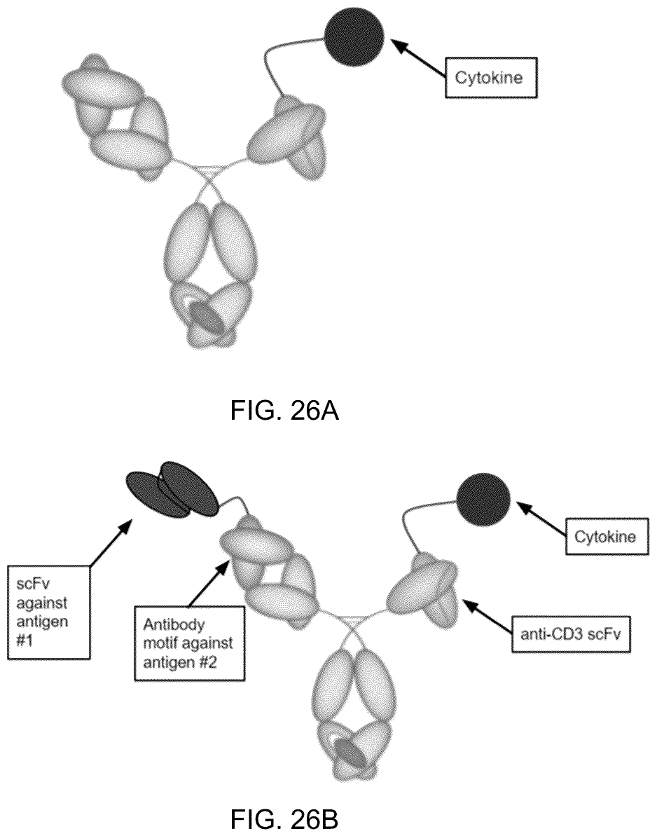

In order to improve tumor killing, the inventors of the presently disclosed subject matter designed improved multispecific molecules including a cytokine. As depicted in FIGS. 26A and 26B, a recombinant cytokine is added to the CD3-binding arm of the multispecific antibody. Cytokines like IL18, IL33, and IL36 gamma can be used since these three cytokines have been shown to markedly improve T cell functionality.

### Example 4

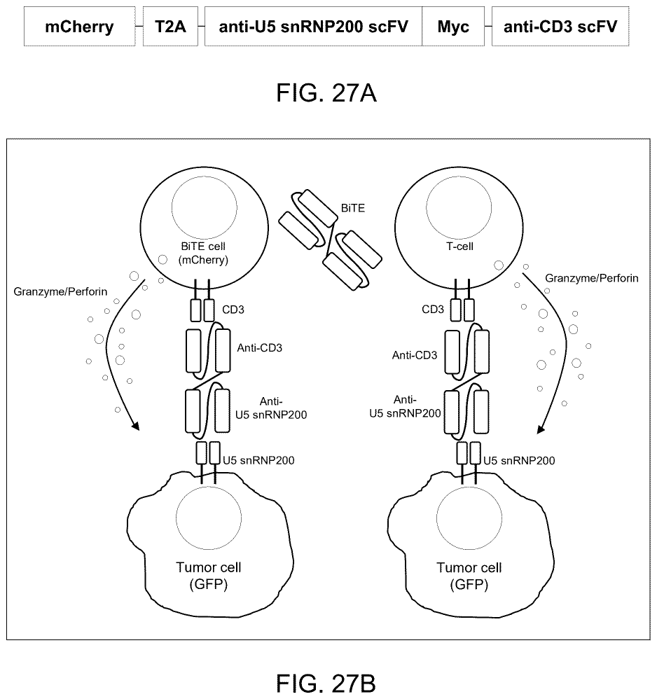

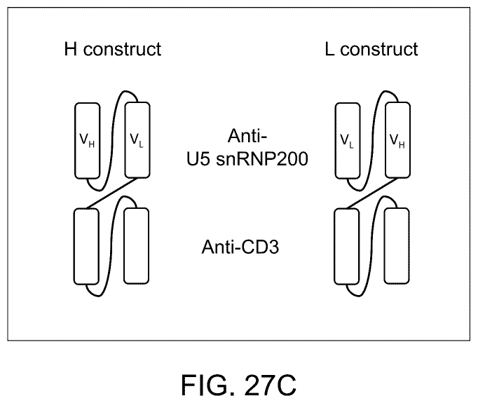

In order to improve tumor killing, the inventors of the presently disclosed subject matter developed T cells expressing multispecific molecules including one of the presently disclosed scFv. As depicted in FIGS. 27A, a bispecific antibody including a first binding specificity for U5 snRNP200 and a second binding specificity for CD3 was developed. The first binding specificity included heavy chain variable region and light chain variable region of antibodies detected in AML patients (Gillissen et al., Blood, The Journal of the American Society of Hematology 131.1 (2018): 131-143), while the second binding specificity included the heavy chain variable region and light chain variable region of the 01F05C01 scFv described in Section 3.1. As seen in FIG. 27B, these bispecific antibodies allowed activation and cytotoxic effects induced by T cells secreting them as well as bystanding T cells. See FIG. 27B.

To test these bispecific antibodies, multiple constructs were prepared. The H construct included the first binding specificity for U5 snRNP200 having the variable regions positioned from the N- to the C-terminus as V_{H}-V_{L}, while the L construct included the first binding specificity for U5 snRNP200 having the variable regions positioned from the N- to the C-terminus as V_{L}-V_{H}. See FIG. 27C.

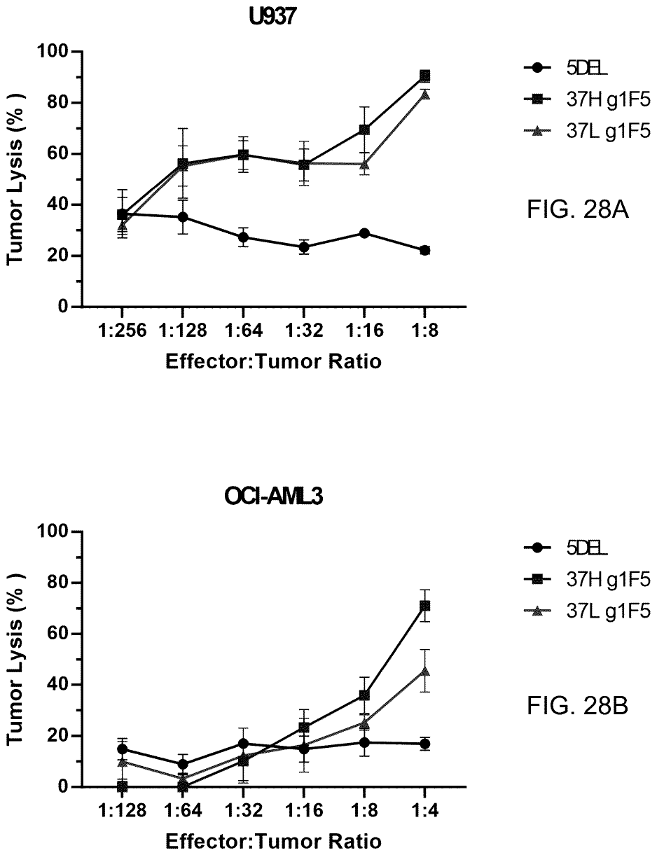

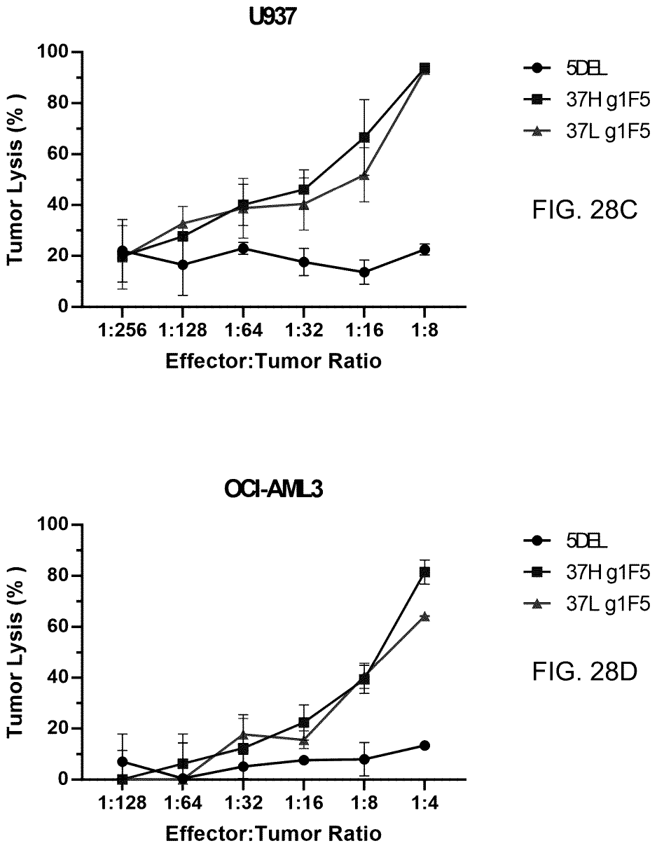

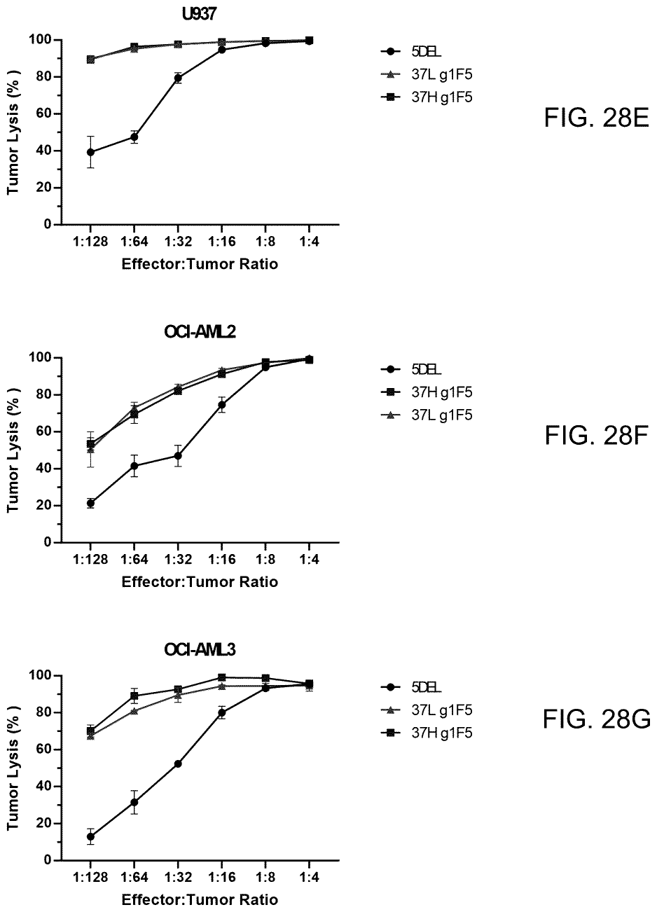

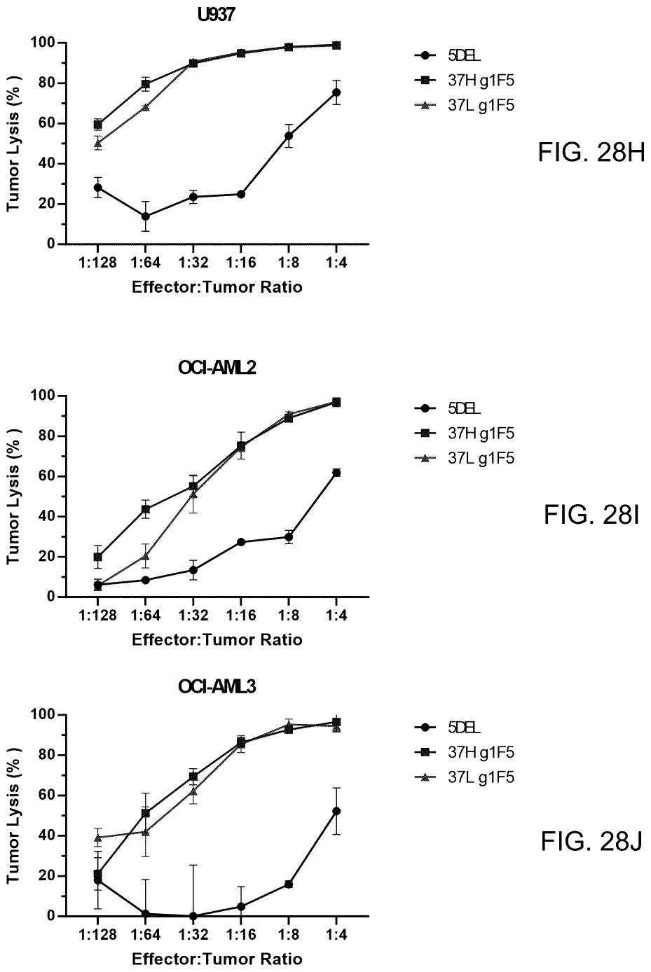

Next, T cells from multiple donors were transduced with either the 37Hg1F5 having the H construct for the U5 snRNP200 binding specificity and the 01F05C01 scFv in a V_{H}-V_{L} orientation or the 37Lg1F5 construct having the L construct for the U5 snRNP200 binding specificity and the 01F05C01 scFv in a V_{H}-V_{L} orientation. Further, T cells designated as 5DEL were transduced with a non-functional CAR containing a CD28 transmembrane domain and a truncated CD28 intracellular signaling domain, thus preventing the generation of intracellular signaling. The engineered T cells were co-cultured with AML cells expressing GFP-luciferase (U937 gL, OCI-AML2 gL, and/or OCI-AML3 gL; 10,000 cells/well) at different effector: tumor ratios and tumor cell lysis was measured. As seen in FIGS. 28A-28J, T cells secreting the bispecific antibody (e.g., 37Hg1F5, 37Lg1F5) exhibited superior anti-tumor effect compared to control T cells (e.g., 5DEL T cells). Thus, the presently disclosed multispecific antibodies exhibited superior anti-tumor effects.

### Example 5

To further study the effects of the presently disclosed multispecific antibodies, T cells were engineered to express 1) a CAR specific for CD371 including a CD28 co-stimulatory domain and a CD3zeta activating domain (designated as mCh_B10h28Z); 2) a bispecific antibody including a first binding specificity for CD371 and a second binding specificity for CD3 (designated as mCh_B10g1F5); or a CAR specific for CD371 including a CD28 co-stimulatory domain and a CD3zeta activating domain (designated as mCh_B10h28Z); 3) a CAR specific for CD371 including a CD28 co-stimulatory domain and a CD3zeta activating domain and a bispecific antibody including a first binding specificity for CD371 and a second binding specificity for CD3 (designated as mCh_B10h28ZpB10g1F5). The bispecific antibody included includes heavy chain variable region and light chain variable region of the anti-CD371 antibody designated as B10 described in International Patent Publication No. WO2021050857 which is incorporated by reference in its entirety, while the second binding specificity includes the heavy chain variable region and light chain variable region of the 01F05C01 scFv described in Section 3.1.

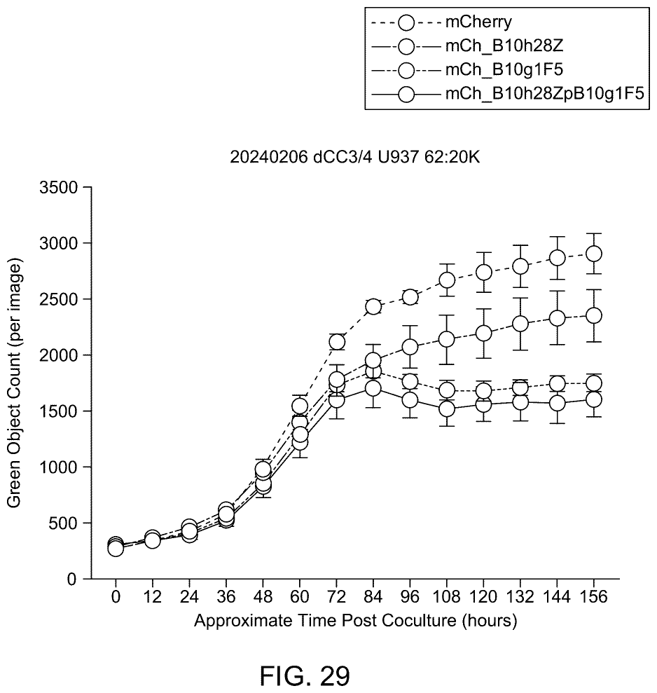

The T cells were cocultured with U937gL (AML cell line expressing GFP-luciferase) for approximately 156 hours at an effector: tumor ratio of approximately 62 T cells to 20,000 tumor cells. The GFP signal was measured every 12 hours using the IncuCyte S3, and this signal was plotted on the Y-axis of FIG. 29. T cells secreting bispecific antibodies (either alone or in combination with co-expressing a CAR) outperformed the standard 2nd-generation CAR T cell mCh_B10h28Z. Further statistical details can be found in the table below:

| Tukey's multiple | Mean | 95.00% CI | Below | | Adjusted |
|---|---|---|---|---|---|
| comparisons test | Diff. | of diff. | threshold? | Summary | P Value |
| At 156 hours | | | | | |
| mCherry vs. | 551.4 | 99.64 to | Yes | ** | 0.0098 |
| mCh_B10h28Z | | 1003 | | | |
| mCherry vs. | 1153 | 701.4 to | Yes | **** | <0.0001 |
| mCh_B10g1F5 | | 1605 | | | |
| mCherry vs. | 1298 | 846.1 to | Yes | **** | <0.0001 |
| mCh_B10h28ZpB10g1F5 | | 1750 | | | |
| mCh_B10h28Z vs. | 601.8 | 150.0 to | Yes | ** | 0.0038 |
| mCh_B10g1F5 | | 1054 | | | |
| mCh_B10h28Z vs. | 746.5 | 294.7 to | Yes | *** | 0.0002 |
| mCh_B10h28ZpB10g1F5 | | 1198 | | | |
| mCh_B10g1F5 vs. | 144.8 | −307.0 to | No | ns | 0.8395 |
| mCh_B10h28ZpB10g1F5 | | 596.5 | | | |

Accordingly, the presently disclosed multispecific antibodies exhibited superior anti-tumor effects.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An anti-CD3 antibody or an antigen-binding fragment thereof, comprising:
(a) a heavy chain variable region comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising CDR1 comprising the amino acid sequence set forth in SEQ ID NO: 9, a CDR2 comprising an amino acid sequence set forth in SEQ ID NO: 10, a CDR3 comprising the amino acid sequence set forth in SEQ ID NO: 11; and a light chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14;
(c) a heavy chain variable region comprising CDR1 comprising the amino acid sequence set forth in SEQ ID NO: 17, a CDR2 comprising an amino acid sequence set forth in SEQ ID NO: 18, a CDR3 comprising the amino acid sequence set forth in SEQ ID NO: 19; and a light chain variable region comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21;
(d) a heavy chain variable region comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a light chain variable region comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29;
(e) a heavy chain variable region comprising CDR1 comprising the amino acid sequence set forth in SEQ ID NO: 32, a CDR2 comprising an amino acid sequence set forth in SEQ ID NO: 33, a CDR3 comprising the amino acid sequence set forth in SEQ ID NO: 34; and a light chain variable region comprising a CDR1 comprising the amino acid sequence set forth in SEQ ID NO: 35, a CDR2 comprising the amino acid sequence set forth in SEQ ID NO: 36, and a CDR3 comprising the amino acid sequence set forth in SEQ ID NO: 37;
(f) a heavy chain variable region comprising a CDR1 comprising the amino acid sequence set forth in SEQ ID NO: 40, a CDR2 comprising the amino acid sequence set forth in SEQ ID NO: 18, a CDR3 comprising the amino acid sequence set forth in SEQ ID NO: 41; and a light chain variable region comprising a CDR1 comprising the amino acid sequence set forth in SEQ ID NO: 42, a CDR2 comprising the amino acid sequence set forth in SEQ ID NO: 13, and a CDR3 comprising the amino acid sequence set forth in SEQ ID NO: 21;
(g) a heavy chain variable region comprising CDR1 comprising the amino acid sequence set forth in SEQ ID NO: 45, a CDR2 comprising an amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21;
(h) a heavy chain variable region comprising CDR1 comprising the amino acid sequence set forth in SEQ ID NO: 24, a CDR2 comprising an amino acid sequence set forth in SEQ ID NO: 49, a CDR3 comprising the amino acid sequence set forth in SEQ ID NO: 26; and a light chain variable region comprising a CDR1 comprising the amino acid sequence set forth in SEQ ID NO: 20, a CDR2 comprising the amino acid sequence set forth in SEQ ID NO: 28, and a CDR3 comprising the amino acid sequence set forth in SEQ ID NO: 50;
(i) a heavy chain variable region comprising a CDR1 comprising the amino acid sequence set forth in SEQ ID NO: 9, a CDR2 comprising the amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 53, a CDR2 comprising the amino acid sequence set forth in SEQ ID NO: 13, and a CDR3 comprising the amino acid sequence set forth in SEQ ID NO: 21;
(j) a heavy chain variable region comprising CDR1 comprising the amino acid sequence set forth in SEQ ID NO: 9, a CDR2 comprising an amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 21;
(k) a heavy chain variable region comprising CDR1 comprising the amino acid sequence set forth in SEQ ID NO: 9, a CDR2 comprising an amino acid sequence set forth in SEQ ID NO: 18, a CDR3 comprising the amino acid sequence set forth in SEQ ID NO: 46; and a light chain variable region comprising a CDR1 comprising the amino acid sequence set forth in SEQ ID NO: 58, a CDR2 comprising the amino acid sequence set forth in SEQ ID NO: 13, and a CDR3 comprising the amino acid sequence set forth in SEQ ID NO: 21; or
(l) a heavy chain variable region comprising a CDR1 comprising the amino acid sequence set forth in SEQ ID NO: 61, a CDR2 comprising the amino acid sequence set forth in SEQ ID NO: 62, a CDR3 comprising the amino acid sequence set forth in SEQ ID NO: 63; and a light chain variable region comprising a CDR1 comprising the amino acid sequence set forth in SEQ ID NO: 4, a CDR2 comprising the amino acid sequence set forth in SEQ ID NO: 64, and a CDR3 comprising the amino acid sequence set forth in SEQ ID NO: 65.

2. The anti-CD3 antibody or antigen-binding fragment thereof of claim 1, comprising:
(a) a heavy chain variable region comprising a CDR1 comprising the amino acid sequence set forth in SEQ ID NO: 1, a CDR2 comprising the amino acid sequence set forth in SEQ ID NO: 2, a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a heavy chain variable region comprising CDR1 comprising the amino acid sequence set forth in SEQ ID NO: 9, a CDR2 comprising an amino acid sequence set forth in SEQ ID NO: 10, a CDR3 comprising the amino acid sequence set forth in SEQ ID NO: 11; and a light chain variable region comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14;
(c) a heavy chain variable region comprising CDR1 comprising the amino acid sequence set forth in SEQ ID NO: 17, a CDR2 comprising an amino acid sequence set forth in SEQ ID NO: 18, a CDR3 comprising the amino acid sequence set forth in SEQ ID NO: 19; and a light chain variable region comprising a CDR1 comprising the amino acid sequence set forth in SEQ ID NO: 20, a CDR2 comprising the amino acid sequence set forth in SEQ ID NO: 13, and a CDR3 comprising the amino acid sequence set forth in SEQ ID NO: 21; or
(d) a heavy chain variable region comprising a CDR1 comprising the amino acid sequence set forth in SEQ ID NO: 24, a CDR2 comprising the amino acid sequence set forth in SEQ ID NO: 25, a CDR3 comprising the amino acid sequence set forth in SEQ ID NO: 26; and a light chain variable region comprising a CDR1 comprising the amino acid sequence set forth in SEQ ID NO: 27, a CDR2 comprising the amino acid sequence set forth in SEQ ID NO: 28, and a CDR3 comprising the amino acid sequence set forth in SEQ ID NO: 29.

3. The anti-CD3 antibody or antigen-binding fragment thereof of claim 1, comprising a heavy chain variable region and a light chain variable region, wherein the heavy chain variable region and the light chain variable region are selected from the group consisting of:
(a) a heavy chain variable region comprising an amino acid sequence that is at least about 80% identical to the amino acid sequence set forth in SEQ ID NO: 7, and a light chain variable region comprising an amino acid sequence that is at least about 80% identical to the amino acid sequence set forth in SEQ ID NO: 8;
(b) a heavy chain variable region comprising an amino acid sequence that is at least about 80% identical to the amino acid sequence set forth in SEQ ID NO: 15, and a light chain variable region comprising an amino acid sequence that is at least about 80% identical to the amino acid sequence set forth in SEQ ID NO: 16;
(c) a heavy chain variable region comprising an amino acid sequence that is at least about 80% identical to the amino acid sequence set forth in SEQ ID NO: 22, and a light chain variable region comprising an amino acid sequence that is at least about 80% identical to the amino acid sequence set forth in SEQ ID NO: 23;
(d) a heavy chain variable region comprising an amino acid sequence that is at least about 80% identical to the amino acid sequence set forth in SEQ ID NO: 30, and a light chain variable region comprising an amino acid sequence that is at least about 80% identical to the amino acid sequence set forth in SEQ ID NO: 31;
(e) a heavy chain variable region comprising an amino acid sequence that is at least about 80% identical to the amino acid sequence set forth in SEQ ID NO: 38, and a light chain variable region comprising an amino acid sequence that is at least about 80% identical to the amino acid sequence set forth in SEQ ID NO: 39;
(f) a heavy chain variable region comprising an amino acid sequence that is at least about 80% identical to the amino acid sequence set forth in SEQ ID NO: 43, and a light chain variable region comprising an amino acid sequence that is at least about 80% identical to the amino acid sequence set forth in SEQ ID NO: 44;
(g) a heavy chain variable region comprising an amino acid sequence that is at least about 80% identical to the amino acid sequence set forth in SEQ ID NO: 47, and a light chain variable region comprising an amino acid sequence that is at least about 80% identical to the amino acid sequence set forth in SEQ ID NO: 48;
(h) a heavy chain variable region comprising an amino acid sequence that is at least about 80% identical to the amino acid sequence set forth in SEQ ID NO: 51, and a light chain variable region comprising an amino acid sequence that is at least about 80% identical to the amino acid sequence set forth in SEQ ID NO: 52;
(i) a heavy chain variable region comprising an amino acid sequence that is at least about 80% identical to the amino acid sequence set forth in SEQ ID NO: 54, and a light chain variable region comprising an amino acid sequence that is at least about 80% identical to the amino acid sequence set forth in SEQ ID NO: 55;
(j) a heavy chain variable region comprising an amino acid sequence that is at least about 80% identical to the amino acid sequence set forth in SEQ ID NO: 56, and a light chain variable region comprising an amino acid sequence that is at least about 80% identical to the amino acid sequence set forth in SEQ ID NO: 57;
(k) a heavy chain variable region comprising an amino acid sequence that is at least about 80% identical to the amino acid sequence set forth in SEQ ID NO: 59, and a light chain variable region comprising an amino acid sequence that is at least about 80% identical to the amino acid sequence set forth in SEQ ID NO: 60; and
(l) a heavy chain variable region comprising an amino acid sequence that is at least about 80% identical to the amino acid sequence set forth in SEQ ID NO: 66, and a light chain variable region comprising an amino acid sequence that is at least about 80% identical to the amino acid sequence set forth in SEQ ID NO: 67.

4. The anti-CD3 antibody or antigen-binding fragment thereof of claim 1, wherein:
(a) the heavy chain variable region comprises the amino acid sequence set forth in SEQ ID NO: 7, and the light chain variable region comprises the amino acid sequence set forth in SEQ ID NO: 8;
(b) the heavy chain variable region comprises the amino acid sequence set forth in SEQ ID NO: 15, and the light chain variable region comprises the amino acid sequence set forth in SEQ ID NO: 16;
(c) the heavy chain variable region comprises the amino acid sequence set forth in SEQ ID NO: 22, and the light chain variable region comprises the amino acid sequence set forth in SEQ ID NO: 23;
(d) the heavy chain variable region comprises the amino acid sequence set forth in SEQ ID NO: 30, and the light chain variable region comprises the amino acid sequence set forth in SEQ ID NO: 31;
(e) the heavy chain variable region comprises the amino acid sequence set forth in SEQ ID NO: 38, and the light chain variable region comprises the amino acid sequence set forth in SEQ ID NO: 39;
(f) the heavy chain variable region comprises the amino acid sequence set forth in SEQ ID NO: 43, and the light chain variable region comprises the amino acid sequence set forth in SEQ ID NO: 44;
(g) the heavy chain variable region comprises the amino acid sequence set forth in SEQ ID NO: 47, and the light chain variable region comprises the amino acid sequence set forth in SEQ ID NO: 48;
(h) the heavy chain variable region comprises the amino acid sequence set forth in SEQ ID NO: 51, and the light chain variable region comprises the amino acid sequence set forth in SEQ ID NO: 52;
(i) the heavy chain variable region comprises the amino acid sequence set forth in SEQ ID NO: 54, and the light chain variable region comprises the amino acid sequence set forth in SEQ ID NO: 55;
(j) the heavy chain variable region comprises the amino acid sequence set forth in SEQ ID NO: 56, and the light chain variable region comprises the amino acid sequence set forth in SEQ ID NO: 57;
(k) the heavy chain variable region comprises the amino acid sequence set forth in SEQ ID NO: 59, and the light chain variable region comprises the amino acid sequence set forth in SEQ ID NO: 60; or
(l) the heavy chain variable region comprises the amino acid sequence set forth in SEQ ID NO: 66, and the light chain variable region comprises the amino acid sequence set forth in SEQ ID NO: 67.
The anti-CD3 antibody or antigen-binding fragment thereof of claim 1, wherein the antibody comprises a heavy chain constant region and/or a light chain constant region, wherein:
(a) the heavy chain constant region comprises an amino acid sequence that is about 80% identical to the amino acid sequence set forth in SEQ ID NO: 82, SEQ ID NO: 83, SEQ ID NO: 84, or SEQ ID NO: 85; and/or
(b) the light chain constant region comprises an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 86.

5. The anti-CD3 antibody or antigen-binding fragment thereof of claim 1, wherein the antigen-binding fragment is a Fab, Fab′, F(ab′)2, variable fragment (Fv), or single chain variable region (scFv).

6. A composition comprising the anti-CD3 antibody or antigen-binding fragment thereof of claim 1.

7. An immunoconjugate comprising the anti-CD3 antibody or antigen-binding fragment thereof of claim 1, linked to a therapeutic agent.

8. A multi-specific molecule comprising a first binding specificity for CD3 and a second binding specificity, wherein the first binding specificity comprises the antibody or antigen-binding fragment thereof of claim 1.

9. The multi-specific molecule of claim 8, wherein:
(a) the second binding specificity comprises an anti-CD33 antibody or antigen-binding fragment thereof comprising a V_{H} comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a V_{L} comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92;
(b) the second binding specificity comprises an anti-CD33 antibody or antigen-binding fragment thereof comprising a V_{H} comprising the amino acid sequence set forth in SEQ ID NO: 93, and a V_{L} comprising the amino acid sequence set forth in SEQ ID NO: 94;
(c) the second binding specificity comprises an anti-U5 snRNP200 antibody or antigen-binding fragment thereof comprising a V_{H} comprising a CDR1 comprising the amino acid sequence set forth in SEQ ID NO: 116, a CDR2 comprising the amino acid sequence set forth in SEQ ID NO: 117, and a CDR3 comprising the amino acid sequence set forth in SEQ ID NO: 118; and a V_{L} comprising a CDR1 comprising the amino acid sequence set forth in SEQ ID NO: 119, a CDR2 comprising the amino acid sequence set forth in SEQ ID NO: 120, and a CDR3 comprising the amino acid sequence set forth in SEQ ID NO: 121;
(d) the second binding specificity comprises an anti-U5 snRNP200 antibody or antigen-binding fragment thereof comprising a V_{H} comprising the amino acid sequence set forth in SEQ ID NO: 122, and a V_{L} comprising the amino acid sequence set forth in SEQ ID NO: 123;
(e) the second binding specificity comprises an anti-CD371 antibody or antigen-binding fragment thereof comprising a V_{H} comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134; and a V_{L} comprising a CDR1 comprising the amino acid sequence set forth in SEQ ID NO: 135, a CDR2 comprising the amino acid sequence set forth in SEQ ID NO: 136, and a CDR3 comprising the amino acid sequence set forth in SEQ ID NO: 137; or
(f) the second binding specificity comprises an anti-CD371 antibody or antigen-binding fragment thereof comprising a V_{H} comprising the amino acid sequence set forth in SEQ ID NO: 138, and a V_{L} comprising the amino acid sequence set forth in SEQ ID NO: 139.

10. A composition comprising the multi-specific molecule of claim 8.

11. An immunocytokine comprising an antibody or antigen-binding fragment thereof of claim 1 and an IL-18 polypeptide or a fragment thereof.

12. An immunocytokine comprising a multispecific molecule of claim 8 and an IL-18 polypeptide or a fragment thereof.

13. A nucleic acid that encodes an antibody or antigen-binding fragment thereof of claim 1.

14. A vector comprising the nucleic acid of claim 13.

15. A host cell comprising the vector of claim 14.

16. An immunoresponsive cell comprising the antibody or antigen-binding fragment thereof of claim 1.

17. A method of treating or ameliorating a disease or disorder in a subject, reducing tumor burden in a subject, treating and/or preventing a tumor in a subject, and/or increasing or lengthening survival of a subject having a tumor, comprising administering to the subject the antibody or antigen-binding fragment thereof of claim 1.

18. The method of claim 17, wherein the tumor is hematological cancer or solid tissue cancer.

19. The method of claim 18, wherein the hematological cancer is selected from the group consisting of acute myeloid leukemia (AML), myelodysplastic syndromes (MDS), acute lymphoblastic leukemia (ALL), chronic lymphocytic leukemia (CLL), myeloproliferative neoplasms (MPNs), and chronic myeloid neoplasms.

20. A kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof of claim 1.
